SEQUENCE LISTING (18 sequences)

SEQ ID NO: 1 - protein, 2701 aa, Homo sapiens
MAEEGAVAVCVRVRPLNSREESLGETAQVYWKTDNNVIYQVDGSKSFNFDRVFHGNETTKNVYEEIAAPIIDSAIQGYNGTIFAYGQTASGKTYTMMGSEDHLGVIPRAIHDIFQKIKKFPDREFLLRVSYMEIYNETITDLLCGTQKMKPLIIREDVNRNVYVADLTEEVVYTSEMALKWITKGEKSRHYGETKMNQRSSRSHTIFRMILESREKGEPSNCEGSVKVSHLNLVDLAGSERAAQTGAAGVRLKEGCNINRSLFILGQVIKKLSDGQVGGFINYRDSKLTRILQNSLGGNAKTRIICTITPVSFDETLTALQFASTAKYMKNTPYVNEVSTDEALLKRYRKEIMDLKKQLEEVSLETRAQAMEKDQLAQLLEEKDLLQKVQNEKIENLTRMLVTSSSLTLQQELKAKRKRRVTWCLGKINKMKNSNYADQFNIPTNITTKTHKLSINLLREIDESVCSESDVFSNTLDTLSEIEWNPATKLLNQENIESELNSLRADYDNLVLDYEQLRTEKEEMELKLKEKNDLDEFEALERKTKKDQEMQLIHEISNLKNLVKHAEVYNQDLENELSSKVELLREKEDQIKKLQEYIDSQKLENIKMDLSYSLESIEDPKQMKQTLFDAETVALDAKRESAFLRSENLELKEKMKELATTYKQMENDIQLYQSQLEAKKKMQVDLEKELQSAFNEITKLTSLIDGKVPKDLLCNLELEGKITDLQKELNKEVEENEALREEVILLSELKSLPSEVERLRKEIQDKSEELHIITSEKDKLFSEVVHKESRVQGLLEEIGKTKDDLATTQSNYKSTDQEFQNFKTLHMDFEQKYKMVLEENERMNQEIVNLSKEAQKFDSSLGALKTELSYKTQELQEKTREVQERLNEMEQLKEQLENRDSTLQTVEREKTLITEKLQQTLEEVKTLTQEKDDLKQLQESLQIERDQLKSDIHDTVNMNIDTQEQLRNALESLKQHQETINTLKSKISEEVSRNLHMEENTGETKDEFQQKMVGIDKKQDLEAKNTQTLTADVKDNEIIEQQRKIFSLIQEKNELQQMLESVIAEKEQLKTDLKENIEMTIENQEELRLLGDELKKQQEIVAQEKNHAIKKEGELSRTCDRLAEVEEKLKEKSQQLQEKQQQLLNVQEEMSEMQKKINEIENLKNELKNKELTLEHMETERLELAQKLNENYEEVKSITKERKVLKELQKSFETERDHLRGYIREIEATGLQTKEELKIAHIHLKEHQETIDELRRSVSEKTAQIINTQDLEKSHTKLQEEIPVLHEEQELLPNVKEVSETQETMNELELLTEQSTTKDSTTLARIEMERLRLNEKFQESQEEIKSLTKERDNLKTIKEALEVKHDQLKEHIRETLAKIQESQSKQEQSLNMKEKDNETTKIVSEMEQFKPKDSALLRIEIEMLGLSKRLQESHDEMKSVAKEKDDLQRLQEVLQSESDQLKENIKEIVAKHLETEEELKVAHCCLKEQEETINELRVNLSEKETEISTIQKQLEAINDKLQNKIQEIYEKEEQFNIKQISEVQEKVNELKQFKEHRKAKDSALQSIESKMLELTNRLQESQEEIQIMIKEKEEMKRVQEALQIERDQLKENTKEIVAKMKESQEKEYQFLKMTAVNETQEKMCEIEHLKEQFETQKLNLENIETENIRLTQILHENLEEMRSVTKERDDLRSVEETLKVERDQLKENLRETITRDLEKQEELKIVHMHLKEHQETIDKLRGIVSEKTNEISNMQKDLEHSNDALKAQDLKIQEELRIAHMHLKEQQETIDKLRGIVSEKTDKLSNMQKDLENSNAKLQEKIQELKANEHQLITLKKDVNETQKKVSEMEQLKKQIKDQSLTLSKLEIENLNLAQKLHENLEEMKSVMKERDNLRRVEETLKLERDQLKESLQETKARDLEIQQELKTARMLSKEHKETVDKLREKISEKTIQISDIQKDLDKSKDELQKKIQELQKKELQLLRVKEDVNMSHKKINEMEQLKKQFEAQNLSMQSVRMDNFQLTKKLHESLEEIRIVAKERDELRRIKESLKMERDQFIATLREMIARDRQNHQVKPEKRLLSDGQQHLTESLREKCSRIKELLKRYSEMDDHYECLNRLSLDLEKEIEFQKELSMRVKANLSLPYLQTKHIEKLFTANQRCSMEFHRIMKKLKYVLSYVTKIKEEQHESINKFEMDFIDEVEKQKELLIKIQHLQQDCDVPSRELRDLKLNQNMDLHIEEILKDFSESEFPSIKTEFQQVLSNRKEMTQFLEEWLNTRFDIEKLKNGIQKENDRICQVNNFFNNRIIAIMNESTEFEERSATISKEWEQDLKSLKEKNEKLFKNYQTLKTSLASGAQVNPTTQDNKNPHVTSRATQLTTEKIRELENSLHEAKESAMHKESKIIKMQKELEVTNDIIAKLQAKVHESNKCLEKTKETIQVLQDKVALGAKPYKEEIEDLKMKLVKIDLEKMKNAKEFEKEISATKATVEYQKEVIRLLRENLRRSQQAQDTSVISEHTDPQPSNKPLTCGGGSGIVQNTKALILKSEHIRLEKEISKLKQQNEQLIKQKNELLSNNQHLSNEVKTWKERTLKREAHKQVTCENSPKSPKVTGTASKKKQITPSQCKERNLQDPVPKESPKSCFFDSRSKSLPSPHPVRYFDNSSLGLCPEVQNAGAESVDSQPGPWHASSGKDVPECKTQ

SEQ ID NO: 2 - DNA, 8630 bp, Homo sapiens
  source 1..8630: mol_type = genomic DNA
taaatttaaaggcggggcggcctgtgagccctgaagtgccggccgcggagggtcctggccattttcctgggaccagttcagcctgataggatggcggaggaaggagccgtggccgtctgcgtgcgagtgcggccgctgaacagcagagaagaatcacttggagaaactgcccaagtttactggaaaactgacaataatgtcatttatcaagttgatggaagtaaatccttcaattttgatcgtgtctttcatggtaatgaaactaccaaaaatgtgtatgaagaaatagcagcaccaatcatcgattctgccatacaaggctacaatggtactatatttgcctatggacagactgcttcaggaaaaacatataccatgatgggttcagaagatcatttgggagttatacccagggcaattcatgacattttccaaaaaattaagaagtttcctgatagggaatttctcttacgtgtatcttacatggaaatatacaatgaaaccattacagatttactctgtggcactcaaaaaatgaaacctttaattattcgagaagatgtcaataggaatgtgtatgttgctgatctcacagaagaagttgtatatacatcagaaatggctttgaaatggattacaaagggagaaaagagcaggcattatggagaaacaaaaatgaatcaaagaagcagtcgttctcataccatctttaggatgattttggaaagcagagagaagggtgaaccttctaattgtgaaggatctgttaaggtatcccatttgaatttggttgatcttgcaggcagtgaaagagctgctcaaacaggcgctgcaggtgtgcggctcaaggaaggctgtaatataaatcgaagcttatttattttgggacaagtgatcaagaaacttagtgatggacaagttggtggtttcataaattatcgagatagcaagttaacacgaattctccagaattccttgggaggaaatgcaaagacacgtattatctgcacaattactccagtatcttttgatgaaacacttactgctctccagtttgccagtactgctaaatatatgaagaatactccttatgttaatgaggtatcaactgatgaagctctcctgaaaaggtatagaaaagaaataatggatcttaaaaaacaattagaggaggtttctttagagacgcgggctcaggcaatggaaaaagaccaattggcccaacttttggaagaaaaagatttgcttcagaaagtacagaatgagaaaattgaaaacttaacacggatgctggtgacctcttcttccctcacgttgcaacaggaattaaaggctaaaagaaaacgaagagttacttggtgccttggcaaaattaacaaaatgaagaactcaaactatgcagatcaatttaatataccaacaaatataacaacaaaaacacataagctttctataaatttattacgagaaattgatgaatctgtctgttcagagtctgatgttttcagtaacactcttgatacattaagtgagatagaatggaatccagcaacaaagctactaaatcaggagaatatagaaagtgagttgaactcacttcgtgctgactatgataatctggtattagactatgaacaactacgaacagaaaaagaagaaatggaattgaaattaaaagaaaagaatgatttggatgaatttgaggctctagaaagaaaaactaaaaaagatcaagagatgcaactaattcatgaaatttcgaacttaaagaatttagttaagcatgcagaagtatataatcaagatcttgagaatgaactcagttcaaaagtagagctgcttagagaaaaggaagaccagattaagaagctacaggaatacatagactctcaaaagctagaaaatataaaaatggacttgtcatactcattggaaagcattgaagacccaaaacaaatgaagcagactctgtttgatgctgaaactgtagcccttgatgccaagagagaatcagcctttcttagaagtgaaaatctggagctgaaggagaaaatgaaagaacttgcaactacatacaagcaaatggaaaatgatattcagttatatcaaagccagttggaggcaaaaaagaaaatgcaagttgatctggagaaagaattacaatctgcttttaatgagataacaaaactcacctcccttatagatggcaaagttccaaaagatttgctctgtaatttggaattggaaggaaagattactgatcttcagaaagaactaaataaagaagttgaagaaaatgaagctttgcgggaagaagtcattttgctttcagaattgaaatctttaccttctgaagtagaaaggctgaggaaagagatacaagacaaatctgaagagctccatataataacatcagaaaaagataaattgttttctgaagtagttcataaggagagtagagttcaaggtttacttgaagaaattgggaaaacaaaagatgacctagcaactacacagtcgaattataaaagcactgatcaagaattccaaaatttcaaaacccttcatatggactttgagcaaaagtataagatggtccttgaggagaatgagagaatgaatcaggaaatagttaatctctctaaagaagcccaaaaatttgattcgagtttgggtgctttgaagaccgagctttcttacaagacccaagaacttcaggagaaaacacgtgaggttcaagaaagactaaatgagatggaacagctgaaggaacaattagaaaatagagattctacgctgcaaactgtagaaagggagaaaacactgattactgagaaactgcagcaaactttagaagaagtaaaaactttaactcaagaaaaagatgatctaaaacaactccaagaaagcttgcaaattgagagggaccaactcaaaagtgatattcacgatactgttaacatgaatatagatactcaagaacaattacgaaatgctcttgagtctctgaaacaacatcaagaaacaattaatacactaaaatcgaaaatttctgaggaagtttccaggaatttgcatatggaggaaaatacaggagaaactaaagatgaatttcagcaaaagatggttggcatagataaaaaacaggatttggaagctaaaaatacccaaacactaactgcagatgttaaggataatgagataattgagcaacaaaggaagatattttctttaatacaggagaaaaatgaactccaacaaatgttagagagtgttatagcagaaaaggaacaattgaagactgacctaaaggaaaatattgaaatgaccattgaaaaccaggaagaattaagacttcttggggatgaacttaaaaagcaacaagagatagttgcacaagaaaagaaccatgccataaagaaagaaggagagctttctaggacctgtgacagactggcagaagttgaagaaaaactaaaggaaaagagccagcaactccaagaaaaacagcaacaacttcttaatgtacaagaagagatgagtgagatgcagaaaaagattaatgaaatagagaatttaaagaatgaattaaagaacaaagaattgacattggaacatatggaaacagagaggcttgagttggctcagaaacttaatgaaaattatgaggaagtgaaatctataaccaaagaaagaaaagttctaaaggaattacagaagtcatttgaaacagagagagaccaccttagaggatatataagagaaattgaagctacaggcctacaaaccaaagaagaactaaaaattgctcatattcacctaaaagaacaccaagaaactattgatgaactaagaagaagcgtatctgagaagacagctcaaataataaatactcaggacttagaaaaatcccataccaaattacaagaagagatcccagtgcttcatgaggaacaagagttactgcctaatgtgaaagaagtcagtgagactcaggaaacaatgaatgaactggagttattaacagaacagtccacaaccaaggactcaacaacactggcaagaatagaaatggaaaggctcaggttgaatgaaaaatttcaagaaagtcaggaagagataaaatctctaaccaaggaaagagacaaccttaaaacgataaaagaagcccttgaagttaaacatgaccagctgaaagaacatattagagaaactttggctaaaatccaggagtctcaaagcaaacaagaacagtccttaaatatgaaagaaaaagacaatgaaactaccaaaatcgtgagtgagatggagcaattcaaacccaaagattcagcactactaaggatagaaatagaaatgctcggattgtccaaaagacttcaagaaagtcatgatgaaatgaaatctgtagctaaggagaaagatgacctacagaggctgcaagaagttcttcaatctgaaagtgaccagctcaaagaaaacataaaagaaattgtagctaaacacctggaaactgaagaggaacttaaagttgctcattgttgcctgaaagaacaagaggaaactattaatgagttaagagtgaatctttcagagaaggaaactgaaatatcaaccattcaaaagcagttagaagcaatcaatgataaattacagaacaagatccaagagatttatgagaaagaggaacaatttaatataaaacaaattagtgaggttcaggaaaaagtgaatgaactgaaacaattcaaggagcatcgcaaagccaaggattcagcactacaaagtatagaaagtaagatgctcgagttgaccaacagacttcaagaaagtcaagaagaaatacaaattatgattaaggaaaaagaggaaatgaaaagagtacaggaggcccttcagatagagagagaccaactgaaagaaaacactaaagaaattgtagctaaaatgaaagaatctcaagaaaaagaatatcagtttcttaagatgacagctgtcaatgagactcaggagaaaatgtgtgaaatagaacacttgaaggagcaatttgagacccagaagttaaacctggaaaacatagaaacggagaatataaggttgactcagatactacatgaaaaccttgaagaaatgagatctgtaacaaaagaaagagatgaccttaggagtgtggaggagactctcaaagtagagagagaccagctcaaggaaaaccttagagaaactataactagagacctagaaaaacaagaggagctaaaaattgttcacatgcatctgaaggagcaccaagaaactattgataaactaagagggattgtttcagagaaaacaaatgaaatatcaaatatgcaaaaggacttagaacactcaaatgatgccttaaaagcacaggatctgaaaatacaagaggaactaagaattgctcacatgcatctgaaagagcagcaggaaactattgacaaactcagaggaattgtttctgagaagacagataaactatcaaatatgcaaaaagatttagaaaattcaaatgctaaattacaagaaaagattcaagaacttaaggcaaatgaacatcaacttattacgttaaaaaaagatgtcaatgagacacagaaaaaagtgtctgaaatggagcaactaaagaaacaaataaaagaccaaagcttaactctgagtaaattagaaatagagaatttaaatttggctcagaaacttcatgaaaaccttgaagaaatgaaatctgtaatgaaagaaagagataatctaagaagagtagaggagacactcaaactggagagagaccaactcaaggaaagcctgcaagaaaccaaagctagagatctggaaatacaacaggaactaaaaactgctcgtatgctatcaaaagaacacaaagaaactgttgataaacttagagaaaaaatttcagaaaagacaattcaaatttcagacattcaaaaggatttagataaatcaaaagatgaattacagaaaaagatccaagaacttcagaaaaaagaacttcaactgcttagagtgaaagaagatgtcaatatgagtcataaaaaaattaatgaaatggaacagttgaagaagcaatttgaggcccaaaacttatctatgcaaagtgtgagaatggataacttccagttgactaagaaacttcatgaaagccttgaagaaataagaattgtagctaaagaaagagatgagctaaggaggataaaagaatctctcaaaatggaaagggaccaattcatagcaaccttaagggaaatgatagctagagaccgacagaaccaccaagtaaaacctgaaaaaaggttactaagtgatggacaacagcaccttacggaaagcctgagagaaaagtgctctagaataaaagagcttttgaagagatactcagagatggatgatcattatgagtgcttgaatagattgtctcttgacttggagaaggaaattgaattccaaaaagagctttcaatgagagttaaagcaaacctctcacttccctatttacaaaccaaacacattgaaaaactttttactgcaaaccagagatgctccatggaattccacagaatcatgaagaaactgaagtatgtgttaagctatgttacaaaaataaaagaagaacaacatgaatccatcaataaatttgaaatggattttattgatgaagtggaaaagcaaaaggaattgctaattaaaatacagcaccttcaacaagattgtgatgtaccatccagagaattaagggatctcaaattgaaccagaatatggatctacatattgaggaaattctcaaagatttctcagaaagtgagttccctagcataaagactgaatttcaacaagtactaagtaataggaaagaaatgacacagtttttggaagagtggttaaatactcgttttgatatagaaaagcttaaaaatggcatccagaaagaaaatgataggatttgtcaagtgaataacttctttaataacagaataattgccataatgaatgaatcaacagagtttgaggaaagaagtgctaccatatccaaagagtgggaacaggacctgaaatcactgaaagagaaaaatgaaaaactatttaaaaactaccaaacattgaagacttccttggcatctggtgcccaggttaatcctaccacacaagacaataagaatcctcatgttacatcaagagctacacagttaaccacagagaaaattcgagagctggaaaattcactgcatgaagctaaagaaagtgctatgcataaggaaagcaagattataaagatgcagaaagaacttgaggtgactaatgacataatagcaaaacttcaagccaaagttcatgaatcaaataaatgccttgaaaaaacaaaagagacaattcaagtacttcaggacaaagttgctttaggagctaagccatataaagaagaaattgaagatctcaaaatgaagcttgtgaaaatagacctagagaaaatgaaaaatgccaaagaatttgaaaaggaaatcagtgctacaaaagccactgtagaatatcaaaaggaagttataaggctattgagagaaaatctcagaagaagtcaacaggcccaagatacctcagtgatatcagaacatactgatcctcagccttcaaataaacccttaacttgtggaggtggcagcggcattgtacaaaacacaaaagctcttattttgaaaagtgaacatataaggctagaaaaagaaatttctaagttaaagcagcaaaatgaacagctaataaaacaaaagaatgaattgttaagcaataatcagcatctttccaatgaggtcaaaacttggaaggaaagaacccttaaaagagaggctcacaaacaagtaacttgtgagaattctccaaagtctcctaaagtgactggaacagcttctaaaaagaaacaaattacaccctctcaatgcaaggaacggaatttacaagatcctgtgccaaaggaatcaccaaaatcttgtttttttgatagccgatcaaagtctttaccatcacctcatccagttcgctattttgataactcaagtttaggcctttgtccagaggtgcaaaatgcaggagcagagagtgtggattctcagccaggtccttggcacgcctcctcaggcaaggatgtgcctgagtgcaaaactcagtagactcctctttgtcacttctctggagatccagcattccttatttggaaatgactttgtttatgtgtctatccctggtaatgatgttgtagtgcagcttaatttcaattcagtctttactttgccactagagttgaaagataagggaacaggaaatgaatgcattgtggtaatttagaatggtgatagcaataccttcttcttgcatatggtaatacttttaaaagttgaattgttttatttatttgtatattttgtaaagaataaagttattgaaagaaatgtaaagttatctacatgacttagcatattccaaagcataatacatacattaatataaaacatcattttattaacaaaattgtaaatgtttttaataccttacacattcaataaatgtttagtagttctgaatcaccaaaaaaaaaaaaaaaaaa

SEQ ID NO: 3 - protein, 504 aa, Homo sapiens
MEATGTWALLLALALLLLLTLALSGTRARGHLPPGPTPLPLLGNLLQLRPGALYSGLMRLSKKYGPVFTIYLGPWRPVVVLVGQEAVREALGGQAEEFSGRGTVAMLEGTFDGHGVFFSNGERWRQLRKFTMLALRDLGMGKREGEELIQAEARCLVETFQGTEGRPFDPSLLLAQATSNVVCSLLFGLRFSYEDKEFQAVVRAAGGTLLGVSSQGGQTYEMFSWFLRPLPGPHKQLLHHVSTLAAFTVRQVQQHQGNLDASGPARDLVDAFLLKMAQEEQNPGTEFTNKNMLMTVIYLLFAGTMTVSTTVGYTLLLLMKYPHVQKWVREELNRELGAGQAPSLGDRTRLPYTDAVLHEAQRLLALVPMGIPRTLMRTTRFRGYTLPQGTEVFPLLGSILHDPNIFKHPEEFNPDRFLDADGRFRKHEAFLPFSLGKRVCLGEGLAKAELFLFFTTILQAFSLESPCPPDTLSLKPTVSGLFNIPPAFQLQVRPTDLHSTTQTR

SEQ ID NO: 4 - DNA, 2660 bp, Homo sapiens
  source 1..2660: mol_type = genomic DNA
cctaactagcccagccgcgcggagcgcctgggagaggagaaggagccgacctgccgagatggaggcgaccggcacctgggcgctgctgctggcgctggcgctgctcctgctgctgacgctggcgctgtccgggaccagggcccgaggccacctgccccccgggcccacgccgctaccactgctgggaaacctcctgcagctacggcccggggcgctgtattcagggctcatgcggctgagtaagaagtacggaccggtgttcaccatctacctgggaccctggcggcctgtggtggtcctggttgggcaggaggctgtgcgggaggccctgggaggtcaggctgaggagttcagcggccggggaaccgtagcgatgctggaagggacttttgatggccatggggttttcttctccaacggggagcggtggaggcagctgaggaagtttaccatgcttgctctgcgggacctgggcatggggaagcgagaaggcgaggagctgatccaggcggaggcccggtgtctggtggagacattccaggggacagaaggacgcccattcgatccctccctgctgctggcccaggccacctccaacgtagtctgctccctcctctttggcctccgcttctcctatgaggataaggagttccaggccgtggtccgggcagctggtggtaccctgctgggagtcagctcccaggggggtcagacctacgagatgttctcctggttcctgcggcccctgccaggcccccacaagcagctcctccaccacgtcagcaccttggctgccttcacagtccggcaggtgcagcagcaccaggggaacctggatgcttcgggccccgcacgtgaccttgtcgatgccttcctgctgaagatggcacaggaggaacaaaacccaggcacagaattcaccaacaagaacatgctgatgacagtcatttatttgctgtttgctgggacgatgacggtcagcaccacggtcggctataccctcctgctcctgatgaaataccctcatgtccaaaagtgggtacgtgaggagctgaatcgggagctgggggctggccaggcaccaagcctaggggaccgtacccgcctcccttacaccgacgcggttctgcatgaggcgcagcggctgctggcgctggtgcccatgggaataccccgcaccctcatgcggaccacccgcttccgagggtacaccctgccccagggcacggaggtcttccccctccttggctccatcctgcatgaccccaacatcttcaagcacccagaagagttcaacccagaccgtttcctggatgcagatggacggttcaggaagcatgaggcgttcctgcccttctccttagggaagcgtgtctgccttggagagggcctggcaaaagcggagctcttcctcttcttcaccaccatcctacaagccttctccctggagagcccgtgcccgccggacaccctgagcctcaagcccaccgtcagtggccttttcaacattcccccagccttccagctgcaagtccgtcccactgaccttcactccaccacgcagaccagatgaaggaaggcaacttggaagtggtgggtgcccaggacggtgcctccagcctcaacagtgggcatggacagggttaatgtctccagagtgtacactgcaggcagccacatttacacgcctgcagttgttttccggagtctgtcccacggcccacacgctcacttgactcatgctgctaagatgcacaaccgcacacccatacacaactacaagggccacaaagcaactgctgggttagctttccacagacataaatatagtccatctgcaatcacaagcacatagccaggtaacccaccaactcccctggatctgcagcccacacgtgggagtctggctgtcaccttcacaagccacagaaacggccacacatgttcacagctcacacgccctctccattcatcgaacttctcagtgtccctgtccctggtgcctggcacagggaacagcatgccccctccggggtcatgccacccagagactgtcgctgtctatggccccaactcatgctccctctcttggctacaccactctcccagcctgtgaccaccgatgtccacacacccccaaccacttgtccacacagctacccacgtacgacatcgtcctggctccccagagtatcttcccactgagacacgccgcccccacagaggcacagtccccagccacctctgcaactgcagccctcagtcacccctttttaagcaccctgattctaccaaatgcaaacacatctgggtctgcgattatgcacagagactttggacatacgaggaccctcagaccggaggaacacctgcccaaccccaacacgtgcttatgtaaccacgtggaaagcggcccctgctgcccctccacacacacatacacactcactgatctacagcccctgttcggcgtcagagtccccactagacccagtggaaggggttagagaccaagtaggggccagtttccaattcaccctgtcagggagtgagccggatctgacgttccttgtgacttaagggtccggcttgggaattaaagtttgtttctggcctttagcctaaaaaaaaaaaaaaaaaaaaaaaaaaaaaaaaaaaaa

SEQ ID NO: 5 - protein, 257 aa, Homo sapiens
MAPAMESPTLLCVALLFFAPDGVLAVPQKPKVSLNPPWNRIFKGENVTLTCNGNNFFEVSSTKWFHNGSLSEETNSSLNIVNAKFEDSGEYKCQHQQVNESEPVYLEVFSDWLLLQASAEVVMEGQPLFLRCHGWRNWDVYKVIYYKDGEALKYWYENHNISITNATVEDSGTYYCTGKVWQLDYESEPLNITVIKAPREKYWLQFFIPLLVVILFAVDTGLFISTQQQVTFLLKIKRTRKGFRLLNPHPKPNPKNN

SEQ ID NO: 6 - DNA, 1198 bp, Homo sapiens
  source 1..1198: mol_type = genomic DNA
tactaagagtctccagcatcctccacctgtctaccaccgagcatgggcctatatttgaagccttagatctctccagcacagtaagcaccaggagtccatgaagaagatggctcctgccatggaatcccctactctactgtgtgtagccttactgttcttcgctccagatggcgtgttagcagtccctcagaaacctaaggtctccttgaaccctccatggaatagaatatttaaaggagagaatgtgactcttacatgtaatgggaacaatttctttgaagtcagttccaccaaatggttccacaatggcagcctttcagaagagacaaattcaagtttgaatattgtgaatgccaaatttgaagacagtggagaatacaaatgtcagcaccaacaagttaatgagagtgaacctgtgtacctggaagtcttcagtgactggctgctccttcaggcctctgctgaggtggtgatggagggccagcccctcttcctcaggtgccatggttggaggaactgggatgtgtacaaggtgatctattataaggatggtgaagctctcaagtactggtatgagaaccacaacatctccattacaaatgccacagttgaagacagtggaacctactactgtacgggcaaagtgtggcagctggactatgagtctgagcccctcaacattactgtaataaaagctccgcgtgagaagtactggctacaattttttatcccattgttggtggtgattctgtttgctgtggacacaggattatttatctcaactcagcagcaggtcacatttctcttgaagattaagagaaccaggaaaggcttcagacttctgaacccacatcctaagccaaaccccaaaaacaactgatataattactcaagaaatatttgcaacattagtttttttccagcatcagcaattgctactcaattgtcaaacacagcttgcaatatacatagaaacgtctgtgctcaaggatttatagaaatgcttcattaaactgagtgaaactggttaagtggcatgtaatagtaagtgctcaattaacattggttgaataaatgagagaatgaatagattcatttattagcatttgtaaaagagatgttcaatttcaataaaataaatataaaaccatgtaacagaatgcttctgagtaaaaaaaaaaaaaaaaaaaaaaaaaaa

SEQ ID NO: 7 - protein, 177 aa, Homo sapiens
MEICRGLRSHLITLLLFLFHSETICRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 8 - DNA, 1794 bp, Homo sapiens
  source 1..1794: mol_type = genomic DNA
atttctttataaaccacaactctgggcccgcaatggcagtccactgccttgctgcagtcacagaatggaaatctgcagaggcctccgcagtcacctaatcactctcctcctcttcctgttccattcagagacgatctgccgaccctctgggagaaaatccagcaagatgcaagccttcagaatctgggatgttaaccagaagaccttctatctgaggaacaaccaactagttgctggatacttgcaaggaccaaatgtcaatttagaagaaaagatagatgtggtacccattgagcctcatgctctgttcttgggaatccatggagggaagatgtgcctgtcctgtgtcaagtctggtgatgagaccagactccagctggaggcagttaacatcactgacctgagcgagaacagaaagcaggacaagcgcttcgccttcatccgctcagacagtggccccaccaccagttttgagtctgccgcctgccccggttggttcctctgcacagcgatggaagctgaccagcccgtcagcctcaccaatatgcctgacgaaggcgtcatggtcaccaaattctacttccaggaggacgagtagtactgcccaggcctgcctgttcccattcttgcatggcaaggactgcagggactgccagtccccctgccccagggctcccggctatgggggcactgaggaccagccattgaggggtggaccctcagaaggcgtcacaacaacctggtcacaggactctgcctcctcttcaactgaccagcctccatgctgcctccagaatggtctttctaatgtgtgaatcagagcacagcagcccctgcacaaagcccttccatgtcgcctctgcattcaggatcaaaccccgaccacctgcccaacctgctctcctcttgccactgcctcttcctccctcattccaccttcccatgccctggatccatcaggccacttgatgacccccaaccaagtggctcccacaccctgttttacaaaaaagaaaagaccagtccatgagggaggtttttaagggtttgtggaaaatgaaaattaggatttcatgatttttttttttcagtccccgtgaaggagagcccttcatttggagattatgttctttcggggagaggctgaggacttaaaatattcctgcatttgtgaaatgatggtgaaagtaagtggtagcttttcccttctttttcttctttttttgtgatgtcccaacttgtaaaaattaaaagttatggtactatgttagccccataattttttttttccttttaaaacacttccataatctggactcctctgtccaggcactgctgcccagcctccaagctccatctccactccagattttttacagctgcctgcagtactttacctcctatcagaagtttctcagctcccaaggctctgagcaaatgtggctcctgggggttctttcttcctctgctgaaggaataaattgctccttgacattgtagagcttctggcacttggagacttgtatgaaagatggctgtgcctctgcctgtctcccccaccgggctgggagctctgcagagcaggaaacatgactcgtatatgtctcaggtccctgcagggccaagcacctagcctcgctcttggcaggtactcagcgaatgaatgctgtatatgttgggtgcaaagttccctacttcctgtgacttcagctctgttttacaataaaatcttgaaaatgcctaaaaaaaaaaaaaaaaaaaaaaaaaaaaaaaaaaaaaaaaaaaaaaaaaaaa

SEQ ID NO: 9 - protein, 350 aa, Homo sapiens
MAATSLMSALAARLLQPAHSCSLRLRPFHLAAVRNEAVVISGRKLAQQIKQEVRQEVEEWVASGNKRPHLSVILVGENPASHSYVLNKTRAAAVVGINSETIMKPASISEEELLNLINKLNNDDNVDGLLVQLPLPEHIDERRICNAVSPDKDVDGFHVINVGRMCLDQYSMLPATPWGVWEIIKRTGIPTLGKNVVVAGRSKNVGMPIAMLLHTDGAHERPGGDATVTISHRYTPKEQLKKHTILADIVISAAGIPNLITADMIKEGAAVIDVGINRVHDPVTAKPKLVGDVDFEGVRQKAGYITPVPGGVGPMTVAMLMKNTIIAAKKVLRLEEREVLKSKELGVATN

SEQ ID NO: 10 - DNA, 2208 bp, Homo sapiens
  source 1..2208: mol_type = genomic DNA
ggggcctgccacgaggccgcagtataaccgcgtggcccgcgcgcgcgcttccctcccggcgcagtcaccggcgcggtctatggctgcgacttctctaatgtctgctttggctgcccggctgctgcagcccgcgcacagctgctcccttcgccttcgccctttccacctcgcggcagttcgaaatgaagctgttgtcatttctggaaggaaactggcccagcagatcaagcaggaagtgcggcaggaggtagaagagtgggtggcctcaggcaacaaacggccacacctgagtgtgatcctggttggcgagaatcctgcaagtcactcctatgtcctcaacaaaaccagggcagctgcagttgtgggaatcaacagtgagacaattatgaaaccagcttcaatttcagaggaagaattgttgaatttaatcaataaactgaataatgatgataatgtagatggcctccttgttcagttgcctcttccagagcatattgatgagagaaggatctgcaatgctgtttctccagacaaggatgttgatggctttcatgtaattaatgtaggacgaatgtgtttggatcagtattccatgttaccggctactccatggggtgtgtgggaaataatcaagcgaactggcattccaaccctagggaagaatgtggttgtggctggaaggtcaaaaaacgttggaatgcccattgcaatgttactgcacacagatggggcgcatgaacgtcccggaggtgatgccactgttacaatatctcatcgatatactcccaaagagcagttgaagaaacatacaattcttgcagatattgtaatatctgctgcaggtattccaaatctgatcacagcagatatgatcaaggaaggagcagcagtcattgatgtgggaataaatagagttcacgatcctgtaactgccaaacccaagttggttggagatgtggattttgaaggagtcagacaaaaagctgggtatatcactccagttcctggaggtgttggccccatgacagtggcaatgctaatgaagaataccattattgctgcaaaaaaggtgctgaggcttgaagagcgagaagtgctgaagtctaaagagcttggggtagccactaattaactactgtgtcttctgtgtcacaaacagcactccaggccagctcaagaagcaaagcaggccaatagaaatgcaatatttttaatttattctactgaaatggtttaaaatgatgccttgtatttattgaaagcttaaatgggtgggtgtttctgcacatacctctgcagtacctcaccagggagcattccagtatcatgcagggtcctgtgatctagccaggagcagccattaacctagtgattaatatgggagacattaccatatggaggatggatgcttcactttgtcaagcacctcagttacacattcgccttttctaggattgcatttcccaagtgctattgcaataacagttgatactcattttaggtaccaaaccttttgagttcaactgatcaaaccaaaggaaaagtgttgctagagaaaattagggaaaaggtgaaaaagaaaaaatggtagtaattgagcagaaaaaaattaatttatatatgtattgattggcaaccagatttatctaagtagaactgaattggctaggaaaaaagaaaaactgcatgttaatcattttcctaagctgtccttttgaggcttagtcagtttattgggaaaatgtttaggattattccttgctattagtactcattttatgtatgttacccttcagtaagttctccccattttagttttctaggactgaaaggattcttttctacattatacatgtgtgttgtcatatttggcttttgctatatactttaacttcattgttaaatttttgtattgtatagtttctttggtgtatcttaaaacctatttttgaaaaacaaacttggcttgataatcatttgggcagcttgggtaagtacgcaacttacttttccaccaaagaactgtcagcagctgcctgcttttctgtgatgtatgtatcctgttgacttttccagaaattttttaagagtttgagttactattgaatttaatcagactttctgattaaagggttttctttcttttttaataaaacacatctgtctggtatggtatgaatttctgaaaaaaaaaaaaaaaaaaaaaaa

SEQ ID NO: 11 - protein, 287 aa, Homo sapiens
ERCCRCCCRCPCCGQGPRSGCKIPAGDARVRDGAGGSVHLCALFGLLPRVWLERFYPCLWPLVPGKGQCRPGDSMATNNSTQKVQKETQGRFHLLGDPSRNNCSLSIRDARRRDNGSYFFWVARRRTKFSYKYSPLSVYVTALTHRPDILIPEFLKSGHPSNLTCSVPWVCEQGTPPIFSWMSAAPTSLGPRTLHSSELTIIPRPQDHGTNLICQVTFPGAGVTTERTIQLSVSWKSGTVEEVVVLAVGVVAVKILLLCLCLIILSFHKKKAVRAVEVEENVYAVMG

SEQ ID NO: 12 - DNA, 2251 bp, Homo sapiens
  source 1..2251: mol_type = genomic DNA
ccaagatctcatgctcctccccacagccctcttctctgctcacacaggaagcccaggaagcctctgcctcagagatgctgccgctgctgctgccgctgcccctgctgtgggcagggccctcgctcaggatgcaagattccggctggagatgccagagtccgtgacggtgcaggagggtctgtgcatctttgtgcactgttcggtcttctacctcgagtatggctggaaagattctacccctgcttatggccactggttccgggaaggggtcagtgtagaccaggagactccagtggccacaaacaactcaactcaaaaagtgcagaaggagacccagggccgattccacctcctcggtgatccctcaaggaacaactgctccctgagcatcagagacgccaggaggagggacaacggttcatacttcttttgggtggcgagaggaagaacaaaatttagttacaaatattccccgctctctgtgtatgtgacagccctgacccacaggcccgacatcctcatcccggagttcctaaagtctggccatcccagcaacctgacctgctctgtgccctgggtctgtgagcagggaacaccccccatcttctcctggatgtcagctgcccccacctccctgggccccaggaccctccactcctcagtgctcacgatcatcccacggcctcaggaccacggcaccaacctcatctgtcaggtgacgttccccggagctggtgtgaccacggagagaaccatccagctcagtgtctcctggaaatcaggaaccgtggaagaggtggttgttttggccgtgggggtagtggctgtgaagatcctgcttctctgcctttgcctcatcatcctcaggtaagcactgcctgaagaccaaggacaggcatggggagggcagaggacatgatgctgaatcccagaatctcaatcctgggggtatgcagacagtttacgtggtcctggggccaggctggaggctgaattggtggtgagaattacacatgggccatttgtggtcagtttgtgtctgtcccaactgagggccaaatgccaggatggggagcttcctgttgtcatcaaggaagtctagacctgctcttcctccctgtgatccctccagcctctagcagggcacaggaagttgagttggctgccctttgctcccttcatgtggccacacttacaggtccttgtctcttcactcaatgtcagtttccacaagaagaaggcggtgagggcagtggaggttgaggagaatgtatatgctgtcatgggttaatctctcaggtgagtgatgtgggcctctcactcttcaacatcctgctggataactcctccacaatggcctccaggattgctctgcccatcatggccaaagttaagccaactgtcttcctcctcaaacctacttttcctgggatgtgggttcttcatcctgcagatgacaaagaggcctcatctctaaagtcagaacctgggtgtgggtctccatcttgacccccctcccttctctagatcccataaattactagctcttgtccctccttctcctaagcagggctcatcttgatgcccttttctccatcctgaccctggtcattcttgcggcctcacctcttccctgatcactgaacccttttcacctcctgcctccatctctccccaacacaggcctccagactgtacttccagatgtctcctcatccagttcctccacagtctgaatggccatgtttcctcttcattgctggagaatgaagtgcaaatgccactgcctggactgaaggcctttcacgatctgtcttctgctggactctgctcctgatcccccttctccttgcatcacccgaagtctccctacacccaccaggccaagccctctgtgattctgagactttgcatgtgtagttacttctcctgaaatggccttcctccccattcctgccaatccaggtccttatcatccttcaggttgtcttaaatgtcatccaggtgtgtgtatttttatgtaatccttgtatgatattaagcggagatgtggcatttgttcattaatttgtagacatattcagtaaccatactgaatacatataatgactatgtgccagcatttccgtatgtgcagaagttcatcaatagatatagactcaaagagctctgtcatcaagctgttgttctgaagagcagaaggatacaaataaaaagaaataagtaaaataaaaaaaaaaaaaaaaaaaaa

SEQ ID NO: 13 - protein, 790 aa, Homo sapiens
MAEQVLPQALYLSNMRKAVKIRERTPEDIFKPTNGIIHHFKTMHRYTLEMFRTCQFCPQFREIIHKALIDRNIQATLESQKKLNWCREVRKLVALKTNGDGNCLMHATSQYMWGVQDTDLVLRKALFSTLKETDTRNFKFRWQLESLKSQEFVETGLCYDTRNWNDEWDNLIKMASTDTPMARSGLQYNSLEEIHIFVLCNILRRPIIVISDKMLRSLESGSNFAPLKVGGIYLPLHWPAQECYRYPIVLGYDSHHFVPLVTLKDSGPEIRAVPLVNRDRGRFEDLKVHFLTDPENEMKEKLLKEYLMVIEIPVQGWDHGTTHLINAAKLDEANLPKEINLVDDYFELVQHEYKKWQENSEQGRREGHAQNPMEPSVPQLSLMDVKCETPNCPFFMSVNTQPLCHECSERRQKNQNKLPKLNSKPGPEGLPGMALGASRGEAYEPLAWNPEESTGGPHSAPPTAPSPFLFSETTAMKCRSPGCPFTLNVQHNGFCERCHNARQLHASHAPDHTRHLDPGKCQACLQDVTRTFNGICSTCFKRTTAEASSSLSTSLPPSCHQRSKSDPSRLVRSPSPHSCHRAGNDAPAGCLSQAARTPGDRTGTSKCRKAGCVYFGTPENKGFCTLCFIEYRENKHFAAASGKVSPTASRFQNTIPCLGRECGTLGSTMFEGYCQKCFIEAQNQRFHEAKRTEEQLRSSQRRDVPRTTQSTSRPKCARASCKNILACRSEELCMECQHPNQRMGPGAHRGEPAPEDPPKQRCRAPACDHFGNAKCNGYCNECFQFKQMYG

SEQ ID NO: 14 - DNA, 4705 bp, Homo sapiens
  source 1..4705: mol_type = genomic DNA
ctttggaaagtcccgtggaaatccccgggcctacaacccgcatacaactgaaacggggcaaagcagactgcgcagtctgcagtcttcgtggcgggccaagcgagcttggagcccgcgggggcggagcggtgagagcggccgccaagagagatcacacccccagccgaccctgccagcgagcgagcccgaccccaggcgtccatggagcgtcgcctccgcccggtccctgccccgacccccgcctgcggcgcgctcctgccttgaccaggacttgggactttgcgaaaggatcgcggggcccggagaggtgttggagagcacaatggctgaacaagtccttcctcaggctttgtatttgagcaatatgcggaaagctgtgaagatacgggagagaactccagaagacatttttaaacctactaatgggatcattcatcattttaaaaccatgcaccgatacacactggaaatgttcagaacttgccagttttgtcctcagtttcgggagatcatccacaaagccctcatcgacagaaacatccaggccaccctggaaagccagaagaaactcaactggtgtcgagaagtccggaagcttgtggcgctgaaaacgaacggtgacggcaattgcctcatgcatgccacttctcagtacatgtggggcgttcaggacacagacttggtactgaggaaggcgctgttcagcacgctcaaggaaacagacacacgcaactttaaattccgctggcaactggagtctctcaaatctcaggaatttgttgaaacggggctttgctatgatactcggaactggaatgatgaatgggacaatcttatcaaaatggcttccacagacacacccatggcccgaagtggacttcagtacaactcactggaagaaatacacatatttgtcctttgcaacatcctcagaaggccaatcattgtcatttcagacaaaatgctaagaagtttggaatcaggttccaatttcgcccctttgaaagtgggtggaatttacttgcctctccactggcctgcccaggaatgctacagataccccattgttctcggctatgacagccatcattttgtacccttggtgaccctgaaggacagtgggcctgaaatccgagctgttccacttgttaacagagaccggggaagatttgaagacttaaaagttcactttttgacagatcctgaaaatgagatgaaggagaagctcttaaaagagtacttaatggtgatagaaatccccgtccaaggctgggaccatggcacaactcatctcatcaatgccgcaaagttggatgaagctaacttaccaaaagaaatcaatctggtagatgattactttgaacttgttcagcatgagtacaagaaatggcaggaaaacagcgagcaggggaggagagaggggcacgcccagaatcccatggaaccttccgtgccccagctttctctcatggatgtaaaatgtgaaacgcccaactgccccttcttcatgtctgtgaacacccagcctttatgccatgagtgctcagagaggcggcaaaagaatcaaaacaaactcccaaagctgaactccaagccgggccctgaggggctccctggcatggcgctcggggcctctcggggagaagcctatgagcccttggcgtggaaccctgaggagtccactggggggcctcattcggccccaccgacagcacccagcccttttctgttcagtgagaccactgccatgaagtgcaggagccccggctgccccttcacactgaatgtgcagcacaacggattttgtgaacgttgccacaacgcccggcaacttcacgccagccacgccccagaccacacaaggcacttggatcccgggaagtgccaagcctgcctccaggatgttaccaggacatttaatgggatctgcagtacttgcttcaaaaggactacagcagaggcctcctccagcctcagcaccagcctccctccttcctgtcaccagcgttccaagtcagatccctcgcggctcgtccggagcccctccccgcattcttgccacagagctggaaacgacgcccctgctggctgcctgtctcaagctgcacggactcctggggacaggacggggacgagcaagtgcagaaaagccggctgcgtgtattttgggactccagaaaacaagggcttttgcacactgtgtttcatcgagtacagagaaaacaaacattttgctgctgcctcagggaaagtcagtcccacagcgtccaggttccagaacaccattccgtgcctggggagggaatgcggcacccttggaagcaccatgtttgaaggatactgccagaagtgtttcattgaagctcagaatcagagatttcatgaggccaaaaggacagaagagcaactgagatcgagccagcgcagagatgtgcctcgaaccacacaaagcacctcaaggcccaagtgcgcccgggcctcctgcaagaacatcctggcctgccgcagcgaggagctctgcatggagtgtcagcatcccaaccagaggatgggccctggggcccaccggggtgagcctgcccccgaagacccccccaagcagcgttgccgggcccccgcctgtgatcattttggcaatgccaagtgcaacggctactgcaacgaatgctttcagttcaagcagatgtatggctaaccggaaacaggtgggtcacctcctgcaagaagtggggcctcgagctgtcagtcatcatggtgctatcctctgaacccctcagctgccactgcaacagtgggcttaagggtgtctgagcaggagaggaaagataagctcttcgtggtgcccacgatgctcaggtttggtaacccgggagtgttcccaggtggccttagaaagcaaagcttgtaactggcaagggatgatgtcagattcagcccaaggttcctcctctcctaccaagcaggaggccaggaacttctttggacttggaaggtgtgcggggactggccgaggcccctgcaccctgcgcatcaggactgcttcatcgtcttggctgagaaagggaaaagacacacaagtcgcgtgggttggagaagccagagccattccacctcccctcccccagcatctctcagagatgtgaagccagatcctcatggcagcgaggccctctgcaagaagctcaaggaagctcagggaaaatggacgtattcagagagtgtttgtagttcatggtttttccctacctgcccggttcctttcctgaggacccggcagaaatgcagaaccatccatggactgtgattctgaggctgctgagactgaacatgttcacattgacagaaaaacaagctgctctttataatatgcaccttttaaaaaattagaatattttactgggaagacgtgtaactctttgggttattactgtctttacttctaaagaagttagcttgaactgaggagtaaaagtgtgtacatatataatatacccttacattatgtatgagggatttttttaaattatattgaaatgctgccctagaagtacaataggaaggctaaataataataacctgttttctggttgttgttggggcatgagcttgtgtatacactgcttgcataaactcaaccagctgcctttttaaagggagctctagtcctttttgtgtaattcactttatttattttattacaaacttcaagattatttaagtgaagatatttcttcagctctggggaaaatgccacagtgttctcctgagagaacatccttgctttgagtcaggctgtgggcaagttcctgaccacagggagtaaattggcctctttgatacacttttgcttgcctccccaggaaagaaggaattgcatccaaggtatacatacatattcatcgatgtttcgtgcttctccttatgaaactccagctatgtaataaaaaactatactctgtgttctgttaatgcctctgagtgtcctacctccttggagatgagatagggaaggagcagggatgagactggcaatggtcacagggaaagatgtggccttttgtgatggttttattttctgttaacactgtgtcctgggggggctgggaagtcccctgcatcccatggtaccctggtattgggacagcaaaagccagtaaccatgagtatgaggaaatctctttctgttgctggcttacagtttctctgtgtgctttgtggttgctgtcatatttgctctagaagaaaaaaaaaaaaggaggggaaatgcattttccccagagataaaggctgccattttgggggtctgtacttatggcctgaaaatatttgtgatccataactctacacagcctttactcatactattaggcacactttccccttagagccccctaagtttttcccagacgaatctttataatttctttccaaagataccaaataaacttcagtgttttcatctaattctcttaaagttgatatcttaatattttgtgttgatcattatttccattcttaatgtgaaaaaaagtaattatttatacttattataaaaagtatttgaaatttgcacatttaattgtccctaatagaaagccacctattctttgttggatttcttcaagtttttctaaataaatgtaacttttcacaagagtcaacattaaaaaataaattatttaagaacagaaaaaaaaaaaaaaaa

SEQ ID NO: 15 - protein, 129 aa, Homo sapiens
MARGSLRRLLRLLVLGLWLALLRSVAGEQAPGTAPCSRGSSWSADLDKCMDCASCRARPHSDFCLGCAAAPPAPFRLLWPILGGALSLTFVLGLLSGFLVWRRCRRREKFTTPIEETGGEGCPAVALIQ

SEQ ID NO: 16 - DNA, 1048 bp, Homo sapiens
  source 1..1048: mol_type = genomic DNA
aaggcgggggcgggggcggggcggcggccgtgggtccctgccggccggcggcgggcgcagacagcggcgggcgcaggacgtgcactatggctcggggctcgctgcgccggttgctgcggctcctcgtgctggggctctggctggcgttgctgcgctccgtggccggggagcaagcgccaggcaccgccccctgctcccgcggcagctcctggagcgcggacctggacaagtgcatggactgcgcgtcttgcagggcgcgaccgcacagcgacttctgcctgggctgcgctgcagcacctcctgcccccttccggctgctttggcccatccttgggggcgctctgagcctgaccttcgtgctggggctgctttctggctttttggtctggagacgatgccgcaggagagagaagttcaccacccccatagaggagaccggcggagagggctgcccagctgtggcgctgatccagtgacaatgtgccccctgccagccggggctcgcccactcatcattcattcatccattctagagccagtctctgcctcccagacgcggcgggagccaagctcctccaaccacaaggggggtggggggcggtgaatcacctctgaggcctgggcccagggttcaggggaaccttccaaggtgtctggttgccctgcctctggctccagaacagaaagggagcctcacgctggctcacacaaaacagctgacactgactaaggaactgcagcatttgcacaggggaggggggtgccctccttcctagaggccctgggggccaggctgacttggggggcagacttgacactaggccccactcactcagatgtcctgaaattccaccacgggggtcaccctggggggttagggacctatttttaacactagggggctggcccactaggagggctggccctaagatacagacccccccaactccccaaagcggggaggagatatttattttggggagagtttggaggggagggagaatttattaataaaagaatctttaactttaaaaaaaaaaaaaaaaaa

SEQ ID NO: 17 - protein, 706 aa, Homo sapiens
MEENPTLESEAWGSSREWLAPREARGGPSLSSVLNELPSAATLRYRDPGVLPWGALEEEEEDGGRSRKAFTEVTQTELQDPHPSRELPWPMQARRAHRQRNASRDQVVYGSGTKTDRWARLLRRSKEKTKEGLRSLQPWAWTLKRIGGQFGAGTESYFSLLRFLLLLNVLASVLMACMTLLPTWLGGAPPGPPGPDISSPCGSYNPHSQGLVTFATQLFNLLSGEGYLEWSPLFYGFYPPRPRLAVTYLCWAFAVGLICLLLILHRSVSGLKQTLLAESEALTSYSHRVFSAWDFGLCGDVHVRLRQRIILYELKVELEETVVRRQAAVRTLGQQARVWLVRVLLNLLVVALLGAAFYGVYWATGCTVELQEMPLVQELPLLKLGVNYLPSIFIAGVNFVLPPVFKLIAPLEGYTRSRQIVFILLRTVFLRLASLVVLLFSLWNQITCGGDSEAEDCKTCGYNYKQLPCWETVLGQEMYKLLLFDLLTVLAVALLIQFPRKLLCGLCPGALGRLAGTQEFQVPDEVLGLIYAQTVVWVGSFFCPLLPLLNTVKFLLLFYLKKLTLFSTCSPAARTFRASAANFFFPLVLLLGLAISSVPLLYSIFLIPPSKLCGPFRGQSSIWAQIPESISSLPETTQNFLFFLGTQAFAVPLLLISSILMAYTVALANSYGRLISELKRQRETEAQNKVFLARRAVALTSTKPAL

SEQ ID NO: 18 - DNA, 2477 bp, Homo sapiens
  source 1..2477: mol_type = genomic DNA
ctgagaaaccacaggaagtgtaccttactccctccgggccacctgctggccaggtacacacctgcccctggcccctcccttacctggggcagtgtctgcctggtggccactagagacagcccagcctggggccatggaagaaaacccgaccttggaatcagaagcctggggctcctctagggagtggctggccccccgggaggccagaggaggcccatcgctgtcttctgtgctgaacgagctgcccagtgctgccacccttcggtaccgagaccctggggtgctgccttggggggcgctggaggaggaggaggaggatggaggaaggagcagaaaggccttcacagaagtcacccagacagagctgcaggaccctcacccttcccgggaactgccctggcccatgcaggccagacgggcacacaggcaaagaaatgccagcagggaccaggtggtctatggctctggaactaagacggaccgatgggcgcggctacttcggaggtccaaggagaaaacaaaggaaggcttgcgaagcctgcagccctgggcgtggacactgaagaggatcgggggccagtttggcgccggcacggagtcctacttctccctgctgcgcttcctgctccttcttaacgtgctggcctctgtgctcatggcctgcatgacgctgctgcccacctggttgggaggcgctcccccaggccctcccggccccgacatctcctcgccctgcggctcctataacccccactcccagggcctggtcacctttgccacccagctcttcaacttgctctcgggtgagggttacctggaatggtcccctctcttctatggcttctacccgccccgcccacgcctggcggtcacctacctgtgctgggcctttgccgttggcctcatctgcctcctgctcatcctgcatcgctcggtgtctgggctgaagcagacactgctggcggagtccgaggctctgaccagctacagccaccgggtgttctcggcctgggacttcggtctctgcggggacgtccacgtgcggctgcgccagcgcatcatcttgtacgaattaaaggtggagctggaggagacagtggtgcggcgccaggctgcggtgcggacgctgggccagcaagccagggtttggttggtgcgggtgctgctcaacctgctggtggtcgcgctcctgggggcagccttctatggcgtctactgggctacggggtgcaccgtggagctgcaggagatgccccttgtccaggagttgccactgctgaagcttggggtgaattaccttccgtccatcttcatcgctggggtcaattttgtgctgccgcccgtgttcaagctcattgctccactggagggctacactcggagtcgccagatcgtttttatcctgctcaggaccgtgtttcttcgcctcgcctccctggtggtcctgctcttctctctctggaatcagatcacttgtgggggcgactccgaggctgaggactgcaaaacctgtggctacaattacaaacaacttccgtgctgggagactgtcctgggccaggaaatgtacaaacttctgctctttgatctgctgactgtcttggcagtcgcgctgctcatccagtttcctagaaagctcctctgtggcctctgtcctggggcgctgggtcgtctggcggggacccaagagttccaggtgcccgacgaggtgctggggctcatctacgcgcagacggtggtctgggtggggagttttttctgccctttactgcccctgcttaacacggtcaagttcctgctgcttttctacctgaagaagcttaccctcttctccacctgctccccggctgcccgcaccttccgggcctccgcggcgaatttctttttccccttggtccttctcctgggtctggccatctccagcgttcccctgctttacagcatcttcctgatcccgccttctaagctgtgtggtccattccgggggcagtcgtccatctgggcccagatccctgagtctatttccagcctccctgagaccacccagaatttcctcttcttcctggggacccaggcttttgctgtgccccttctgctgatctccagcatcctgatggcgtacactgtggctctggctaactcctacggacgcctcatctctgagctcaaacgtcagagagagacggaggcgcagaataaagtcttcctggcacggcgcgctgtggcgctgacctccaccaaaccggctctttgacccccgcagcccacgtcccgctttcagaccccaggcccattgtaagcctaggtcacaacatctgtaaactaggagaactggagaagactccacgcccttccagctttggtatctggagatttccagggcccctcgccgccacgtccctgactctcgggtgatcttccttgtatcaataaatacagccgaggttgctgagcgcgctttgaaaaaaaaaaaaaaa

(19) United States
(12) Patent Application Publication
(10) Pub. No.: US 2025/0101517 A1
(43) Pub. Date: Mar. 27, 2025

(54) IDENTIFICATION OF UNIQUE BLOOD-BASED GENE EXPRESSION PROFILES IN CHILDREN WITH REGRESSIVE AUTISM SPECTRUM DISORDER (ASD) AND ILEOCOLITIS

(71) Applicants: Arthur KRIGSMAN, Far Rockaway, NY (US); WAKE FOREST UNIVERSITY HEALTH SCIENCES, Winston-Salem, NC (US)

(72) Inventors: Arthur KRIGSMAN, Far Rockaway, NY (US); Stephen WALKER, Winston-Salem, NC (US)

(21) Appl. No.: 18/655,068

(22) Filed: May 3, 2024

Related U.S. Application Data
(63) Continuation of application No. 18/453,144, filed on Aug. 21, 2023, now abandoned.
Application No. 18/453,144 is a continuation of application No. 17/559,979, filed on Dec. 22, 2021, now abandoned.
Application No. 17/559,979 is a continuation of application No. 16/088,359, filed on Sep. 25, 2018, now Pat. No. 11,248,268.
(60) Provisional application No. 62/315,391, filed on Mar. 30, 2016.

(51) Int. Cl. C12Q 1/6883; C12Q 1/68; G01N 33/48; G01N 33/68

(52) U.S. Cl. CPC C12Q 1/6883; C12Q 1/68; G01N 33/48; G01N 33/689; G01N 33/6893; G01N 33/6896; C12Q 2600/158; G01N 2800/06

(57) ABSTRACT

The invention provides compositions and methods for identifying autism and autism spectrum disorders in humans. The invention also includes compositions and methods for identifying unique blood-based gene expression profiles in children with regressive autism spectrum disorder (ASD) and ilcocolitis.

### CROSS REFERENCE TO RELATED APPLICATIONS

The present application is a continuation of U.S. patent application Ser. No. 18/453,144, filed Aug. 21, 2023, which is a continuation of U.S. patent application Ser. No. 17/559,979, filed Dec. 22, 2021, now abandoned, which is a continuation of

U.S. patent application Ser. No. 16/088,359, filed Sep. 25, 2018, now U.S. Pat. No. 11,248,268, which is a 35 U.S.C. § 371 national phase application from, and claims priority to, International Application No. PCT/US2017/024037, filed Mar. 24, 2017, and published under PCT Article 21(2) in English, which claims priority under 35 U.S.C. § 119 (e) to U.S. Provisional Patent Application No. 62/315,391, filed Mar. 30, 2016, all of which applications are incorporated by reference herein in their entireties.

### SEQUENCE LISTING

The present application contains a Sequence Listing which has been submitted in XML format via Patent Center and is hereby incorporated by reference in its entirety. Said XML file, created on May 3, 2024, is named 205286_7006US4_SequenceListing.xml and is 49,465 bytes in size.

### BACKGROUND OF THE INVENTION

Gastrointestinal (GI) symptoms are a common comorbidity in children with autism (McElhanon et al., 2014, Pediatrics 133:872-883; Chaidez et al., 2014, J Autism Dev Disord 44:1117-1127; Gorrindo et al., 2012, Autism Res 5:101-108; Buie et al., 2010, Pediatrics 125 Suppl 1: S1-18; Bauman et al., 2010, Neurotherapeutics 7(3):320-327; Smith et al., 2009, Autism 13:343-355; Valicenti-McDermott et al., 2006, J Dev Behav Pediatr 27(2 Suppl): S128-136; Horvath et al., 2002, Current Opinion in Ped 14: 583-587; Krigsman et al., 2010, Autism Insights 1:1-11; Horvath et al., 1999, Journal of Pediatrics 135(5):559-563) occurring at a frequency as high as 85% (Gorrindo et al., 2012, Autism Res 5:101-108; Valicenti-McDermott et al., 2006, J Dev Behav Pediatr 27 (2 Suppl): S128-136). Symptoms most often reported are constipation, diarrhea, abdominal pain, abdominal distention, and food intolerances (McElhanon et al., 2014, Pediatrics 133:872-883; Buie et al., 2010, Pediatrics 125 Suppl 1: S1-18; Bauman et al., 2010, Neurotherapeutics 7(3): 320-327; Krigsman et al., 2010, Autism Insights 1:1-11). Following examination of endoscopically obtained gastrointestinal tissue in GI symptomatic autism spectrum disorder (ASD) children, numerous pathologic etiologies that may explain these GI symptoms have been described including gastroesophageal reflux (GERD) (Horvath et al., 1999, Journal of Pediatrics 135(5):559-563), cosinophilic esophagitis (Jarocka-Cyrta et al., 2011, J Autism Dev Disord 41(3):372-374), food allergies (Lyall et al., 2015, Autism Res 8(5):567-574), and inflammatory bowel disease (IBD) (Doshi-Velez et al., 2015, Bowel Dis. 21(10):2281-2288). IBD has been shown to occur with greater frequency in ASD than in typically-developing children (Doshi-Velez et al., 2015, Bowel Dis. 21(10):2281-2288).

In addition to these diagnoses found also in non-ASD children, GI symptoms in ASD have also been attributed to a unique variant of inflammatory bowel disease seen only in children with ASD (Krigsman et al., 2010, Autism Insights 1:1-11; Wakefield et al., 1998, Lancet 351(9103):637-41; Wakefield et al., 2000, American Journal of Gastroenterology 95(9):2285-2295; Gonzalez et al., 2005, Arch Venez Pueric Pediat 69: 19-25). Distinguishing features of this ASD-associated inflammatory bowel disease have been described in the stomach (Torrente et al., 2004, Am J Gastroenterol 4:598-605), small intestine (Torrente et al., 2002, Mol Psychiatry 7:375-382; Ashwood et al., 2004, J Clin Immunol 24:664-673; Ashwood et al., 2006, Neuroimmunol 173:126-134; Walker et al., 2013, PLOS One 8:e58058), and colon (Ashwood et al., 2004, J Clin Immunol 24:664-673; Walker et al., 2013, PLOS One 8:e58058; Furlano et al., 2001, J Pediatr 138:366-372) of GI symptomatic ASD children undergoing diagnostic endoscopy in which unique cellular, immunohistochemical and molecular properties have been characterized. ASD-associated ilcocolitis has been shown to occur at a frequency of up to 70% in GI symptomatic children undergoing diagnostic ilcocolonoscopy (Krigsman et al., 2010, Autism Insights 1:1-11) and represents a much higher prevalence than the already increased incidence of “classic” IBD seen in these children (Doshi-Velez et al, 2015, Inflamm Bowel Dis. 21(10): 2281-228813). The clinical significance of identification and treatment of ASD-associated ilcocolitis and IBD extends beyond simple resolution of troubling chronic GI symptoms, especially in light of the association of GI symptoms with extremes of ASD behavioral features (Chaidez et al., 2014, J Autism Dev Disord 44:1117-1127; Gorrindo et al., 2012, Autism Res 5:101-108; Mazurek et al., 2013, J Abnorm Child Psychol 41(1):165-176; Maenner et al., 2012, J Autism Dev Disord 42(7):1520-1525; Adams et al., 2011, BMC Gastroenterology 11:22; Nikolov et al., 2009, J Autism Dev Disord 39(3):405-41323-26). As such, treatment of symptomatic underlying GI pathology, regardless of its etiology, may improve not only the presenting GI symptoms but behavioral symptoms as well.

Historically, GI symptoms presenting in the setting of ASD have often either gone unrecognized (Buie et al., 2010, Pediatrics 125 Suppl 1: S1-18) or been treated empirically. Empiric treatment of a chronic condition such an inflammatory bowel disease typically affords only transient improvement, if any at all. Because of the significant practical difficulties inherent in performing diagnostic endoscopy in GI symptomatic children with ASD, a blood biomarker that could reliably identify those GI symptomatic ASD children most likely to have an IBD, and thus be most likely to benefit from diagnostic endoscopy, would be of enormous clinical value.

Currently, a formal ASD diagnosis is based upon meeting DSMV criteria and unfortunately the diagnosis is often given at a time beyond the ideal “developmental window of opportunity” for commencing time-sensitive, maximally effective, interventions. For this reason the search for diagnostic biomarkers capable of identifying at-risk children as early as possible has become a priority (Loth et al., 2016, Nat Rev Drug Discov 15(1):70-73; Ruggeri et al., 2014, Psychopharmacology (Berl) 231(6):1201-1216; Pierce et al., 2009, Ann Clin Psychiatry 21(3):132-147). There are numerous efforts currently underway to identify diagnostically relevant biomolecules (e.g. microRNAs (Vasu et al., 2014, Mol Autism 5(40):1-9), mitochondrial DNA (Chen et al., 2015, BMC Psychiatry 15(50):1-7), cytokines (Jyonouchi et al., 2014, J Neuroinflammation 11(187):1-13), mRNAs (Pramparo et al., 2015, JAMA Psychiatry 72(4):386-394; Yang et al., 2015, Neuroscience 284:290-296; Segura et al., 2015, Neurogenetics 16(2):123-131; Kong et al., 2013, Neurogenetics 14:143-152; Campbell et al., 2013, BMC Medical Genomics 6(34):1-15; Kong et al., 2012, PLOS One 7(12):1-13; Glatt et al., 2012, J Am Acad Child Adolesc Psychiatry 51(9):934-944; Taurines et al., 2011, World J Biol Psychiatry Suppl 1:104-108) in the peripheral blood of at-risk children.

Given the broad heterogeneity that is the hallmark of ASD, coupled with the understanding that earlier diagnosis and treatment provides the greatest chance for the most positive outcomes, a blood-based test to diagnose autism (and/or ASD subtypes) early on would have tremendous clinical value. A key confound in these efforts, heterogeneity (in the form of ASD subtypes) exists within the core domains (language, social interaction, and range of interests), but also within associated medical comorbidity domains (e.g. epilepsy, sleep disorders, gastrointestinal disorders, etc.).

Biomarker discovery efforts especially relevant to the area of inflammatory bowel disease are numerous. Diagnostic uncertainty resultant from clinical overlap between the two recognized types of IBD-Crohn's disease (CD) and ulcerative colitis (UC)—have inspired the numerous attempts to delineate IBD subtypes through comparisons of gene expression profiles in either mucosal biopsy tissue (e.g. Wu et al., 2006, Inflamm Bowel Dis 13(7):807-821; von Stein et al., 2008, Gastroenterology 134(7):1869-1881; von Stein et al., 2009, Expert Rev Mol Diag 9(1):7-10; Granlund et al., 2013, PLOS One 8(2):1-13] or peripheral blood (e.g., Mannick et al., 2004, Clin Immunol 112(3):247-257; Burczynski et al., 2005, J Mol Diag 8(1):51-61; Sipos et al., 2011, Dis Markers 30(1):1-17; Burakoff et al., 2011, Inflamm Bowel Dis 17(8):1719-1725; van Lierop et al., 2013, PLOS One 8(11):1-8). Moreover, these efforts are driven by the fact that getting the appropriate (and definitive) IBD diagnosis has important implications for early and successful therapeutic intervention and disease management. Because ileocolitis in ASD children shares many clinical and molecular similarities with classic pediatric IBD while at the same time having distinct clinical and molecular features, our original mucosal-based gene expression study in ASD^{IC+} children (Walker et al., 2013, PLOS One 8:e58058) was modeled after the study by von Stein (von Stein et al., 2009, Expert Rev Mol Diag 9(1):7-10) that reported a biomarker, consisting of seven transcripts, which could be used to distinguish Crohn's disease from ulcerative colitis.

The rationale for evaluating peripheral blood in search of disease biomarkers begins with the answer to an important fundamental question: “To what extent does expression in WBCs (white blood cells) reflect expression in other organ systems?” (Kohane et al., 2012, Bioinformatics 28(4):538-545). More specifically, if one is investigating, for example, a neuropsychiatric disease with a known pathophysiological signature in the brain, can one use the correlation/overlap between brain and blood gene expression to derive a peripheral blood-based biomarker that will faithfully identify affected individuals? To answer that question, several groups have looked specifically at the overlap of gene expression in the blood and brain tissue from diseased and control individuals (Tsuang et al., 2005, Am J Genet B Neuropsychiatr Genet 133B(1):1-5; Glatt et al., 2005, Proc Natl Acad Sci USA 102(43):15533-15538; Sullivan et al., 2006, Am J Genet B Neuropsychiatr Genet 141B(3):261-268; Rollins et al., 2010, Am J Genet B Neuropsychiatr Genet 153B(3):919-936). In fact, a recent review of eight brain/blood gene expression studies found that between 35% and 80% of known transcripts are found in both tissues and estimates of correlated (cross-tissue) expression levels ranged from 0.25-0.64, with the higher correlation found, not surprisingly, among specific subsets of genes (Tylee et al., 2013, Am J Genet B Neuropsychiatr Genet 162B(3):595-603). One of the studies in particular that measured gene expression in cadaveric brain tissue from patients with schizophrenia (SZ) and compared profiles to gene expression in peripheral blood from living SZ patients found (and validated) a strong SZ biomarker candidate gene (SELENBP1) (Glatt et al., 2005, Proc Natl Acad Sci USA 102(43):15533-15538). The authors concluded that “the identification of valid peripheral biomarkers for SZ may ultimately facilitate early identification, intervention, and prevention efforts” (Glatt et al., 2005, Proc Natl Acad Sci USA 102(43):15533-15538).

A major limitation of these types of studies has always been the quantity and quality of human banked tissue available for study (Cai et al., 2010, BMC Genomics 11(589):1-15). A second important confound is that the brain gene expression data (often from brain bank tissues) and the blood gene expression data (often from living donors) typically do not come from the same individuals. Consequently, these types of studies—whose goal is to identify peripheral biomarkers through the evaluation and comparison of diseased organ tissues and peripheral blood-are rarely able to be performed optimally due to the difficulty in obtaining relevant tissues for analysis.

Therefore, there is a need in the art for blood-based methods of diagnosing ASD, and GI disorders in children with ASD. The present invention satisfies this unmet need.

### SUMMARY OF THE INVENTION

In one aspect the invention comprises a method of treating a subject having a gastrointestinal disorder, the method comprising administering a treatment for gastrointestinal disorder to a pre-selected subject, wherein the subject is pre-selected by detecting an alteration in the level of a polypeptide or polynucleotide of at least one biomarker selected from the group consisting of FCER1A, CYP2S1, TMC4, IL1RN, TNFAIP3, CENPE, MTHFD2, and SIGLEC17P relative to a reference in a blood sample obtained from the subject.

In another aspect the invention comprises a method of diagnosing a gastrointestinal disorder in a subject, the method comprising measuring the level of a polypeptide or polynucleotide of at least one biomarker selected from the group consisting of FCER1A, CYP2S1, TMC4, IL1RN, TNFAIP3, CENPE, MTHFD2, and SIGLEC17P in a blood sample from the subject, wherein an alteration in the level of the polypeptide or polynucleotide relative to a reference indicates that the subject has a gastrointestinal disorder.

In some embodiments the subject is pre-selected by further detecting an alteration in the level of TNFRSF12A polypeptide or polynucleotide.

In some embodiments, the invention further comprises measuring the level of TNFRSF12A polypeptide or polynucleotide.

In some embodiments the subject is pre-selected by detecting an alteration in the level of a IL1RN polypeptide or polynucleotide, MTHFD2 polypeptide or polynucleotide, and SIGLEC17P polypeptide or polynucleotide.

In some embodiments the method comprises measuring the level of a IL1RN polypeptide or polynucleotide, MTHFD2 polypeptide or polynucleotide, and SIGLEC17P polypeptide or polynucleotide.

In some embodiments the alteration comprises an increase in the level of TMC4 polypeptide or polynucleotide, an increase in the level of TNFAIP3 polypeptide or polynucleotide, a decrease in the level of FCER1A polypeptide or polynucleotide, a decrease in the level of CYP2S1 polypeptide or polynucleotide, a decrease in the level of TNFRSF12A polypeptide or polynucleotide, a decrease in the level of IL1RN polypeptide or polynucleotide, a decrease in the level of CENPE polypeptide or polynucleotide, a decrease in the level of MTHFD2 polypeptide or polynucleotide, or a decrease in the level of SIGLEC17P polypeptide or polynucleotide.

In some embodiments the gastrointestinal disorder is ileocolitis, ileitis, colitis, enteritis, duodenitis, gastritis, and/or esophagitis.

In some embodiments the subject is a child.

In some embodiments the subject has an autism spectrum disorder.

In some embodiments measuring comprises PCR assays or microarrays.

In some embodiments the method further comprises a step of using the results obtained from the diagnostic assay to selecting or administering a treatment.

In some embodiments the treatment comprises corticosteroids, immunomodulators, 5-aminosalicylic acid preparations, cytokine specific antagonists, dietary restrictions, antimicrobials, probiotics, and/or supplemental digestive enzymes.

In another aspect the invention comprises a method of diagnosing autism spectrum disorder (ASD) in a subject, the method comprising measuring the level of a polypeptide or polynucleotide of at least one biomarker selected from the group consisting of FCER1A, CYP2S1, TMC4, IL1RN, TNFAIP3, CENPE, MTHFD2, and SIGLEC17P in a blood sample from the subject, wherein an alteration in the level of the polypeptide or polynucleotide relative to a reference indicates that the subject has autism spectrum disorder.

In some embodiments the method further comprises measuring the level of TNFRSF12A polypeptide or polynucleotide.

In some embodiments the method comprises measuring the level of a polypeptide or polynucleotide of biomarkers IL1RN, MTHFD2, and SIGLEC17P.

In some embodiments the alteration comprises an increase in the level of TMC4 polypeptide or polynucleotide, an increase in the level of TNFAIP3 polypeptide or polynucleotide, a decrease in the level of FCER1A polypeptide or polynucleotide, a decrease in the level of CYP2S1 polypeptide or polynucleotide, a decrease in the level of TNFRSF12A polypeptide or polynucleotide, a decrease in the level of IL1RN polypeptide or polynucleotide, a decrease in the level of CENPE polypeptide or polynucleotide, a decrease in the level of MTHFD2 polypeptide or polynucleotide, or a decrease in the level of SIGLEC17P polypeptide or polynucleotide.

In some embodiments the autism spectrum disorder is autism.

In some embodiments the subject is a child.

In some embodiments the measuring comprises PCR assays or microarrays.

In some embodiments the method further comprises a step of using the results obtained from the diagnostic assay to selecting or administering a treatment.

In some embodiments the treatment comprises corticosteroids, immunomodulators, 5-aminosalicylic acid preparations, cytokine specific antagonists, dietary restrictions, antimicrobials, probiotics, and/or supplemental digestive enzymes.

In another aspect the invention comprises a kit for the diagnosis of a gastrointestinal and/or autism spectrum disorder, the kit comprising at least one agent capable of specifically binding or hybridizing to a polypeptide or polynucleotide of a biomarker selected from the group consisting of FCER1A, CYP2S1, TMC4, IL1RN, TNFAIP3, CENPE, MTHFD2, or SIGLEC17P, and directions for using the agent for the diagnosis of a gastrointestinal and/or autism spectrum disorder.

In some embodiments the kit comprises an agent capable of specifically binding or hybridizing to a polypeptide or polynucleotide of TNFRSF12A.

In some embodiments the kit comprises an agent capable of specifically binding or hybridizing to a polypeptide or polynucleotide of IL1RN, an agent capable of specifically binding or hybridizing to a polypeptide or polynucleotide of MTHFD2, and an agent capable of specifically binding or hybridizing to a polypeptide or polynucleotide of SIGLEC17P.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1 is a set of plots showing a comparison of gene expression profiles from inflamed terminal ileum tissue from ASD patients compared to non-inflamed TI tissue from TD controls (top panel) and corresponding profiles in colonic tissue (bottom panel) at p=0.001 using principal component analysis (teal/dark grey=ASD-TI inflamed; yellow/light grey=TD-TI non-inflamed; violet/dark grey=ASD colon inflamed; yellow/light grey=TD colon non-inflamed).

FIG. 2 is a diagram showing differentially-expressed transcripts in two independent studies. Whole genome gene expression was measured in inflamed ileocolonic tissue (either terminal ileum or colon) from ASD patients with ileocolitis and compared to the corresponding non-inflamed tissue from non-ASD controls in two separate studies. The overlap in expression profiles is shown in FIG. 2 (TI 1=terminal ileum data from the first study (25); TI 2=terminal ileum data from the second study; colon 1=colon data from the first study; colon 2=colon data from the second study).

FIG. 3 is a plot showing a comparison of gene expression profiles in peripheral blood from ASD patients with inflamed ileocolonic tissue compared to peripheral blood gene expression from TD controls without ileocolonic inflammation at p=0.001 using principal component analysis. Red/dark grey=ASD blood; Yellow/light grey=TD blood.

FIG. 4 is a diagram showing overlapping gene expression. Genes that were uniquely differentially-expressed in inflamed gastrointestinal (GI) tissue from ASD patients in two separate studies and the corresponding differential gene expression in blood from all of the cases and controls (from the second study only) were compared to identify those DETs that occur in both tissues.

FIG. 5 is a plot showing a receiver operating characteristic (ROC) analysis of blood. The variable selection procedure identified a linear combination of three transcripts that together yielded an area under the curve (AUC) of 0.883.

FIGS. 6A-6I are plots showing univariate ROC curves for the nine transcripts found to be differentially expressed in mucosal tissue and blood in ASD^{IC+} individuals. FIG. 6A shows an ROC curve for gene FCER1A. FIG. 6B shows an ROC curve for gene CYP2S1. FIG. 6C shows an ROC curve for gene TNFRSF12A. FIG. 6D shows an ROC curve for gene TMC4. FIG. 6E shows an ROC curve for gene IL1RN. FIG. 6F shows an ROC curve for gene TNFAIP3. FIG. 6G shows an ROC curve for gene CENPE. FIG. 6H shows an ROC curve for gene MTHFD2. FIG. 6I shows an ROC curve for gene SIGLEC17P.

### DETAILED DESCRIPTION

The present invention relates generally to diagnostic methods and markers, prognostic methods and markers, and therapy evaluators for autism. In one embodiment, the markers of the invention make up a gene expression profile unique to children (i.e., newborn to age 21) with autism spectrum disorder (ASD) who have comorbid gastrointestinal inflammation (referred herein as “ASD^{IC}”).

Previously, an intestinally derived molecular profile of histological ASD-associated ileocolitis that confirms, on a molecular level, the presence of an ASD-associated inflammatory bowel disease (“IBD”) and discriminates it from other IBDs such as Crohn's disease (“CD”) and ulcerative colitis (“UC”) was reported (Walker et al., 2013, PLOS One 8:e58058; United States Patent Publication No. 2015-0361499 A1). In the study described herein, the findings in Walker et al., 2013, PLOS One 8:e58058 were extended in an additional case/control cohort. Reported herein is the presence of a blood-based gene expression profile that reflects the presence of ASD-associated ileocolitis and provides a putative, clinically relevant, surrogate biomarker.

Accordingly, in one embodiment, the present invention relates to blood-based biomarkers of ASD^{IC}, methods for diagnosis of ASD^{IC}, methods of determining predisposition to ASD^{IC}, methods of monitoring progression/regression of ASD^{IC}, methods of assessing efficacy of compositions for treating ASD^{IC}, methods of screening compositions for activity in modulating biomarkers of ASD^{IC}, methods of treating ASD^{IC}, as well as other methods based on detection of biomarkers of ASD^{IC} in a blood sample.

In one embodiment, the markers of the invention are useful for discriminating between different inflammatory disorders including but is not limited to Crohn's disease, ulcerative colitis, inflammatory bowel disease (IBD), and ASD^{IC}.

In one embodiment, the markers of the invention diagnose ASD-associated ileocolitis.

In one embodiment, the present invention relates to biomarkers of ASD-associated ileocolitis, methods for diagnosis of ASD-associated ileocolitis, methods of determining predisposition to ASD-associated ileocolitis, methods of monitoring progression/regression of ASD-associated ileocolitis, methods of assessing efficacy of compositions for treating ASD-associated ileocolitis, methods of screening compositions for activity in modulating biomarkers of ASD-associated ileocolitis, methods of treating ASD-associated ileocolitis, as well as other methods based on biomarkers of ASD-associated ileocolitis.

The invention also provides a method for permitting refinement of disease diagnosis, disease risk prediction, and clinical management of patients associated with ASD-associated inflammatory disease. That is, the biomarkers of the invention can be used as a marker for the disease state or disease risk. For example, the presence of the selective biomarkers of the invention permits refinement of disease diagnosis, disease risk prediction, and clinical management of patients being treated with agents that are associated with a particular ASD-associated inflammatory disease.

The invention also provides a method of diagnosing, treating, and monitoring autism, even without accompanying inflammatory bowel disease.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element. “About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics which are normal or expected for one cell or tissue type, might be abnormal for a different cell or tissue type.

By “alteration” is meant an increase or decrease. An alteration may be by as little as 1%, 2%, 3%, 4%, 5%, 10%, 20%, 30%, or by 40%, 50%, 60%, or even by as much as 75%, 80%, 90%, or 100%.

“Amplification” refers to any means by which a polynucleotide sequence is copied and thus expanded into a larger number of polynucleotide sequences, e.g., by reverse transcription, polymerase chain reaction or ligase chain reaction, among others.

An “analyte”, as used herein refers to any substance or chemical constituent that is undergoing analysis. For example, an “analyte” can refer to any atom and/or molecule; including their complexes and fragment ions. The term may refer to a single component or a set of components. In the case of biological molecules/macromolecules, such analytes include but are not limited to: polypeptides, polynucleotides, proteins, peptides, antibodies, DNA, RNA, carbohydrates, steroids, and lipids, and any detectable moiety thereof, e.g. immunologically detectable fragments. In some instances, an analyte can be a biomarker.

The term “assessing” includes any form of measurement, and includes determining if an element is present or not. The terms “determining,” “measuring,” “evaluating,” “assessing,” and “assaying” are used interchangeably and include quantitative and qualitative determinations. Assessing may be relative or absolute. “Assessing the presence of” includes determining the amount of something present, and/or determining whether it is present or absent.

The term “autism spectrum disorder (ASD)” refers to a group of developmental brain disorders, having a wide range of symptoms characterized by social deficits and communication difficulties, stereotyped or repetitive behaviors and interests, and in some cases, cognitive delays. ASD is typically diagnosed according to guidelines listed in the American Psychiatric Association's Diagnostic and Statistical Manual of Mental Disorders 5th edition (DSM-5). ASDs include autism and Asperger syndrome. To various degrees, the psychiatric diagnosis of ASDs is subjective in nature as it based solely on observed behaviors and not on any quantifiable biologic, physiologic, immunologic, histologic processes or organ imaging technique.

The term “biomarker” or “marker” is a characteristic that is objectively measured and evaluated as an indicator of normal biological processes, pathological processes, or pharmacological responses to a therapeutic intervention. The biomarker can for example describe a substance whose detection indicates a particular disease state. The biomarker may be a peptide that causes disease or is associated with susceptibility to disease. In some instances, the biomarker may be a gene that causes disease or is associated with susceptibility to disease. In other instances, the biomarker is a metabolite. In any event, the biomarker can be differentially present (i.e., increased or decreased) in a biological sample from a subject or a group of subjects having a first phenotype (e.g., having a disease) as compared to a biological sample from a subject or group of subjects having a second phenotype (e.g., not having the disease). A biomarker is preferably differentially present at a level that is statistically significant (i.e., a p-value less than 0.05 and/or a q-value of less than 0.10 as determined using either Welch's T-test or Wilcoxon's rank-sum Test).

By “CENPE polypeptide” is meant a polypeptide or fragment thereof having at least about 85% amino acid identity to NCBI Accession No. NP_001804.2 and having a biological activity of a CENPE (centromere-associated protein E) polypeptide. Biological activities of a CENPE polypeptide include microtubule motor activity, microtubule binding, ATP binding, and ATPase activity. The sequence at NCBI Accession No. NP_001804.2 is shown below:

```
SEQ ID NO: 1
      1 maeegavavc vrvrplnsre eslgetaqvy wktdnnviyq vdgsksfnfd rvfhgnettk
    61 nvyeeiaapi idsaiqgyng tifaygqtas gktytmmgse dhlgviprai hdifqkikkf
  121 pdrefllrvs ymeiynetit dllcgtqkmk pliiredvnr nvyvadltee vvytsemalk
  181 witkgeksrh ygetkmnqrs srshtifrmi lesrekgeps ncegsvkvsh Inlvdlagse
  241 raaqtgaagv rlkegcninr slfilgqvik klsdgqvggf inyrdskltr ilqnslggna
  301 ktriictitp vsfdetltal qfastakymk ntpyvnevst deallkryrk eimdlkkqle
  361 evsletraqa mekdqlaqll eekdllqkvq nekienltrm lvtsssltlq qelkakrkrr
  421 vtwclgkink mknsnyadqf niptnittkt hklsinllre idesvcsesd vfsntldtls
  481 eiewnpatkl Ingeniesel nslradydnl vldyeqlrte keemelklke kndldefeal
  541 erktkkdqem qliheisnlk nlvkhaevyn qdlenelssk vellrekedq ikklqeyids
  601 qklenikmdl syslesiedp kqmkqtlfda etvaldakre saflrsenle lkekmkelat
  661 tykqmendiq lyqsqleakk kmqvdlekel qsafneitkl tslidgkvpk dllcnleleg
  721 kitdlqkeln keveenealr eevillselk slpseverlr keiqdkseel hiitsekdkl
  781 fsevvhkesr vqglleeigk tkddlattqs nykstdqefq nfktlhmdfe qkykmvleen
  841 ermnqeivnl skeaqkfdss lgalktelsy ktqelqektr evqerlneme qlkeqlenrd
  901 stlqtverek tliteklqqt leevktltqe kddlkqlqes lqierdqlks dihdtvnmni
  961 dtqeqlrnal eslkqhqeti ntlkskisee vsrnlhmeen tgetkdefqq kmvgidkkqd
1021 leakntqtlt advkdneiie qqrkifsliq eknelqqmle sviaekeqlk tdlkeniemt
1081 ienqeelrll gdelkkqqei vageknhaik kegelsrtcd rlaeveeklk eksqqlqekq
1141 qqllnvqeem semqkkinei enlknelknk eltlehmete rlelaqklne nyeevksitk
1201 erkvlkelqk sfeterdhlr gyireieatg lqtkeelkia hihlkehqet idelrrsvse
1261 ktaqiintqd lekshtklqe eipvlheege llpnvkevse tqetmnelel lteqsttkds
1321 ttlariemer lrlnekfqes qeeiksltke rdnlktikea levkhdqlke hiretlakiq
1381 esqskqeqsl nmkekdnett kivsemeqfk pkdsallrie iemlglskrl qeshdemksv
1441 akekddlqrl qevlqsesdq lkenikeiva khleteeelk vahcclkeqe etinelrvnl
1501 seketeisti qkqleaindk lqnkiqeiye keeqfnikqi sevqekvnel kqfkehrkak
1561 dsalqsiesk mleltnrlqe sqeeiqimik ekeemkrvqe alqierdqlk entkeivakm
1621 kesqekeyqf lkmtavnetq ekmceiehlk eqfetqklnl enietenirl tqilhenlee
1681 mrsvtkerdd lrsveetlkv erdqlkenlr etitrdlekq eelkivhmhl kehqetidkl
1741 rgivsektne isnmqkdleh sndalkaqdl kiqeelriah mhlkeqqeti dklrgivsek
1801 tdklsnmqkd lensnaklqe kiqelkaneh qlitlkkdvn etqkkvseme qlkkqikdqs
1861 ltlskleien Inlaqklhen leemksvmke rdnlrrveet lklerdqlke slqetkardl
1921 eiqqelktar mlskehketv dklrekisek tiqisdiqkd ldkskdelqk kiqelqkkel
1981 qllrvkedvn mshkkineme qlkkqfeaqn lsmqsvrmdn fqltkklhes leeirivake
2041 rdelrrikes lkmerdqfia tlremiardr qnhqvkpekr llsdgqqhlt eslrekcsri
2101 kellkrysem ddhyeclnrl sldlekeief qkelsmrvka nlslpylqtk hieklftanq
2161 rcsmefhrim kklkyvlsyv tkikeeqhes inkfemdfid evekqkelli kiqhlqqdcd
2221 vpsrelrdlk Inqnmdlhie eilkdfsese fpsiktefqq vlsnrkemtq fleewlntrf
2281 dieklkngiq kendricqvn nffnnriiai mnestefeer satiskeweq dlkslkekne
2341 klfknyqtlk tslasgaqvn pttqdnknph vtsratqltt ekirelensl heakesamhk
2401 eskiikmqke levtndiiak lqakvhesnk clektketiq vlqdkvalga kpykeeiedl
2461 kmklvkidle kmknakefek eisatkatve yqkevirllr enlrrsqqaq dtsvisehtd
2521 pqpsnkpltc gggsgivqnt kalilksehi rlekeisklk qqneqlikqk nellsnnqhl
2581 snevktwker tlkreahkqv tcenspkspk vtgtaskkkq itpsqckern lqdpvpkesp
2641 kscffdsrsk slpsphpvry fdnsslglcp evqnagaesv dsqpgpwhas sgkdvpeckt
2701 q
```

By “CENPE polynucleotide” is meant a polynucleotide encoding a CENPE polypeptide. An exemplary CENPE polynucleotide is provided at NCBI Accession No. NM_001813.2. The sequence at NCBI Accession No. NM_001813.2 is provided below:

| SEQ ID NO: 2 | |
|---|---|
| 1 | taaatttaaa ggcggggcgg cctgtgagcc ctgaagtgcc ggccgcggag ggtcctggcc |
| 61 | attttcctgg gaccagttca gcctgatagg atggcggagg aaggagccgt ggccgtctgc |
| 121 | gtgcgagtgc ggccgctgaa cagcagagaa gaatcacttg gagaaactgc ccaagtttac |
| 181 | tggaaaactg acaataatgt catttatcaa gttgatggaa gtaaatcctt caattttgat |
| 241 | cgtgtctttc atggtaatga aactaccaaa aatgtgtatg aagaaatagc agcaccaatc |
| 301 | atcgattctg ccatacaagg ctacaatggt actatatttg cctatggaca gactgcttca |
| 361 | ggaaaaacat ataccatgat gggttcagaa gatcatttgg gagttatacc cagggcaatt |
| 421 | catgacattt tccaaaaaat taagaagttt cctgataggg aatttctctt acgtgtatct |
| 481 | tacatggaaa tatacaatga aaccattaca gatttactct gtggcactca aaaaatgaaa |
| 541 | cctttaatta ttcgagaaga tgtcaatagg aatgtgtatg ttgctgatct cacagaagaa |
| 601 | gttgtatata catcagaaat ggctttgaaa tggattacaa agggagaaaa gagcaggcat |
| 661 | tatggagaaa caaaaatgaa tcaaagaagc agtcgttctc ataccatctt taggatgatt |
| 721 | ttggaaagca gagagaaggg tgaaccttct aattgtgaag gatctgttaa ggtatcccat |
| 781 | ttgaatttgg ttgatcttgc aggcagtgaa agagctgctc aaacaggcgc tgcaggtgtg |
| 841 | cggctcaagg aaggctgtaa tataaatcga agcttattta ttttgggaca agtgatcaag |
| 901 | aaacttagtg atggacaagt tggtggtttc ataaattatc gagatagcaa gttaacacga |
| 961 | attctccaga attccttggg aggaaatgca aagacacgta ttatctgcac aattactcca |
| 1021 | gtatcttttg atgaaacact tactgctctc cagtttgcca gtactgctaa atatatgaag |
| 1081 | aatactcctt atgttaatga ggtatcaact gatgaagctc tcctgaaaag gtatagaaaa |
| 1141 | gaaataatgg atcttaaaaa acaattagag gaggtttctt tagagacgcg ggctcaggca |
| 1201 | atggaaaaag accaattggc ccaacttttg gaagaaaaag atttgcttca gaaagtacag |
| 1261 | aatgagaaaa ttgaaaactt aacacggatg ctggtgacct cttcttccct cacgttgcaa |
| 1321 | caggaattaa aggctaaaag aaaacgaaga gttacttggt gccttggcaa aattaacaaa |
| 1381 | atgaagaact caaactatgc agatcaattt aatataccaa caaatataac aacaaaaaca |
| 1441 | cataagcttt ctataaattt attacgagaa attgatgaat ctgtctgttc agagtctgat |
| 1501 | gttttcagta acactcttga tacattaagt gagatagaat ggaatccagc aacaaagcta |
| 1561 | ctaaatcagg agaatataga aagtgagttg aactcacttc gtgctgacta tgataatctg |
| 1621 | gtattagact atgaacaact acgaacagaa aaagaagaaa tggaattgaa attaaaagaa |
| 1681 | aagaatgatt tggatgaatt tgaggctcta gaaagaaaaa ctaaaaaaga tcaagagatg |
| 1741 | caactaattc atgaaatttc gaacttaaag aatttagtta agcatgcaga agtatataat |
| 1801 | caagatcttg agaatgaact cagttcaaaa gtagagctgc ttagagaaaa ggaagaccag |
| 1861 | attaagaagc tacaggaata catagactct caaaagctag aaaatataaa aatggacttg |
| 1921 | tcatactcat tggaaagcat tgaagaccca aaacaaatga agcagactct gtttgatgct |
| 1981 | gaaactgtag cccttgatgc caagagagaa tcagcctttc ttagaagtga aaatctggag |
| 2041 | ctgaaggaga aaatgaaaga acttgcaact acatacaagc aaatggaaaa tgatattcag |
| 2101 | ttatatcaaa gccagttgga ggcaaaaaag aaaatgcaag ttgatctgga gaaagaatta |
| 2161 | caatctgctt ttaatgagat aacaaaactc acctccctta tagatggcaa agttccaaaa |
| 2221 | gatttgctct gtaatttgga attggaagga aagattactg atcttcagaa agaactaaat |
| 2281 | aaagaagttg aagaaaatga agctttgcgg gaagaagtca ttttgctttc agaattgaaa |
| 2341 | tctttacctt ctgaagtaga aaggctgagg aaagagatac aagacaaatc tgaagagctc |
| 2401 | catataataa catcagaaaa agataaattg ttttctgaag tagttcataa ggagagtaga |
| 2461 | gttcaaggtt tacttgaaga aattgggaaa acaaaagatg acctagcaac tacacagtcg |
| 2521 | aattataaaa gcactgatca agaattccaa aatttcaaaa cccttcatat ggactttgag |
| 2581 | caaaagtata agatggtcct tgaggagaat gagagaatga atcaggaaat agttaatctc |
| 2641 | tctaaagaag cccaaaaatt tgattcgagt ttgggtgctt tgaagaccga gctttcttac |
| 2701 | aagacccaag aacttcagga gaaaacacgt gaggttcaag aaagactaaa tgagatggaa |
| 2761 | cagctgaagg aacaattaga aaatagagat tctacgctgc aaactgtaga aagggagaaa |
| 2821 | acactgatta ctgagaaact gcagcaaact ttagaagaag taaaaacttt aactcaagaa |
| 2881 | aaagatgatc taaaacaact ccaagaaagc ttgcaaattg agagggacca actcaaaagt |
| 2941 | gatattcacg atactgttaa catgaatata gatactcaag aacaattacg aaatgctctt |
| 3001 | gagtctctga aacaacatca agaaacaatt aatacactaa aatcgaaaat ttctgaggaa |
| 3061 | gtttccagga atttgcatat ggaggaaaat acaggagaaa ctaaagatga atttcagcaa |
| 3121 | aagatggttg gcatagataa aaaacaggat ttggaagcta aaaataccca aacactaact |
| 3181 | gcagatgtta aggataatga gataattgag caacaaagga agatattttc tttaatacag |
| 3241 | gagaaaaatg aactccaaca aatgttagag agtgttatag cagaaaagga acaattgaag |
| 3301 | actgacctaa aggaaaatat tgaaatgacc attgaaaacc aggaagaatt aagacttctt |
| 3361 | ggggatgaac ttaaaaagca acaagagata gttgcacaag aaaagaacca tgccataaag |
| 3421 | aaagaaggag agctttctag gacctgtgac agactggcag aagttgaaga aaaactaaag |
| 3481 | gaaaagagcc agcaactcca agaaaaacag caacaacttc ttaatgtaca agaagagatg |
| 3541 | agtgagatgc agaaaaagat taatgaaata gagaatttaa agaatgaatt aaagaacaaa |
| 3601 | gaattgacat tggaacatat ggaaacagag aggcttgagt tggctcagaa acttaatgaa |
| 3661 | aattatgagg aagtgaaatc tataaccaaa gaaagaaaag ttctaaagga attacagaag |
| 3721 | tcatttgaaa cagagagaga ccaccttaga ggatatataa gagaaattga agctacaggc |
| 3781 | ctacaaacca aagaagaact aaaaattgct catattcacc taaaagaaca ccaagaaact |
| 3841 | attgatgaac taagaagaag cgtatctgag aagacagctc aaataataaa tactcaggac |
| 3901 | ttagaaaaat cccataccaa attacaagaa gagatcccag tgcttcatga ggaacaagag |
| 3961 | ttactgccta atgtgaaaga agtcagtgag actcaggaaa caatgaatga actggagtta |
| 4021 | ttaacagaac agtccacaac caaggactca acaacactgg caagaataga aatggaaagg |
| 4081 | ctcaggttga atgaaaaatt tcaagaaagt caggaagaga taaaatctct aaccaaggaa |
| 4141 | agagacaacc ttaaaacgat aaaagaagcc cttgaagtta aacatgacca gctgaaagaa |
| 4201 | catattagag aaactttggc taaaatccag gagtctcaaa gcaaacaaga acagtcctta |
| 4261 | aatatgaaag aaaaagacaa tgaaactacc aaaatcgtga gtgagatgga gcaattcaaa |
| 4321 | cccaaagatt cagcactact aaggatagaa atagaaatgc tcggattgtc caaaagactt |
| 4381 | caagaaagtc atgatgaaat gaaatctgta gctaaggaga aagatgacct acagaggctg |
| 4441 | caagaagttc ttcaatctga aagtgaccag ctcaaagaaa acataaaaga aattgtagct |
| 4501 | aaacacctgg aaactgaaga ggaacttaaa gttgctcatt gttgcctgaa agaacaagag |
| 4561 | gaaactatta atgagttaag agtgaatctt tcagagaagg aaactgaaat atcaaccatt |
| 4621 | caaaagcagt tagaagcaat caatgataaa ttacagaaca agatccaaga gatttatgag |
| 4681 | aaagaggaac aatttaatat aaaacaaatt agtgaggttc aggaaaaagt gaatgaactg |
| 4741 | aaacaattca aggagcatcg caaagccaag gattcagcac tacaaagtat agaaagtaag |
| 4801 | atgctcgagt tgaccaacag acttcaagaa agtcaagaag aaatacaaat tatgattaag |
| 4861 | gaaaaagagg aaatgaaaag agtacaggag gcccttcaga tagagagaga ccaactgaaa |
| 4921 | gaaaacacta aagaaattgt agctaaaatg aaagaatctc aagaaaaaga atatcagttt |
| 4981 | cttaagatga cagctgtcaa tgagactcag gagaaaatgt gtgaaataga acacttgaag |
| 5041 | gagcaatttg agacccagaa gttaaacctg gaaaacatag aaacggagaa tataaggttg |
| 5101 | actcagatac tacatgaaaa ccttgaagaa atgagatctg taacaaaaga aagagatgac |
| 5161 | cttaggagtg tggaggagac tctcaaagta gagagagacc agctcaagga aaaccttaga |
| 5221 | gaaactataa ctagagacct agaaaaacaa gaggagctaa aaattgttca catgcatctg |
| 5281 | aaggagcacc aagaaactat tgataaacta agagggattg tttcagagaa aacaaatgaa |
| 5341 | atatcaaata tgcaaaagga cttagaacac tcaaatgatg ccttaaaagc acaggatctg |
| 5401 | aaaatacaag aggaactaag aattgctcac atgcatctga aagagcagca ggaaactatt |
| 5461 | gacaaactca gaggaattgt ttctgagaag acagataaac tatcaaatat gcaaaaagat |
| 5521 | ttagaaaatt caaatgctaa attacaagaa aagattcaag aacttaaggc aaatgaacat |
| 5581 | caacttatta cgttaaaaaa agatgtcaat gagacacaga aaaaagtgtc tgaaatggag |
| 5641 | caactaaaga aacaaataaa agaccaaagc ttaactctga gtaaattaga aatagagaat |
| 5701 | ttaaatttgg ctcagaaact tcatgaaaac cttgaagaaa tgaaatctgt aatgaaagaa |
| 5761 | agagataatc taagaagagt agaggagaca ctcaaactgg agagagacca actcaaggaa |
| 5821 | agcctgcaag aaaccaaagc tagagatctg gaaatacaac aggaactaaa aactgctcgt |
| 5881 | atgctatcaa aagaacacaa agaaactgtt gataaactta gagaaaaaat ttcagaaaag |
| 5941 | acaattcaaa tttcagacat tcaaaaggat ttagataaat caaaagatga attacagaaa |
| 6001 | aagatccaag aacttcagaa aaaagaactt caactgctta gagtgaaaga agatgtcaat |
| 6061 | atgagtcata aaaaaattaa tgaaatggaa cagttgaaga agcaatttga ggcccaaaac |
| 6121 | ttatctatgc aaagtgtgag aatggataac ttccagttga ctaagaaact tcatgaaagc |
| 6181 | cttgaagaaa taagaattgt agctaaagaa agagatgagc taaggaggat aaaagaatct |
| 6241 | ctcaaaatgg aaagggacca attcatagca accttaaggg aaatgatagc tagagaccga |
| 6301 | cagaaccacc aagtaaaacc tgaaaaaagg ttactaagtg atggacaaca gcaccttacg |
| 6361 | gaaagcctga gagaaaagtg ctctagaata aaagagcttt tgaagagata ctcagagatg |
| 6421 | gatgatcatt atgagtgctt gaatagattg tctcttgact tggagaagga aattgaattc |
| 6481 | caaaaagagc tttcaatgag agttaaagca aacctctcac ttccctattt acaaaccaaa |
| 6541 | cacattgaaa aactttttac tgcaaaccag agatgctcca tggaattcca cagaatcatg |
| 6601 | aagaaaetga agtatgtgtt aagctatgtt acaaaaataa aagaagaaca acatgaatcc |
| 6661 | atcaataaat ttgaaatgga ttttattgat gaagtggaaa agcaaaagga attgctaatt |
| 6721 | aaaatacagc accttcaaca agattgtgat gtaccatcca gagaattaag ggatctcaaa |
| 6781 | ttgaaccaga atatggatct acatattgag gaaattctca aagatttctc agaaagtgag |
| 6841 | ttccctagca taaagactga atttcaacaa gtactaagta ataggaaaga aatgacacag |
| 6901 | tttttggaag agtggttaaa tactcgtttt gatatagaaa agcttaaaaa tggcatccag |
| 6961 | aaagaaaatg ataggatttg tcaagtgaat aacttcttta ataacagaat aattgccata |
| 7021 | atgaatgaat caacagagtt tgaggaaaga agtgctacca tatccaaaga gtgggaacag |
| 7081 | gacctgaaat cactgaaaga gaaaaatgaa aaactattta aaaactacca aacattgaag |
| 7141 | acttccttgg catctggtgc ccaggttaat cctaccacac aagacaataa gaatcctcat |
| 7201 | gttacatcaa gagctacaca gttaaccaca gagaaaattc gagagctgga aaattcactg |
| 7261 | catgaagcta aagaaagtgc tatgcataag gaaagcaaga ttataaagat gcagaaagaa |
| 7321 | cttgaggtga ctaatgacat aatagcaaaa cttcaagcca aagttcatga atcaaataaa |
| 7381 | tgccttgaaa aaacaaaaga gacaattcaa gtacttcagg acaaagttgc tttaggagct |
| 7441 | aagccatata aagaagaaat tgaagatctc aaaatgaagc ttgtgaaaat agacctagag |
| 7501 | aaaatgaaaa atgccaaaga atttgaaaag gaaatcagtg ctacaaaagc cactgtagaa |
| 7561 | tatcaaaagg aagttataag gctattgaga gaaaatctca gaagaagtca acaggcccaa |
| 7621 | gatacctcag tgatatcaga acatactgat cctcagcctt caaataaacc cttaacttgt |
| 7681 | ggaggtggca gcggcattgt acaaaacaca aaagctctta ttttgaaaag tgaacatata |
| 7741 | aggctagaaa aagaaatttc taagttaaag cagcaaaatg aacagctaat aaaacaaaag |
| 7801 | aatgaattgt taagcaataa tcagcatctt tccaatgagg tcaaaacttg gaaggaaaga |
| 7861 | acccttaaaa gagaggctca caaacaagta acttgtgaga attctccaaa gtctcctaaa |
| 7921 | gtgactggaa cagcttctaa aaagaaacaa attacaccct ctcaatgcaa ggaacggaat |
| 7981 | ttacaagatc ctgtgccaaa ggaatcacca aaatcttgtt tttttgatag ccgatcaaag |
| 8041 | tctttaccat cacctcatcc agttcgctat tttgataact caagtttagg cctttgtcca |
| 8101 | gaggtgcaaa atgcaggagc agagagtgtg gattctcagc caggtccttg gcacgcctcc |
| 8161 | tcaggcaagg atgtgcctga gtgcaaaact cagtagactc ctctttgtca cttctctgga |
| 8221 | gatccagcat tccttatttg gaaatgactt tgtttatgtg tctatccctg gtaatgatgt |
| 8281 | tgtagtgcag cttaatttca attcagtctt tactttgcca ctagagttga aagataaggg |
| 8341 | aacaggaaat gaatgcattg tggtaattta gaatggtgat agcaatacct tcttcttgca |
| 8401 | tatggtaata cttttaaaag ttgaattgtt ttatttattt gtatattttg taaagaataa |
| 8461 | agttattgaa agaaatgtaa agttatctac atgacttagc atattccaaa gcataataca |
| 8521 | tacattaata taaaacatca ttttattaac aaaattgtaa atgtttttaa taccttacac |
| 8581 | attcaataaa tgtttagtag ttctgaatca ccaaaaaaaa aaaaaaaaaa |

By “CYP2S1 polypeptide” is meant a polypeptide or fragment thereof having at least about 85% amino acid identity to NCBI Accession No. NP_085125.1 and having a biological activity of a CYP2S1 (cytochrome P450, family 2) polypeptide. Biological activities of a CYP2S1 polypeptide include steroid hydroxylase activity, oxidoreductase activity, and extrahepatic xenobiotic metabolism. The sequence at NCBI Accession No. NP_085125.1 is shown below:

```
SEQ ID NO: 3
    1 meatgtwall lalalllllt lalsgtrarg
        hlppgptplp llgnllqlrp galysglmrl
  61 skkygpvfti ylgpwrpvvv ivgqeavrea
        lggqaeefsg rgtvamlegt fdghgvffsn
121 gerwrqlrkf tmlalrdlgm gkregeeliq
        aearclvetf qgtegrpfdp slllaqatsn
181 vvcsllfglr fsyedkefqa vvraaggtll
        gvssqggqty emfswflrpl pgphkqllhh
241 vstlaaftvr qvqqhqgnld asgpardlvd
        afllkmaqee qnpgteftnk nmlmtviyll
301 fagtmtvstt vgytllllmk yphvqkwvre
        elnrelgagq apslgdrtrl pytdavlhea
361 qrllalvpmg iprtlmrttr frgytlpqgt
        evfpllgsil hdpnifkhpe efnpdrflda
421 dgrfrkheaf lpfslgkrvc lgeglakael
        flffttilqa fslespcppd tlslkptvsg
481 lfnippafql qvrptdlhst tqtr
```

By “CYP2S1 polynucleotide” is meant a polynucleotide encoding a CYP2S1 polypeptide. An exemplary CYP2S1 polynucleotide sequence is provided at NCBI Accession No. NM_030622.7. The exemplary sequence provided at NCBI Accession No. NM_030622.7 is reproduced below:

| SEQ ID NO: 4 | |
|---|---|
| 1 | cctaactagc ccagccgcgc ggagcgcctg ggagaggaga aggagccgac ctgccgagat |
| 61 | ggaggcgacc ggcacctggg cgctgctgct ggcgctggcg ctgctcctgc tgctgacgct |
| 121 | ggcgctgtcc gggaccaggg cccgaggcca cctgcccccc gggcccacgc cgctaccact |
| 181 | gctgggaaac ctcctgcagc tacggcccgg ggcgctgtat tcagggctca tgcggctgag |
| 241 | taagaagtac ggaccggtgt tcaccatcta cctgggaccc tggcggcctg tggtggtcct |
| 301 | ggttgggcag gaggctgtgc gggaggccct gggaggtcag gctgaggagt tcagcggccg |
| 361 | gggaaccgta gcgatgctgg aagggacttt tgatggccat ggggttttct tctccaacgg |
| 421 | ggagcggtgg aggcagctga ggaagtttac catgcttgct ctgcgggacc tgggcatggg |
| 481 | gaagcgagaa ggcgaggagc tgatccaggc ggaggcccgg tgtctggtgg agacattcca |
| 541 | ggggacagaa ggacgcccat tcgatccctc cctgctgctg gcccaggcca cctccaacgt |
| 601 | agtctgctcc ctcctctttg gcctccgctt ctcctatgag gataaggagt tccaggccgt |
| 661 | ggtccgggca gctggtggta ccctgctggg agtcagctcc caggggggtc agacctacga |
| 721 | gatgttctcc tggttcctgc ggcccctgcc aggcccccac aagcagctcc tccaccacgt |
| 781 | cagcaccttg gctgccttca cagtccggca ggtgcagcag caccagggga acctggatgc |
| 841 | ttcgggcccc gcacgtgacc ttgtcgatgc cttcctgctg aagatggcac aggaggaaca |
| 901 | aaacccaggc acagaattca ccaacaagaa catgctgatg acagtcattt atttgctgtt |
| 961 | tgctgggacg atgacggtca gcaccacggt cggctatacc ctcctgctcc tgatgaaata |
| 1021 | ccctcatgtc caaaagtggg tacgtgagga gctgaatcgg gagctggggg ctggccaggc |
| 1081 | accaagccta ggggaccgta cccgcctccc ttacaccgac gcggttctgc atgaggcgca |
| 1141 | gcggctgctg gcgctggtgc ccatgggaat accccgcacc ctcatgcgga ccacccgctt |
| 1201 | ccgagggtac accctgcccc agggcacgga ggtcttcccc ctccttggct ccatcctgca |
| 1261 | tgaccccaac atcttcaagc acccagaaga gttcaaccca gaccgtttcc tggatgcaga |
| 1321 | tggacggttc aggaagcatg aggcgttcct gcccttctcc ttagggaagc gtgtctgcct |
| 1381 | tggagagggc ctggcaaaag cggagctctt cctcttcttc accaccatcc tacaagcctt |
| 1441 | ctccctggag agcccgtgcc cgccggacac cctgagcctc aagcccaccg tcagtggcct |
| 1501 | tttcaacatt cccccagcct tccagctgca agtccgtccc actgaccttc actccaccac |
| 1561 | gcagaccaga tgaaggaagg caacttggaa gtggtgggtg cccaggacgg tgcctccagc |
| 1621 | ctcaacagtg ggcatggaca gggttaatgt ctccagagtg tacactgcag gcagccacat |
| 1681 | ttacacgcct gcagttgttt tccggagtct gtcccacggc ccacacgctc acttgactca |
| 1741 | tgctgctaag atgcacaacc gcacacccat acacaactac aagggccaca aagcaactgc |
| 1801 | tgggttagct ttccacagac ataaatatag tccatctgca atcacaagca catagccagg |
| 1861 | taacccacca actcccctgg atctgcagcc cacacgtggg agtctggctg tcaccttcac |
| 1921 | aagccacaga aacggccaca catgttcaca gctcacacgc cctctccatt catcgaactt |
| 1981 | ctcagtgtcc ctgtccctgg tgcctggcac agggaacagc atgccccctc cggggtcatg |
| 2041 | ccacccagag actgtcgctg tctatggccc caactcatgc tccctctctt ggctacacca |
| 2101 | ctctcccagc ctgtgaccac cgatgtccac acacccccaa ccacttgtcc acacagctac |
| 2161 | ccacgtacga catcgtcctg gctccccaga gtatcttccc actgagacac gccgccccca |
| 2221 | cagaggcaca gtccccagcc acctctgcaa ctgcagccct cagtcacccc tttttaagca |
| 2281 | ccctgattct accaaatgca aacacatctg ggtctgcgat tatgcacaga gactttggac |
| 2341 | atacgaggac cctcagaccg gaggaacacc tgcccaaccc caacacgtgc ttatgtaacc |
| 2401 | acgtggaaag cggcccctgc tgcccctcca cacacacata cacactcact gatctacagc |
| 2461 | ccctgttcgg cgtcagagtc cccactagac ccagtggaag gggttagaga ccaagtaggg |
| 2521 | gccagtttcc aattcaccct gtcagggagt gagccggatc tgacgttcct tgtgacttaa |
| 2581 | gggtccggct tgggaattaa agtttgtttc tggcctttag cctaaaaaaa aaaaaaaaaa |
| 2641 | aaaaaaaaaa aaaaaaaaaa |

By “capture reagent” is meant a reagent that specifically binds a nucleic acid molecule or polypeptide to select or isolate the nucleic acid molecule or polypeptide.

By “decreases” is meant a negative alteration of at least 10%, 25%, 50%, 75%, 100%, 200%, 300%, 400%, 500%, 1000%, or more.

By “detect” refers to identifying the presence, absence, level, or concentration of an agent.

By “detectable” is meant a moiety that when linked to a molecule of interest renders the latter detectable. Such detection may be via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health. Preferably, the animal is a mammal. More preferably, the mammal is a human.

A disease or disorder is “alleviated” if the severity of a sign or symptom of the disease or disorder, the frequency with which such a sign or symptom is experienced by a patient, or both, is reduced.

An “effective amount” or “therapeutically effective amount” of a compound is that amount of compound which is sufficient to provide a beneficial effect to the subject to which the compound is administered. An “effective amount” of a delivery vehicle is that amount sufficient to effectively bind or deliver a compound.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

The term “expression” as used herein is defined as the transcription and/or translation of a particular nucleotide sequence driven by its promoter.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600,700, 800, 900, or 1000 nucleotides or amino acids.

By “FCER1A polypeptide” is meant a polypeptide or fragment thereof having at least about 85% amino acid identity to NCBI Accession No. NP_001992.1 and having a biological activity of a FCER1A (high affinity IgE receptor FcεR1) polypeptide. Biological activities of a FCER1A polypeptide include IgE receptor activity and IgE binding. The sequence at NCBI Accession No. NP_001992.1 is shown below:

```
SEQ ID NO: 5
    1 mapamesptl lcvallffap dgvlavpqkp
        kvslnppwnr ifkgenvtlt cngnnffevs
  61 stkwfhngsl seetnsslni vnakfedsge
        ykcqhqqvne sepvylevfs dwlllqasae
121 vvmegqplfl rchgwrnwdv ykviyykdge
        alkywyenhn isitnatved sgtyyctgkv
181 wqldyesepl nitvikapre kywlqffipl
        lvvilfavdt glfistqqqv tfllkikrtr
241 kgfrllnphp kpnpknn
```

By “FCER1A polynucleotide” is meant a polynucleotide encoding a FCER1A polypeptide. An exemplary PCER1A polynucleotide sequence is provided at NCBI Accession No. NM_002001.3. The exemplary sequence provided at NCBI Accession No. NM_002001.3 is reproduced below:

| SEQ ID NO: 6 | |
|---|---|
| 1 | tactaagagt ctccagcatc ctccacctgt ctaccaccga gcatgggcct atatttgaag |
| 61 | ccttagatct ctccagcaca gtaagcacca ggagtccatg aagaagatgg ctcctgccat |
| 121 | ggaatcccct actctactgt gtgtagcctt actgttcttc gctccagatg gcgtgttagc |
| 181 | agtccctcag aaacctaagg tctccttgaa ccctccatgg aatagaatat ttaaaggaga |
| 241 | gaatgtgact cttacatgta atgggaacaa tttctttgaa gtcagttcca ccaaatggtt |
| 301 | ccacaatggc agcctttcag aagagacaaa ttcaagtttg aatattgtga atgccaaatt |
| 361 | tgaagacagt ggagaataca aatgtcagca ccaacaagtt aatgagagtg aacctgtgta |
| 421 | cctggaagtc ttcagtgact ggctgctcct tcaggcctct gctgaggtgg tgatggaggg |
| 481 | ccagcccctc ttcctcaggt gccatggttg gaggaactgg gatgtgtaca aggtgatcta |
| 541 | ttataaggat ggtgaagctc tcaagtactg gtatgagaac cacaacatct ccattacaaa |
| 601 | tgccacagtt gaagacagtg gaacctacta ctgtacgggc aaagtgtggc agctggacta |
| 661 | tgagtctgag cccctcaaca ttactgtaat aaaagctccg cgtgagaagt actggctaca |
| 721 | attttttatc ccattgttgg tggtgattct gtttgctgtg gacacaggat tatttatctc |
| 781 | aactcagcag caggtcacat ttctcttgaa gattaagaga accaggaaag gcttcagact |
| 841 | tctgaaccca catcctaagc caaaccccaa aaacaactga tataattact caagaaatat |
| 901 | ttgcaacatt agtttttttc cagcatcagc aattgctact caattgtcaa acacagcttg |
| 961 | caatatacat agaaacgtct gtgctcaagg atttatagaa atgcttcatt aaactgagtg |
| 1021 | aaactggtta agtggcatgt aatagtaagt gctcaattaa cattggttga ataaatgaga |
| 1081 | gaatgaatag attcatttat tagcatttgt aaaagagatg ttcaatttca ataaaataaa |
| 1141 | tataaaacca tgtaacagaa tgcttctgag taaaaaaaaa aaaaaaaaaa aaaaaaaa |

By “genotype” is meant the genetic composition of a cell, organism, or individual.

By “IL1RN polypeptide” is meant a polypeptide or fragment thereof having at least about 85% amino acid identity to NCBI Accession No. NP_776214.1 and having a biological activity of a IL1RN (interleukin 1 receptor antagonist) polypeptide. Biological activities of a IL1RN polypeptide include cytokine activity, interleukin-1 receptor binding, and interleukin-1 receptor antagonist activity. The sequence at NCBI Accession No. NP_776214.1 is shown below:

```
SEQ ID NO: 7
    1 meicrglrsh litlllflfh seticrpsgr
        ksskmqafri wdvnqktfyl rnnqlvagyl
  61 qgpnvnleek idvvpiepha lflgihggkm
        clscvksgde trlqleavni tdlsenrkqd
121 krfafirsds gpttsfesaa cpgwflctam
        eadqpvsltn mpdegvmvtk fyfqede
```

By “IL1RN polynucleotide” is meant a polynucleotide encoding a IL1RN polypeptide. An exemplary IL1RN polynucleotide sequence is provided at NCBI Accession No. NM_173842.2. The exemplary sequence provided at NCBI Accession No. NM_173842.2 is reproduced below:

| SEQ ID NO: 8 | |
|---|---|
| 1 | atttctttat aaaccacaac tctgggcccg caatggcagt ccactgcctt gctgcagtca |
| 61 | cagaatggaa atctgcagag gcctccgcag tcacctaatc actctcctcc tcttcctgtt |
| 121 | ccattcagag acgatctgcc gaccctctgg gagaaaatcc agcaagatgc aagccttcag |
| 181 | aatctgggat gttaaccaga agaccttcta tctgaggaac aaccaactag ttgctggata |
| 241 | cttgcaagga ccaaatgtca atttagaaga aaagatagat gtggtaccca ttgagcctca |
| 301 | tgctctgttc ttgggaatcc atggagggaa gatgtgcctg tcctgtgtca agtctggtga |
| 361 | tgagaccaga ctccagctgg aggcagttaa catcactgac ctgagcgaga acagaaagca |
| 421 | ggacaagcgc ttcgccttca tccgctcaga cagtggcccc accaccagtt ttgagtctgc |
| 481 | cgcctgcccc ggttggttcc tctgcacagc gatggaagct gaccagcccg tcagcctcac |
| 541 | caatatgcct gacgaaggcg tcatggtcac caaattctac ttccaggagg acgagtagta |
| 601 | ctgcccaggc ctgcctgttc ccattcttgc atggcaagga ctgcagggac tgccagtccc |
| 661 | cctgccccag ggctcccggc tatgggggca ctgaggacca gccattgagg ggtggaccct |
| 721 | cagaaggcgt cacaacaacc tggtcacagg actctgcctc ctcttcaact gaccagcctc |
| 781 | catgctgcct ccagaatggt ctttctaatg tgtgaatcag agcacagcag cccctgcaca |
| 841 | aagcccttcc atgtcgcctc tgcattcagg atcaaacccc gaccacctgc ccaacctgct |
| 901 | ctcctcttgc cactgcctct tcctccctca ttccaccttc ccatgccctg gatccatcag |
| 961 | gccacttgat gacccccaac caagtggctc ccacaccctg ttttacaaaa aagaaaagac |
| 1021 | cagtccatga gggaggtttt taagggtttg tggaaaatga aaattaggat ttcatgattt |
| 1081 | ttttttttca gtccccgtga aggagagccc ttcatttgga gattatgttc tttcggggag |
| 1141 | aggctgagga cttaaaatat tcctgcattt gtgaaatgat ggtgaaagta agtggtagct |
| 1201 | tttcccttct ttttcttctt tttttgtgat gtcccaactt gtaaaaatta aaagttatgg |
| 1261 | tactatgtta gccccataat tttttttttc cttttaaaac acttccataa tctggactcc |
| 1321 | tctgtccagg cactgctgcc cagcctccaa gctccatctc cactccagat tttttacagc |
| 1381 | tgcctgcagt actttacctc ctatcagaag tttctcagct cccaaggctc tgagcaaatg |
| 1441 | tggctcctgg gggttctttc ttcctctgct gaaggaataa attgctcctt gacattgtag |
| 1501 | agcttctggc acttggagac ttgtatgaaa gatggctgtg cctctgcctg tctcccccac |
| 1561 | cgggctggga gctctgcaga gcaggaaaca tgactcgtat atgtctcagg tccctgcagg |
| 1621 | gccaagcacc tagcctcgct cttggcaggt actcagcgaa tgaatgctgt atatgttggg |
| 1681 | tgcaaagttc cctacttcct gtgacttcag ctctgtttta caataaaatc ttgaaaatgc |
| 1741 | ctaaaaaaaa aaaaaaaaaa aaaaaaaaaa aaaaaaaaaa aaaaaaaaaa aaaa |

By “increases” is meant a positive alteration of at least 10%, 25%, 50%, 75%, 100%, 200%, 300%, 400%, 500%, 1000%, or more.

As used herein, an “instructional material” includes a publication, a recording, a diagram, or any other medium of expression which can be used to communicate the usefulness of the compositions and methods of the invention. The instructional material of the kit of the invention may, for example, be affixed to a container which contains the nucleic acid, peptide, and/or composition of the invention or be shipped together with a container which contains the nucleic acid, peptide, and/or composition. Alternatively, the instructional material may be shipped separately from the container with the intention that the instructional material and the compound be used cooperatively by the recipient.

By “marker profile” is meant a characterization of the expression or expression level of two or more polypeptides or polynucleotides

“Measuring” or “measurement,” or alternatively “detecting” or “detection,” means assessing the presence, absence, quantity or amount (which can be an effective amount) of either a given substance within a clinical or subject-derived sample, including the derivation of qualitative or quantitative concentration levels of such substances, or otherwise evaluating the values or categorization of a subject's clinical parameters.

By “MTHFD2 polypeptide” is meant a polypeptide or fragment thereof having at least about 85% amino acid identity to NCBI Accession No. NP_006627.2 and having a biological activity of a MTHFD2 (methylenetetrahydrofolate dehydrogenase (NADP+ dependent) 2, methenyltetrahydrofolate cyclohydrolase) polypeptide. Biological activities of a MTHFD2 polypeptide include methylenetetrahydrofolate dehydrogenase activity and methenyltetrahydrofolate cyclohydrolase activity. The sequence at NCBI Accession No. NP_006627.2 is shown below:

| SEQ ID NO: 9 | |
|---|---|
| 1 | maatslmsal aarllqpahs cslrlrpfhl aavrneavvi sgrklaqqik qevrqeveew |
| 61 | vasgnkrphl svilvgenpa shsyvlnktr aaavvginse timkpasise eellnlinkl |
| 121 | nnddnvdgll vqlplpehid erricnavsp dkdvdgfhvi nvgrmcldqy smlpatpwgv |
| 181 | weiikrtgip tlgknvvvag rsknvgmpia mllhtdgahe rpggdatvti shrytpkeql |
| 241 | kkhtiladiv isaagipnli tadmikegaa vidvginrvh dpvtakpklv gdvdfegvrq |
| 301 | kagyitpvpg gvgpmtvaml mkntiiaakk vlrleerevl kskelgvatn |

By “MTHPD2 polynucleotide” is meant a polynucleotide encoding an MTHPD2 polypeptide. An exemplary MTHPD2 polynucleotide is provided at NCBI Accession No. NM_006636.3. The sequence at NCBI Accession No. NM_006636.3 is provided below:

| SEQ ID NO: 10 | |
|---|---|
| 1 | ggggcctgcc acgaggccgc agtataaccg cgtggcccgc gcgcgcgctt ccctcccggc |
| 61 | gcagtcaccg gcgcggtcta tggctgcgac ttctctaatg tctgctttgg ctgcccggct |
| 121 | gctgcagccc gcgcacagct gctcccttcg ccttcgccct ttccacctcg cggcagttcg |
| 181 | aaatgaagct gttgtcattt ctggaaggaa actggcccag cagatcaagc aggaagtgcg |
| 241 | gcaggaggta gaagagtggg tggcctcagg caacaaacgg ccacacctga gtgtgatcct |
| 301 | ggttggcgag aatcctgcaa gtcactccta tgtcctcaac aaaaccaggg cagctgcagt |
| 361 | tgtgggaatc aacagtgaga caattatgaa accagcttca atttcagagg aagaattgtt |
| 421 | gaatttaatc aataaactga ataatgatga taatgtagat ggcctccttg ttcagttgcc |
| 481 | tcttccagag catattgatg agagaaggat ctgcaatgct gtttctccag acaaggatgt |
| 541 | tgatggcttt catgtaatta atgtaggacg aatgtgtttg gatcagtatt ccatgttacc |
| 601 | ggctactcca tggggtgtgt gggaaataat caagcgaact ggcattccaa ccctagggaa |
| 661 | gaatgtggtt gtggctggaa ggtcaaaaaa cgttggaatg cccattgcaa tgttactgca |
| 721 | cacagatggg gcgcatgaac gtcccggagg tgatgccact gttacaatat ctcatcgata |
| 781 | tactcccaaa gagcagttga agaaacatac aattcttgca gatattgtaa tatctgctgc |
| 841 | aggtattcca aatctgatca cagcagatat gatcaaggaa ggagcagcag tcattgatgt |
| 901 | gggaataaat agagttcacg atcctgtaac tgccaaaccc aagttggttg gagatgtgga |
| 961 | ttttgaagga gtcagacaaa aagctgggta tatcactcca gttcctggag gtgttggccc |
| 1021 | catgacagtg gcaatgctaa tgaagaatac cattattgct gcaaaaaagg tgctgaggct |
| 1081 | tgaagagcga gaagtgctga agtctaaaga gcttggggta gccactaatt aactactgtg |
| 1141 | tcttctgtgt cacaaacagc actccaggcc agctcaagaa gcaaagcagg ccaatagaaa |
| 1201 | tgcaatattt ttaatttatt ctactgaaat ggtttaaaat gatgccttgt atttattgaa |
| 1261 | agcttaaatg ggtgggtgtt tctgcacata cctctgcagt acctcaccag ggagcattcc |
| 1321 | agtatcatgc agggtcctgt gatctagcca ggagcagcca ttaacctagt gattaatatg |
| 1381 | ggagacatta ccatatggag gatggatgct tcactttgtc aagcacctca gttacacatt |
| 1441 | cgccttttct aggattgcat ttcccaagtg ctattgcaat aacagttgat actcatttta |
| 1501 | ggtaccaaac cttttgagtt caactgatca aaccaaagga aaagtgttgc tagagaaaat |
| 1561 | tagggaaaag gtgaaaaaga aaaaatggta gtaattgagc agaaaaaaat taatttatat |
| 1621 | atgtattgat tggcaaccag atttatctaa gtagaactga attggctagg aaaaaagaaa |
| 1681 | aactgcatgt taatcatttt cctaagctgt ccttttgagg cttagtcagt ttattgggaa |
| 1741 | aatgtttagg attattcctt gctattagta ctcattttat gtatgttacc cttcagtaag |
| 1801 | ttctccccat tttagttttc taggactgaa aggattcttt tctacattat acatgtgtgt |
| 1861 | tgtcatattt ggcttttgct atatacttta acttcattgt taaatttttg tattgtatag |
| 1921 | tttctttggt gtatcttaaa acctattttt gaaaaacaaa cttggcttga taatcatttg |
| 1981 | ggcagcttgg gtaagtacgc aacttacttt tccaccaaag aactgtcagc agctgcctgc |
| 2041 | ttttctgtga tgtatgtatc ctgttgactt ttccagaaat tttttaagag tttgagttac |
| 2101 | tattgaattt aatcagactt tctgattaaa gggttttctt tcttttttaa taaaacacat |
| 2161 | ctgtctggta tggtatgaat ttctgaaaaa aaaaaaaaaa aaaaaaaa |

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

As used herein a “nucleic acid or oligonucleotide probe” is defined as a nucleic acid capable of binding to a target nucleic acid of complementary sequence through one or more types of chemical bonds, usually through complementary base pairing, usually through hydrogen bond formation. As used herein, a probe may include natural (i.e., A, G, C, or T) or modified bases (7-deazaguanosine, inosine, etc.). In addition, the bases in a probe may be joined by a linkage other than a phosphodiester bond, so long as it does not interfere with hybridization. It will be understood by one of skill in the art that probes may bind target sequences lacking complete complementarity with the probe sequence depending upon the stringency of the hybridization conditions. The probes are preferably directly labeled with isotopes, for example, chromophores, lumiphores, chromogens, or indirectly labeled with biotin to which a streptavidin complex may later bind. By assaying for the presence or absence of the probe, one can detect the presence or absence of a target gene of interest.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

As used herein, “quantitative trait” refer to a phenotype or characteristics of an individual that can be attributed to the effect two or more genes.

As used herein, “quantitative trait locus (QTL)” refers to a DNA sequence or segment located within the genome containing or linked to the genes that underlie a quantitative trait.

As used herein, “expression quantitative trait loci (eQTLs)” are genomic loci that regulate expression levels of mRNAs or proteins. The abundance of a gene transcript is directly modified by polymorphisms in regulatory elements that alter the level of a gene transcript. These can be mapped and the level of a gene transcript can be used as a quantitative trait. Mapping eQTLs is performed using standard QTL mapping methods that test the linkage between variation in expression and genetic polymorphisms. In one embodiment, eQTL is determined by statistical regression of the genotype of an SNP and the expression for the transcript.

By “reference” is meant a standard or control condition. In one embodiment, the level of gene expression in a tissue sample of a subject having GI Symptomatic ASD is compared to the gene expression in a tissue sample from a control subject.

“Sample” or “biological sample” as used herein means a biological material isolated from a subject, including any tissue, cell, fluid, or other material obtained or derived from the subject (e.g., a human). The biological sample may contain any biological material suitable for detecting the desired analytes, and may comprise cellular and/or non-cellular material obtained from the subject. In various embodiments, the biological sample may be obtained from the small bowel, stomach, or esophagus. In particular embodiments, the biological sample is a blood sample.

By “single nucleotide polymorphism” or “SNP” is meant a DNA sequence variation occurring when a single nucleotide in the genome differs between members of a biological species or paired chromosomes in an individual. SNPs are used as genetic markers for variant alleles.

By “SIGLEC17P polypeptide” is meant a polypeptide or fragment thereof having at least about 85% amino acid identity to GenBank No. AAF28901.1 and having a biological activity of a SIGLEC17P (sialic acid binding Ig-like lectin 17) polypeptide. SIGLEC17P polypeptide is also referred to herein as “SIGLECP3 polypeptide”. Biological activities of a SIGLEC17P polypeptide include sialic acid binding. The sequence at GenBank No. AAF28901.1 is shown below:

| SEQ ID NO: 11 | |
|---|---|
| 1 | erccrcccrc pccgqgprsg ckipagdarv rdgaggsvhl calfgllprv wlerfypclw |
| 61 | plvpgkgqcr pgdsmatnns tqkvqketqg rfhllgdpsr nncslsirda rrrdngsyff |
| 121 | wvarrrtkfs ykysplsvyv talthrpdil ipeflksghp snltcsvpwv ceqgtppifs |
| 181 | wmsaaptslg prtlhsselt iiprpqdhgt nlicqvtfpg agvttertiq lsvswksgtv |
| 241 | eevvvlavgv vavkilllcl cliilsfhkk kavravevee nvyavmg |

By “SIGLEC17P polynucleotide” or “SIGLECP3 polynucleotide” is meant a polynucleotide encoding a SIGLEC17P polypeptide. An exemplary SIGLEC17P polynucleotide is provided at NCBI Accession No. NR_002804. The sequence at NCBI Accession No. NR_002804 is provided below:

SEQ ID NO: 12
CCAAGATCTCATGCTCCTCCCCACAGCCCTCTTCTCTGCTCACACAGGA
AGCCCAGGAAGCCTCTGCCTCAGAGATGCTGCCGCTGCTGCTGCCGCTG
CCCCTGCTGTGGGCAGGGCCCTCGCTCAGGATGCAAGATTCCGGCTGGA
GATGCCAGAGTCCGTGACGGTGCAGGAGGGTCTGTGCATCTTTGTGCAC
TGTTCGGTCTTCTACCTCGAGTATGGCTGGAAAGATTCTACCCCTGCTT
ATGGCCACTGGTTCCGGGAAGGGGTCAGTGTAGACCAGGAGACTCCAGT
GGCCACAAACAACTCAACTCAAAAAGTGCAGAAGGAGACCCAGGGCCGA
TTCCACCTCCTCGGTGATCCCTCAAGGAACAACTGCTCCCTGAGCATCA
GAGACGCCAGGAGGAGGGACAACGGTTCATACTTCTTTTGGGTGGCGAG
AGGAAGAACAAAATTTAGTTACAAATATTCCCCGCTCTCTGTGTATGTG
ACAGCCCTGACCCACAGGCCCGACATCCTCATCCCGGAGTTCCTAAAGT
CTGGCCATCCCAGCAACCTGACCTGCTCTGTGCCCTGGGTCTGTGAGCA
GGGAACACCCCCCATCTTCTCCTGGATGTCAGCTGCCCCCACCTCCCTG
GGCCCCAGGACCCTCCACTCCTCAGTGCTCACGATCATCCCACGGCCTC
AGGACCACGGCACCAACCTCATCTGTCAGGTGACGTTCCCCGGAGCTGG
TGTGACCACGGAGAGAACCATCCAGCTCAGTGTCTCCTGGAAATCAGGA
ACCGTGGAAGAGGTGGTTGTTTTGGCCGTGGGGGTAGTGGCTGTGAAGA
TCCTGCTTCTCTGCCTTTGCCTCATCATCCTCAGGTAAGCACTGCCTGA
AGACCAAGGACAGGCATGGGGAGGGCAGAGGACATGATGCTGAATCCCA
GAATCTCAATCCTGGGGGTATGCAGACAGTTTACGTGGTCCTGGGGCCA
GGCTGGAGGCTGAATTGGTGGTGAGAATTACACATGGGCCATTTGTGGT
CAGTTTGTGTCTGTCCCAACTGAGGGCCAAATGCCAGGATGGGGAGCTT
CCTGTTGTCATCAAGGAAGTCTAGACCTGCTCTTCCTCCCTGTGATCCC
TCCAGCCTCTAGCAGGGCACAGGAAGTTGAGTTGGCTGCCCTTTGCTCC
CTTCATGTGGCCACACTTACAGGTCCTTGTCTCTTCACTCAATGTCAGT
TTCCACAAGAAGAAGGCGGTGAGGGCAGTGGAGGTTGAGGAGAATGTAT
ATGCTGTCATGGGTTAATCTCTCAGGTGAGTGATGTGGGCCTCTCACTC
TTCAACATCCTGCTGGATAACTCCTCCACAATGGCCTCCAGGATTGCTC
TGCCCATCATGGCCAAAGTTAAGCCAACTGTCTTCCTCCTCAAACCTAC
TTTTCCTGGGATGTGGGTTCTTCATCCTGCAGATGACAAAGAGGCCTCA
TCTCTAAAGTCAGAACCTGGGTGTGGGTCTCCATCTTGACCCCCCTCCC
TTCTCTAGATCCCATAAATTACTAGCTCTTGTCCCTCCTTCTCCTAAGC
AGGGCTCATCTTGATGCCCTTTTCTCCATCCTGACCCTGGTCATTCTTG
CGGCCTCACCTCTTCCCTGATCACTGAACCCTTTTCACCTCCTGCCTCC
ATCTCTCCCCAACACAGGCCTCCAGACTGTACTTCCAGATGTCTCCTCA
TCCAGTTCCTCCACAGTCTGAATGGCCATGTTTCCTCTTCATTGCTGGA
GAATGAAGTGCAAATGCCACTGCCTGGACTGAAGGCCTTTCACGATCTG
TCTTCTGCTGGACTCTGCTCCTGATCCCCCTTCTCCTTGCATCACCCGA
AGTCTCCCTACACCCACCAGGCCAAGCCCTCTGTGATTCTGAGACTTTG
CATGTGTAGTTACTTCTCCTGAAATGGCCTTCCTCCCCATTCCTGCCAA
TCCAGGTCCTTATCATCCTTCAGGTTGTCTTAAATGTCATCCAGGTGTG
TGTATTTTTATGTAATCCTTGTATGATATTAAGCGGAGATGTGGCATTT
GTTCATTAATTTGTAGACATATTCAGTAACCATACTGAATACATATAAT
GACTATGTGCCAGCATTTCCGTATGTGCAGAAGTTCATCAATAGATATA
GACTCAAAGAGCTCTGTCATCAAGCTGTTGTTCTGAAGAGCAGAAGGAT
ACAAATAAAAAGAAATAAGTAAAATAAAAAAAAAAAAAAAAAAAAA

By “specifically binds” is meant a compound or antibody that recognizes and binds a polypeptide of the invention, but which does not substantially recognize and bind other molecules in a sample. By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and most preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e″³ and e″¹⁰⁰ indicating a closely related sequence.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. C in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42° C. in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS.

Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “target nucleic acid molecule” is meant a nucleic acid or biomarker of the sample that is to be detected.

By “TNFAIP3 polypeptide” is meant a polypeptide or fragment thereof having at least about 85% amino acid identity to NCBI Accession No. NP_006281.1 and having a biological activity of a TNFAIP3 (tumor necrosis factor, alpha-induced protein 3) polypeptide. Biological activities of a TNFAIP3 polypeptide include ubiquitin ligase activity and deubiquitinase activity. The sequence at NCBI Accession No. NP_006281.1 is shown below:

| SEQ ID NO: 13 | |
|---|---|
| 1 | maeqvlpqal ylsnmrkavk irertpedif kptngiihhf ktmhrytlem frtcqfcpqf |
| 61 | reiihkalid rniqatlesq kklnwcrevr klvalktngd gnclmhatsq ymwgvqdtdl |
| 121 | vlrkalfstl ketdtrnfkf rwqleslksq efvetglcyd trnwndewdn likmastdtp |
| 181 | marsglqyns leeihifvlc nilrrpiivi sdkmlrsles gsnfaplkvg giylplhwpa |
| 241 | qecyrypivl gydshhfvpl vtlkdsgpei ravplvnrdr grfedlkvhf ltdpenemke |
| 301 | kllkeylmvi eipvqgwdhg tthlinaakl deanlpkein lvddyfelvq heykkwqens |
| 361 | eqgrreghaq npmepsvpql slmdvkcetp ncpffmsvnt qplchecser rqknqnklpk |
| 421 | lnskpgpegl pgmalgasrg eayeplawnp eestggphsa pptapspflf settamkcrs |
| 481 | pgcpftlnvq hngfcerchn arqlhashap dhtrhldpgk cqaclqdvtr tfngicstcf |
| 541 | krttaeasss lstslppsch qrsksdpsrl vrspsphsch ragndapagc lsqaartpgd |
| 601 | rtgtskcrka gcvyfgtpen kgfctlcfie yrenkhfaaa sgkvsptasr fqntipclgr |
| 661 | ecgtlgstmf egycqkcfie aqnqrfheak rteeqlrssq rrdvprttqs tsrpkcaras |
| 721 | cknilacrse elcmecqhpn qrmgpgahrg epapedppkq rcrapacdhf gnakcngycn |
| 781 | ecfqfkqmyg |

By “TNFAIP3 polynucleotide” is meant a polynucleotide encoding an TNFAIP3 polypeptide. An exemplary TNFAIP3 polynucleotide is provided at NCBI Accession No. NM_006290.3. The sequence at NCBI Accession No. NM_006290.3 is provided below:

| SEQ ID NO: 14 | |
|---|---|
| 1 | ctttggaaag tcccgtggaa atccccgggc ctacaacccg catacaactg aaacggggca |
| 61 | aagcagactg cgcagtctgc agtcttcgtg gcgggccaag cgagcttgga gcccgcgggg |
| 121 | gcggagcggt gagagcggcc gccaagagag atcacacccc cagccgaccc tgccagcgag |
| 181 | cgagcccgac cccaggcgtc catggagcgt cgcctccgcc cggtccctgc cccgaccccc |
| 241 | gcctgcggcg cgctcctgcc ttgaccagga cttgggactt tgcgaaagga tcgcggggcc |
| 301 | cggagaggtg ttggagagca caatggctga acaagtcctt cctcaggctt tgtatttgag |
| 361 | caatatgcgg aaagctgtga agatacggga gagaactcca gaagacattt ttaaacctac |
| 421 | taatgggatc attcatcatt ttaaaaccat gcaccgatac acactggaaa tgttcagaac |
| 481 | ttgccagttt tgtcctcagt ttcgggagat catccacaaa gccctcatcg acagaaacat |
| 541 | ccaggccacc ctggaaagcc agaagaaact caactggtgt cgagaagtcc ggaagcttgt |
| 601 | ggcgctgaaa acgaacggtg acggcaattg cctcatgcat gccacttctc agtacatgtg |
| 661 | gggcgttcag gacacagact tggtactgag gaaggcgctg ttcagcacgc tcaaggaaac |
| 721 | agacacacgc aactttaaat tccgctggca actggagtct ctcaaatctc aggaatttgt |
| 781 | tgaaacgggg ctttgctatg atactcggaa ctggaatgat gaatgggaca atcttatcaa |
| 841 | aatggcttcc acagacacac ccatggcccg aagtggactt cagtacaact cactggaaga |
| 901 | aatacacata tttgtccttt gcaacatcct cagaaggcca atcattgtca tttcagacaa |
| 961 | aatgctaaga agtttggaat caggttccaa tttcgcccct ttgaaagtgg gtggaattta |
| 1021 | cttgcctctc cactggcctg cccaggaatg ctacagatac cccattgttc tcggctatga |
| 1081 | cagccatcat tttgtaccct tggtgaccct gaaggacagt gggcctgaaa tccgagctgt |
| 1141 | tccacttgtt aacagagacc ggggaagatt tgaagactta aaagttcact ttttgacaga |
| 1201 | tcctgaaaat gagatgaagg agaagctctt aaaagagtac ttaatggtga tagaaatccc |
| 1261 | cgtccaaggc tgggaccatg gcacaactca tctcatcaat gccgcaaagt tggatgaagc |
| 1321 | taacttacca aaagaaatca atctggtaga tgattacttt gaacttgttc agcatgagta |
| 1381 | caagaaatgg caggaaaaca gcgagcaggg gaggagagag gggcacgccc agaatcccat |
| 1441 | ggaaccttcc gtgccccagc tttctctcat ggatgtaaaa tgtgaaacgc ccaactgccc |
| 1501 | cttcttcatg tctgtgaaca cccagccttt atgccatgag tgctcagaga ggcggcaaaa |
| 1561 | gaatcaaaac aaactcccaa agctgaactc caagccgggc cctgaggggc tccctggcat |
| 1621 | ggcgctcggg gcctctcggg gagaagccta tgagcccttg gcgtggaacc ctgaggagtc |
| 1681 | cactgggggg cctcattcgg ccccaccgac agcacccagc ccttttctgt tcagtgagac |
| 1741 | cactgccatg aagtgcagga gccccggctg ccccttcaca ctgaatgtgc agcacaacgg |
| 1801 | attttgtgaa cgttgccaca acgcccggca acttcacgcc agccacgccc cagaccacac |
| 1861 | aaggcacttg gatcccggga agtgccaagc ctgcctccag gatgttacca ggacatttaa |
| 1921 | tgggatctgc agtacttgct tcaaaaggac tacagcagag gcctcctcca gcctcagcac |
| 1981 | cagcctccct ccttcctgtc accagcgttc caagtcagat ccctcgcggc tcgtccggag |
| 2041 | cccctccccg cattcttgcc acagagctgg aaacgacgcc cctgctggct gcctgtctca |
| 2101 | agctgcacgg actcctgggg acaggacggg gacgagcaag tgcagaaaag ccggctgcgt |
| 2161 | gtattttggg actccagaaa acaagggctt ttgcacactg tgtttcatcg agtacagaga |
| 2221 | aaacaaacat tttgctgctg cctcagggaa agtcagtccc acagcgtcca ggttccagaa |
| 2281 | caccattccg tgcctgggga gggaatgcgg cacccttgga agcaccatgt ttgaaggata |
| 2341 | ctgccagaag tgtttcattg aagctcagaa tcagagattt catgaggcca aaaggacaga |
| 2401 | agagcaactg agatcgagcc agcgcagaga tgtgcctcga accacacaaa gcacctcaag |
| 2461 | gcccaagtgc gcccgggcct cctgcaagaa catcctggcc tgccgcagcg aggagctctg |
| 2521 | catggagtgt cagcatccca accagaggat gggccctggg gcccaccggg gtgagcctgc |
| 2581 | ccccgaagac ccccccaagc agcgttgccg ggcccccgcc tgtgatcatt ttggcaatgc |
| 2641 | caagtgcaac ggctactgca acgaatgctt tcagttcaag cagatgtatg gctaaccgga |
| 2701 | aacaggtggg tcacctcctg caagaagtgg ggcctcgagc tgtcagtcat catggtgcta |
| 2761 | tcctctgaac ccctcagctg ccactgcaac agtgggctta agggtgtctg agcaggagag |
| 2821 | gaaagataag ctcttcgtgg tgcccacgat gctcaggttt ggtaacccgg gagtgttccc |
| 2881 | aggtggcctt agaaagcaaa gcttgtaact ggcaagggat gatgtcagat tcagcccaag |
| 2941 | gttcctcctc tcctaccaag caggaggcca ggaacttctt tggacttgga aggtgtgcgg |
| 3001 | ggactggccg aggcccctgc accctgcgca tcaggactgc ttcatcgtct tggctgagaa |
| 3061 | agggaaaaga cacacaagtc gcgtgggttg gagaagccag agccattcca cctcccctcc |
| 3121 | cccagcatct ctcagagatg tgaagccaga tcctcatggc agcgaggccc tctgcaagaa |
| 3181 | gctcaaggaa gctcagggaa aatggacgta ttcagagagt gtttgtagtt catggttttt |
| 3241 | ccctacctgc ccggttcctt tcctgaggac ccggcagaaa tgcagaacca tccatggact |
| 3301 | gtgattctga ggctgctgag actgaacatg ttcacattga cagaaaaaca agctgctctt |
| 3361 | tataatatgc accttttaaa aaattagaat attttactgg gaagacgtgt aactctttgg |
| 3421 | gttattactg tctttacttc taaagaagtt agcttgaact gaggagtaaa agtgtgtaca |
| 3481 | tatataatat acccttacat tatgtatgag ggattttttt aaattatatt gaaatgctgc |
| 3541 | cctagaagta caataggaag gctaaataat aataacctgt tttctggttg ttgttggggc |
| 3601 | atgagcttgt gtatacactg cttgcataaa ctcaaccagc tgccttttta aagggagctc |
| 3661 | tagtcctttt tgtgtaattc actttattta ttttattaca aacttcaaga ttatttaagt |
| 3721 | gaagatattt cttcagctct ggggaaaatg ccacagtgtt ctcctgagag aacatccttg |
| 3781 | ctttgagtca ggctgtgggc aagttcctga ccacagggag taaattggcc tctttgatac |
| 3841 | acttttgctt gcctccccag gaaagaagga attgcatcca aggtatacat acatattcat |
| 3901 | cgatgtttcg tgcttctcct tatgaaactc cagctatgta ataaaaaact atactctgtg |
| 3961 | ttctgttaat gcctctgagt gtcctacctc cttggagatg agatagggaa ggagcaggga |
| 4021 | tgagactggc aatggtcaca gggaaagatg tggccttttg tgatggtttt attttctgtt |
| 4081 | aacactgtgt cctggggggg ctgggaagtc ccctgcatcc catggtaccc tggtattggg |
| 4141 | acagcaaaag ccagtaacca tgagtatgag gaaatctctt tctgttgctg gcttacagtt |
| 4201 | tctctgtgtg ctttgtggtt gctgtcatat ttgctctaga agaaaaaaaa aaaaggaggg |
| 4261 | gaaatgcatt ttccccagag ataaaggctg ccattttggg ggtctgtact tatggcctga |
| 4321 | aaatatttgt gatccataac tctacacagc ctttactcat actattaggc acactttccc |
| 4381 | cttagagccc cctaagtttt tcccagacga atctttataa tttctttcca aagataccaa |
| 4441 | ataaacttca gtgttttcat ctaattctct taaagttgat atcttaatat tttgtgttga |
| 4501 | tcattatttc cattcttaat gtgaaaaaaa gtaattattt atacttatta taaaaagtat |
| 4561 | ttgaaatttg cacatttaat tgtccctaat agaaagccac ctattctttg ttggatttct |
| 4621 | tcaagttttt ctaaataaat gtaacttttc acaagagtca acattaaaaa ataaattatt |
| 4681 | taagaacaga aaaaaaaaaa aaaaa |

By “TNFRSF12A polypeptide” is meant a polypeptide or fragment thereof having at least about 85% amino acid identity to NCBI Accession No. NP_057723.1 and having a biological activity of a TNFRSF12A (tumor necrosis factor receptor superfamily 12A) polypeptide. Biological activities of a TNFRSF12A polypeptide include TNFSF12/TWEAK receptor activity. The sequence at NCBI Accession No. NP_057723.1 is shown below:

| SEQ ID NO: 15 | |
|---|---|
| 1 | margslrrll rllvlglwla llrsvageqa pgtapcsrgs swsadldkcm dcascrarph |
| 61 | sdfclgcaaa ppapfrllwp ilggalsltf vlgllsgflv wrrcrrrekf ttpieetgge |
| 121 | gcpavaliq |

By “TNFRSF12A polynucleotide” is meant a polynucleotide encoding an TNFRSF12A polypeptide. An exemplary TNFRSF12A polynucleotide is provided at NCBI Accession No. NM_016639.2. The sequence at NCBI Accession No. NM_016639.2 is provided below:

| SEQ ID NO: 16 | |
|---|---|
| 1 | aaggcggggg cgggggcggg gcggcggccg tgggtccctg ccggccggcg gcgggcgcag |
| 61 | acagcggcgg gcgcaggacg tgcactatgg ctcggggctc gctgcgccgg ttgctgcggc |
| 121 | tcctcgtgct ggggctctgg ctggcgttgc tgcgctccgt ggccggggag caagcgccag |
| 181 | gcaccgcccc ctgctcccgc ggcagctcct ggagcgcgga cctggacaag tgcatggact |
| 241 | gcgcgtcttg cagggcgcga ccgcacagcg acttctgcct gggctgcgct gcagcacctc |
| 301 | ctgccccctt ccggctgctt tggcccatcc ttgggggcgc tctgagcctg accttcgtgc |
| 361 | tggggctgct ttctggcttt ttggtctgga gacgatgccg caggagagag aagttcacca |
| 421 | cccccataga ggagaccggc ggagagggct gcccagctgt ggcgctgatc cagtgacaat |
| 481 | gtgccccctg ccagccgggg ctcgcccact catcattcat tcatccattc tagagccagt |
| 541 | ctctgcctcc cagacgcggc gggagccaag ctcctccaac cacaaggggg gtggggggcg |
| 601 | gtgaatcacc tctgaggcct gggcccaggg ttcaggggaa ccttccaagg tgtctggttg |
| 661 | ccctgcctct ggctccagaa cagaaaggga gcctcacgct ggctcacaca aaacagctga |
| 721 | cactgactaa ggaactgcag catttgcaca ggggaggggg gtgccctcct tcctagaggc |
| 781 | cctgggggcc aggctgactt ggggggcaga cttgacacta ggccccactc actcagatgt |
| 841 | cctgaaattc caccacgggg gtcaccctgg ggggttaggg acctattttt aacactaggg |
| 901 | ggctggccca ctaggagggc tggccctaag atacagaccc ccccaactcc ccaaagcggg |
| 961 | gaggagatat ttattttggg gagagtttgg aggggaggga gaatttatta ataaaagaat |
| 1021 | ctttaacttt aaaaaaaaaa aaaaaaaa |

By “TMC4 polypeptide” is meant a polypeptide or fragment thereof having at least about 85% amino acid identity to NCBI Accession No. NP_653287.2 (isoform 2) or NP_001138775.2 (isoform 1) and having a biological activity of a TMC4 (transmembrane channel-like protein 4 isoform 2) polypeptide. Biological activities of a TMC4 polypeptide include ion transport activity. The sequence at NCBI Accession No. NP_653287.2 is shown below:

| SEQ ID NO: 17 | |
|---|---|
| 1 | meenptlese awgssrewla prearggpsl ssvlnelpsa atlryrdpgv lpwgaleeee |
| 61 | edggrsrkaf tevtqtelqd phpsrelpwp mqarrahrqr nasrdqvvyg sgtktdrwar |
| 121 | llrrskektk eglrslqpwa wtlkriggqf gagtesyfsl lrfllllnvl asvlmacmtl |
| 181 | lptwlggapp gppgpdissp cgsynphsqg lvtfatqlfn llsgegylew splfygfypp |
| 241 | rprlavtylc wafavglicl llilhrsvsg lkqtllaese altsyshrvf sawdfglcgd |
| 301 | vhvrlrqrii lyelkvelee tvvrrqaavr tlgqqarvwl vrvllnllvv allgaafygv |
| 361 | ywatgctvel qemplvqelp llklgvnylp sifiagvnfv lppvfkliap legytrsrqi |
| 421 | vfillrtvfl rlaslvvllf slwnqitcgg dseaedcktc gynykqlpcw etvlgqemyk |
| 481 | lllfdlltvl avalliqfpr kllcglcpga lgrlagtqef qvpdevlgli yaqtvvwvgs |
| 541 | ffcpllplln tvkflllfyl kkltlfstcs paartfrasa anfffplvll lglaissvpl |
| 601 | lysiflipps klcgpfrgqs siwaqipesi sslpettqnf lfflgtqafa vplllissil |
| 661 | maytvalans ygrliselkr qreteaqnkv flarravalt stkpal |

By “TMC4 polynucleotide” is meant a polynucleotide encoding a TMC4 polypeptide. An exemplary TMC4 polynucleotide sequence is provided at NCBI Accession No. NM_144686.3. The exemplary sequence provided at NCBI Accession No. NM_144686.3 is reproduced below:

| SEQ ID NO: 18 | |
|---|---|
| 1 | ctgagaaacc acaggaagtg taccttactc cctccgggcc acctgctggc caggtacaca |
| 61 | cctgcccctg gcccctccct tacctggggc agtgtctgcc tggtggccac tagagacagc |
| 121 | ccagcctggg gccatggaag aaaacccgac cttggaatca gaagcctggg gctcctctag |
| 181 | ggagtggctg gccccccggg aggccagagg aggcccatcg ctgtcttctg tgctgaacga |
| 241 | gctgcccagt gctgccaccc ttcggtaccg agaccctggg gtgctgcctt ggggggcgct |
| 301 | ggaggaggag gaggaggatg gaggaaggag cagaaaggcc ttcacagaag tcacccagac |
| 361 | agagctgcag gaccctcacc cttcccggga actgccctgg cccatgcagg ccagacgggc |
| 421 | acacaggcaa agaaatgcca gcagggacca ggtggtctat ggctctggaa ctaagacgga |
| 481 | ccgatgggcg cggctacttc ggaggtccaa ggagaaaaca aaggaaggct tgcgaagcct |
| 541 | gcagccctgg gcgtggacac tgaagaggat cgggggccag tttggcgccg gcacggagtc |
| 601 | ctacttctcc ctgctgcgct tcctgctcct tcttaacgtg ctggcctctg tgctcatggc |
| 661 | ctgcatgacg ctgctgccca cctggttggg aggcgctccc ccaggccctc ccggccccga |
| 721 | catctcctcg ccctgcggct cctataaccc ccactcccag ggcctggtca cctttgccac |
| 781 | ccagctcttc aacttgctct cgggtgaggg ttacctggaa tggtcccctc tcttctatgg |
| 841 | cttctacccg ccccgcccac gcctggcggt cacctacctg tgctgggcct ttgccgttgg |
| 901 | cctcatctgc ctcctgctca tcctgcatcg ctcggtgtct gggctgaagc agacactgct |
| 961 | ggcggagtcc gaggctctga ccagctacag ccaccgggtg ttctcggcct gggacttcgg |
| 1021 | tctctgcggg gacgtccacg tgcggctgcg ccagcgcatc atcttgtacg aattaaaggt |
| 1081 | ggagctggag gagacagtgg tgcggcgcca ggctgcggtg cggacgctgg gccagcaagc |
| 1141 | cagggtttgg ttggtgcggg tgctgctcaa cctgctggtg gtcgcgctcc tgggggcagc |
| 1201 | cttctatggc gtctactggg ctacggggtg caccgtggag ctgcaggaga tgccccttgt |
| 1261 | ccaggagttg ccactgctga agcttggggt gaattacctt ccgtccatct tcatcgctgg |
| 1321 | ggtcaatttt gtgctgccgc ccgtgttcaa gctcattgct ccactggagg gctacactcg |
| 1381 | gagtcgccag atcgttttta tcctgctcag gaccgtgttt cttcgcctcg cctccctggt |
| 1441 | ggtcctgctc ttctctctct ggaatcagat cacttgtggg ggcgactccg aggctgagga |
| 1501 | ctgcaaaacc tgtggctaca attacaaaca acttccgtgc tgggagactg tcctgggcca |
| 1561 | ggaaatgtac aaacttctgc tctttgatct gctgactgtc ttggcagtcg cgctgctcat |
| 1621 | ccagtttcct agaaagctcc tctgtggcct ctgtcctggg gcgctgggtc gtctggcggg |
| 1681 | gacccaagag ttccaggtgc ccgacgaggt gctggggctc atctacgcgc agacggtggt |
| 1741 | ctgggtgggg agttttttct gccctttact gcccctgctt aacacggtca agttcctgct |
| 1801 | gcttttctac ctgaagaagc ttaccctctt ctccacctgc tccccggctg cccgcacctt |
| 1861 | ccgggcctcc gcggcgaatt tctttttccc cttggtcctt ctcctgggtc tggccatctc |
| 1921 | cagcgttccc ctgctttaca gcatcttcct gatcccgcct tctaagctgt gtggtccatt |
| 1981 | ccgggggcag tcgtccatct gggcccagat ccctgagtct atttccagcc tccctgagac |
| 2041 | cacccagaat ttcctcttct tcctggggac ccaggctttt gctgtgcccc ttctgctgat |
| 2101 | ctccagcatc ctgatggcgt acactgtggc tctggctaac tcctacggac gcctcatctc |
| 2161 | tgagctcaaa cgtcagagag agacggaggc gcagaataaa gtcttcctgg cacggcgcgc |
| 2221 | tgtggcgctg acctccacca aaccggctct ttgacccccg cagcccacgt cccgctttca |
| 2281 | gaccccaggc ccattgtaag cctaggtcac aacatctgta aactaggaga actggagaag |
| 2341 | actccacgcc cttccagctt tggtatctgg agatttccag ggcccctcgc cgccacgtcc |
| 2401 | ctgactctcg ggtgatcttc cttgtatcaa taaatacagc cgaggttgct gagcgcgctt |
| 2461 | tgaaaaaaaa aaaaaaa |

A “therapeutic” treatment is a treatment administered to a subject who exhibits a sign or symptom of pathology, for the purpose of diminishing or eliminating that sign or symptom.

As used herein, “treating a disease or disorder” means reducing the frequency with which a sign or symptom of the disease or disorder is experienced by a patient.

The phrase “therapeutically effective amount,” as used herein, refers to an amount that is sufficient or effective to prevent or treat (delay or prevent the onset of, prevent the progression of, inhibit, decrease or reverse) a disease or disorder associated with ASD, including alleviating signs and symptoms of such diseases or disorders.

By “variant” as is meant a polynucleotide or polypeptide sequence that differs from a wild-type or reference sequence by one or more nucleotides or one or more amino acids.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Description

The present invention relates to the identification of blood-based biomarkers that are associated with an ASD-associated inflammatory disorder. The invention is based at least in part on the comparison of gene expression profiles (differentially expressed transcripts) in inflamed ileocolonic tissues from GI symptomatic ASD children and in peripheral blood of ASD children. In an earlier study, the molecular profile in gastrointestinal mucosal biopsies from ASD^{IC+} children was found to yield unique differentially expressed transcripts, not present in pediatric Crohn's disease (CD) or ulcerative colitis (UC). In studies described herein, the mucosal expression profile is compared to the peripheral blood expression profile in a second cohort of cases and controls. In two separate case/control mucosal-based cohorts, an overlap of 59 differentially expressed transcripts that are unique to inflamed ileocolonic tissues from GI symptomatic ASD children was found. Nine of these 59 transcripts are also differentially expressed in the peripheral blood of ASD children. These nine transcripts therefore represent a blood-based biomarker for ASD-associated ileocolonic inflammation.

In one embodiment, the biomarkers of the invention are useful for discriminating between different inflammatory disorders.

Such biomarkers could be used for ASD-associated inflammatory disorder screening and diagnosis, as well as potentially for designing novel pharmaceuticals that would target the genes responsible for the DETs, and in assessing response to new therapies. Given the probability of multiple underlying pathogenic mechanisms of some ASD-associated inflammatory disorders, the present invention provides novel biomarkers present in a blood sample of a subject. The biomarkers of the invention allow a more accurate diagnosis, prognosis, or treatment strategy of an ASD-associated inflammatory disorder.

In one embodiment, the present invention includes a method of screening for an Autism Spectrum Disorder in a patient by analyzing differential gene expression patterns comprising the steps of: obtaining a nucleic acid sample from cells of a patient; performing a nucleic acid analysis on the nucleic acid samples to obtain a gene expression analysis data set; and comparing said data set to a control data set corresponding to a gene ensemble of differentially expressed genes indicative of autism spectrum disorder, wherein autism spectrum disorder is indicated upon observing statistically significant differential gene expression. In one embodiment, the nucleic acid sample is obtained from a blood sample of a patient.

In some embodiments of the invention, one can observe an expression profile of at least, 2, 3, 4, 5, 6, 7, 8, 9 or 10 or more genes whose expression is shown to be dysregulated in autism spectrum disorders (e.g. using microarray technologies). In certain embodiments of the invention, the method is performed on a plurality of individuals and the results are then categorized based upon similarities or differences in their gene expression profiles. Optionally, the expression profile(s) is observed and/or collected and/or stored using a computer system comprising a processor element and a memory storage element adapted to process and store data from one or more expression profiles (e.g. in a library of such profiles). In this context, certain embodiments of the invention comprise an electronically searchable library of profiles, wherein the profiles include an individual's gene expression data in combination with other diagnostic data, for example assessments of behavior associated with autism spectrum disorders.

In one embodiment, the invention relates to one or more biomarkers of ASD. The invention is partly based upon the discovery of differentially expressed transcripts (DETs) detected in samples obtained from subjects with ASD^{IC}, compared to samples obtained from either non-disease control subjects, subjects with Crohn's disease, or subjects with ulcerative colitis. Given the broad heterogeneity that is the hallmark of ASD, coupled with the understanding that earlier diagnosis and treatment provides the greatest chance for the most positive outcomes, a blood-based test to diagnose autism (and/or ASD subtypes) early on would have tremendous clinical value. A key confound in these efforts, heterogeneity (in the form of ASD subtypes) exists within the core domains (language, social interaction, and range of interests), but also within associated medical comorbidity domains (e.g. epilepsy, sleep disorders, gastrointestinal disorders, etc.). Without being bound by theory, it is believed the most promising validated blood-based biomarkers would be derived within ASD subtypes. It is an ASD subtype within the comorbidity domains, i.e. gastrointestinal disorders, that is the focus of the studies described herein.

In one embodiment, the present invention includes a method of treating a pre-selected patient, comprising the step of administering a treatment for a gastrointestinal disorder. The patient is pre-selected by detecting an alteration in the level of a polynucleotide or polypeptide of at least one biomarker of the invention.

In one embodiment, the biomarker of the invention is FCER1A, CYP2S1, TMC4, TNFRSF12A, IL1RN, TNFAIP3, CENPE, MTHFD2, or SIGLEC17P. In one embodiment, the biomarker of the invention is detected to have decreased expression in a subject with ASD compared to a control sample. In another embodiment, the biomarker of the invention is detected to have increased expression in a subject with ASD compared to a control sample.

### Methods of Treatment

The methods of the invention include methods of treating gastrointestinal disorders or ASD-associated inflammatory disorders in pre-selected patients. In one embodiment, a patient is pre-selected by detecting an alteration in the level of a polynucleotide or polypeptide of any one or more of biomarkers FCER1A, CYP2S1, TMC4, TNFRSF12A, IL1RN, TNFAIP3, CENPE, MTHFD2, or SIGLEC17P relative to a reference. In a particular embodiment, a patient is pre-selected by detecting an increase in the level of TMC4 polypeptide or polynucleotide, an increase in the level of TNFAIP3 polypeptide or polynucleotide, a decrease in the level of FCER1A polypeptide or polynucleotide, a decrease in the level of CYP2S1 polypeptide or polynucleotide, a decrease in the level of TNFRSF12A polypeptide or polynucleotide, a decrease in the level of IL1RN polypeptide or polynucleotide, a decrease in the level of CENPE polypeptide or polynucleotide, a decrease in the level of MTHFD2 polypeptide or polynucleotide, or a decrease in the level of SIGLEC17P polypeptide or polynucleotide, relative to a reference. Methods of detecting levels of polynucleotides or polypeptides of the biomarkers of the invention are described elsewhere herein.

The methods include the step of administering a treatment for gastrointestinal disorders or ASD-associated inflammatory disorders to a pre-selected subject. Treatments for such gastrointestinal disorders or ASD-associated inflammatory disorders include, without limitation, treatment with therapeutic agents such as corticosteroids, immunomodulators, 5-aminosalicylic acid preparations, cytokine specific antagonists, antimicrobials, probiotics, and supplemental digestive enzymes. Exemplary corticosteroids include, without limitation, budesonide, dexamethasone, hydrocortisone, methylprednisolone, prednisolone, and prednisone. Examples of immunomodulators include, without limitation, azathioprine, mercaptopurine, and methotrexate. Treatment for gastrointestinal disorders or ASD-associated inflammatory disorders also includes dietary restrictions. Additionally, treatment for such gastrointestinal disorders or ASD-associated inflammatory disorders may include targeted therapies that decrease or eliminate the expression of any of the polynucleotides or polypeptides of the genes responsible for the unique biomarker profiles described herein.

In one embodiment, a therapeutically effective amount of a composition comprising the therapeutic agent is administered to the subject that is sufficient to treat the gastrointestinal disorder or ASD-associated inflammatory disorder, or symptom thereof, under conditions such that the disorder is treated. The composition may be administered systemically, for example, formulated in a pharmaceutically-acceptable buffer such as physiological saline. The pharmaceutical composition may be administered parenterally by injection, infusion or implantation (subcutaneous, intravenous, intramuscular, intraperitoneal, or the like) in dosage forms, formulations, or via suitable delivery devices or implants containing conventional, non-toxic pharmaceutically acceptable carriers and adjuvants. The formulation and preparation of such compositions are well known to those skilled in the art of pharmaceutical formulation. Formulations can be found in Remington: The Science and Practice of Pharmacy. In particular embodiments, the treatment is administered orally, intravenously, topically, by enema, or by suppository.

The amount of the therapeutic agent to be administered varies depending upon the manner of administration, the age and body weight of the patient, and with the clinical symptoms of the gastrointestinal disorder or ASD-associated inflammatory disorder. Generally, amounts will be in the range of those used for other agents used in the treatment of gastrointestinal disorders or ASD-associated inflammatory disorders, although in certain instances lower amounts will be needed because of the increased specificity of the agent. A composition is administered at a dosage that decreases effects or symptoms of a gastrointestinal disorder or ASD-associated inflammatory disorder as determined by a method known to one skilled in the art.

### Diagnostic Methods

The methods of the invention also include the use of a biomarker to detect a gastrointestinal disorder and/or autism spectrum disorder in a subject (e.g., a human subject). As described herein, altered gene expression of transcripts indicated the presence of a gastrointestinal disorder in ASD individuals. The molecular evidence indicated an overlapping, yet unique, IBD-like condition in ASD children.

Biological samples include tissue samples (e.g., cell samples, biopsy samples), such as tissue from colon or ileum. Biological samples also include bodily fluids, including, but not limited to, blood, blood serum, plasma, saliva, and urine. Altered levels of a biomarker alone or in combination with one or more additional markers relative to a reference are considered a positive indicator of a gastrointestinal and/or autism spectrum disorder. In one embodiment, the biological sample is blood.

Any suitable method can be used to detect one or more of the markers described herein. Successful practice of the invention can be achieved with one or a combination of methods that can detect and, preferably, quantify the markers. These methods include, without limitation, hybridization-based methods, including those employed in biochip arrays, mass spectrometry (e.g., laser desorption/ionization mass spectrometry), fluorescence (e.g. sandwich immunoassay), surface plasmon resonance, ellipsometry and atomic force microscopy. Expression levels of markers (e.g., polynucleotides or polypeptides) are compared by procedures well known in the art, such as RT-PCR, Northern blotting, Western blotting, flow cytometry, immunocytochemistry, binding to magnetic and/or antibody-coated beads, in situ hybridization, fluorescence in situ hybridization (FISH), flow chamber adhesion assay, ELISA, microarray analysis, or colorimetric assays. Methods may further include, one or more of electrospray ionization mass spectrometry (ESI-MS), ESI-MS/MS, ESI-MS/(MS) n, matrix-assisted laser desorption ionization time-of-flight mass spectrometry (MALDI-TOF-MS), surface-enhanced laser desorption/ionization time-of-flight mass spectrometry (SELDI-TOF-MS), desorption/ionization on silicon (DIOS), secondary ion mass spectrometry (SIMS), quadrupole time-of-flight (Q-TOF), atmospheric pressure chemical ionization mass spectrometry (APCI-MS), APCI-MS/MS, APCI-(MS)″, atmospheric pressure photoionization mass spectrometry (APPI-MS), APPI-MS/MS, and APPI-(MS) n, quadrupole mass spectrometry, fourier transform mass spectrometry (FTMS), and ion trap mass spectrometry, where n is an integer greater than zero. Detection methods may include use of a biochip array. Biochip arrays useful in the invention include protein and polynucleotide arrays. One or more markers are captured on the biochip array and subjected to analysis to detect the level of the markers in a sample.

Markers may be captured with capture reagents immobilized to a solid support, such as a biochip, a multiwell microtiter plate, a resin, or a nitrocellulose membrane that is subsequently probed for the presence or level of a marker. Capture can be on a chromatographic surface or a biospecific surface. For example, a sample containing the markers, such as serum, may be used to contact the active surface of a biochip for a sufficient time to allow binding. Unbound molecules are washed from the surface using a suitable eluant, such as phosphate buffered saline. In general, the more stringent the eluant, the more tightly the proteins must be bound to be retained after the wash.

Upon capture on a biochip, analytes can be detected by a variety of detection methods selected from, for example, a gas phase ion spectrometry method, an optical method, an electrochemical method, atomic force microscopy and a radio frequency method. In one embodiment, mass spectrometry, and in particular, SELDI, is used. Optical methods include, for example, detection of fluorescence, luminescence, chemiluminescence, absorbance, reflectance, transmittance, birefringence or refractive index (e.g., surface plasmon resonance, ellipsometry, a resonant mirror method, a grating coupler waveguide method or interferometry). Optical methods include microscopy (both confocal and non-confocal), imaging methods and nonimaging methods. Immunoassays in various formats (e.g., ELISA) are popular methods for detection of analytes captured on a solid phase. Electrochemical methods include voltametry and amperometry methods. Radio frequency methods include multipolar resonance spectroscopy.

Mass spectrometry (MS) is a well-known tool for analyzing chemical compounds. Thus, in one embodiment, the methods of the present invention comprise performing quantitative MS to measure the serum peptide marker. The method may be performed in an automated (Villanueva, et al., Nature Protocols (2006) 1(2):880-891) or semi-automated format. This can be accomplished, for example with MS operably linked to a liquid chromatography device (LC-MS/MS or LC-MS) or gas chromatography device (GC-MS or GC-MS/MS). Methods for performing MS are known in the field and have been disclosed, for example, in US Patent Application Publication Nos: 20050023454; 20050035286; U.S. Pat. No. 5,800,979 and references disclosed therein.

In an additional embodiment of the methods of the present invention, multiple markers are measured. The use of multiple markers increases the predictive value of the test and provides greater utility in diagnosis, patient stratification and patient monitoring. The process called “Pattern recognition” detects the patterns formed by multiple markers greatly improves the sensitivity and specificity of clinical markers for predictive medicine. Subtle variations in data from clinical samples indicate that certain patterns of protein expression can predict phenotypes such as the presence or absence of a certain disease, a particular stage of disease-progression, or a positive or adverse response to drug treatments. In the present invention, additional markers may include cytokine profiles and/or serum antibodies directed against gut microbes.

Expression levels of particular nucleic acids or polypeptides are correlated with a gastrointestinal disorder and/or autistic spectrum disorder and thus are useful in diagnosis. Methods for measuring levels of polypeptide include immunoassay, ELISA, western blotting and radioimmunoassay. Oligonucleotides or longer fragments derived from a nucleic acid sequence described herein, antibodies that bind a polypeptide described herein, or any other method known in the art may be used to monitor expression of a polynucleotide or polypeptide of interest. In other embodiments, a 1.5, 2, 3, 4, 5, or 6-fold change in the level of a marker of the invention is indicative of a gastrointestinal and/or autistic spectrum disorder. In yet another embodiment, an expression profile that characterizes alterations in the expression two or more markers is correlated with a particular disease state (e.g., gastrointestinal and/or autistic spectrum disorder).

The diagnostic methods described herein can be used individually or in combination with any other diagnostic method described herein for a more accurate diagnosis of the presence of a gastrointestinal and/or autistic spectrum disorder. The diagnostic methods described herein can also be used to reliably distinguish ileoclitis in an individual having autism spectrum disorder from Crohn's disease or inflammatory bowel disease.

As indicated above, the invention provides methods for aiding diagnosis of a gastrointestinal and/or autism spectrum disorder using one or more markers, as specified herein. These markers can be used alone, in combination with other markers in any set, or with entirely different markers in aiding diagnosis. The measurement of markers may also involve quantifying the markers to correlate the detection of markers with a diagnosis of a gastrointestinal and/or autism spectrum disorder. Thus, if the amount of the markers detected in a subject being tested is different compared to a control amount (i.e., higher than the control), then the subject being tested has a higher probability of having a gastrointestinal and/or autism spectrum disorder. The correlation may take into account the amount of the marker or markers in the sample compared to a control amount of the marker or markers (e.g., in normal subjects). A control can be, e.g., the average or median amount of marker present in comparable samples of normal subjects in normal subjects or in subjects such as where the disease or disorder is undetectable. The control amount is measured under the same or substantially similar experimental conditions as in measuring the test amount. As a result, the control can be employed as a reference standard, where the normal (non-disease) phenotype is known, and each result can be compared to that standard, rather than re-running a control.

Accordingly, a marker profile may be obtained from a subject sample and compared to a reference marker profile obtained from a reference population, so that it is possible to classify the subject as belonging to or not belonging to the reference population. The correlation may take into account the presence or absence of the markers in a test sample and the frequency of detection of the same markers in a control. The correlation may take into account both of such factors to facilitate diagnosis of a gastrointestinal and/or austism spectrum disorder.

In certain embodiments of the methods of qualifying a disorder, the methods further comprise managing subject treatment based on the status. The invention also provides for such methods where the markers (or specific combination of markers) are measured again after subject management. In these cases, the methods are used to monitor the status of the disorder or progression of the disorder.

Any marker, individually, is useful in aiding in the diagnosis of a gastrointestinal and/or autistic spectrum disorder. First, the selected marker is detected in a subject sample using the methods described herein. Then, the result is compared with a control that distinguishes disorder status from non-disorder status. As is well understood in the art, the techniques can be adjusted to increase sensitivity or specificity of the diagnostic assay depending on the preference of the diagnostician.

The diagnosis of a gastrointestinal and/or autistic spectrum disorder can be used to inform treatment selection. Treatments for such ASD or ASD inflammatory disorders include without limitation treatment with corticosteroids, immunomodulators, 5-aminosalicylic acid preparations, cytokine specific antagonists, dietary restrictions, antimicrobials, probiotics, and supplemental digestive enzymes. Additionally, treatment for such ASD or ASD inflammatory disorders may include targeted therapies that decrease or eliminate the expression of any of the nucleic acid molecules or polypeptides of the genes responsible for the unique transcriptomes described herein.

While individual markers are useful diagnostic markers, in some instances, a combination of markers provides greater predictive value than single markers alone. The detection of a plurality of markers (or absence thereof, as the case may be) in a sample can increase the percentage of true positive and true negative diagnoses and decrease the percentage of false positive or false negative diagnoses. Thus, preferred methods of the present invention comprise the measurement of more than one marker.

### Microarrays

As reported herein, a number of biomarkers have been identified that are associated with a gastrointestinal and/or autistic spectrum disorder. In particular, the invention provides diagnostic methods and compositions useful for identifying an expression profile that identifies a subject as having a gastrointestinal and/or autistic spectrum disorder. Such assays can be used to measure an alteration in the level of a gene transcipt or polypeptide encoded by the transcript.

The polypeptides and polynucleotides of the invention are useful as hybridizable array elements in a microarray. The array elements are organized in an ordered fashion such that each element is present at a specified location on the substrate. Useful substrate materials include membranes, composed of paper, nylon or other materials, filters, chips, glass slides, and other solid supports. The ordered arrangement of the array elements allows hybridization patterns and intensities to be interpreted as expression levels of particular genes or proteins. Methods for making nucleic acid microarrays are known to the skilled artisan and are described, for example, in U.S. Pat. No. 5,837,832, Lockhart, et al. (Nat. Biotech. 14: 1675-1680, 1996), and Schena, et al. (Proc. Natl. Acad. Sci. 93: 10614-10619, 1996), herein incorporated by reference. Methods for making polypeptide microarrays are described, for example, by Ge (Nucleic Acids Res. 28: e3..i-e3. vii, 2000), MacBeath et al., (Science 289: 1760-1763, 2000), Zhu et al. (Nature Genet. 26:283-289), and in U.S. Pat. No. 6,436,665, hereby incorporated by reference.

### Nucleic Acid Microarrays

To produce a nucleic acid microarray, oligonucleotides may be synthesized or bound to the surface of a substrate using a chemical coupling procedure and an ink jet application apparatus, as described in PCT application W095/251116 (Baldeschweiler et al.), incorporated herein by reference. Alternatively, a gridded array may be used to arrange and link cDNA fragments or oligonucleotides to the surface of a substrate using a vacuum system, thermal, UV, mechanical or chemical bonding procedure.

A nucleic acid molecule (e.g. RNA or DNA) derived from a biological sample may be used to produce a hybridization probe as described herein. The biological samples are generally derived from a patient, preferably as a bodily fluid (such as blood, blood serum, plasma, saliva, urine, seminal fluids, and ejaculate) or tissue sample (e.g. a tissue sample obtained by biopsy). For some applications, cultured cells or other tissue preparations may be used. The mRNA is isolated according to standard methods, and cDNA is produced and used as a template to make complementary RNA suitable for hybridization. Such methods are known in the art. The RNA is amplified in the presence of fluorescent nucleotides, and the labeled probes are then incubated with the microarray to allow the probe sequence to hybridize to complementary oligonucleotides bound to the microarray.

Incubation conditions are adjusted such that hybridization occurs with precise complementary matches or with various degrees of less complementarity depending on the degree of stringency employed. For example, stringent salt concentration will ordinarily be less than about 750 mM NaCl and 75 mM trisodium citrate, preferably less than about 500 mM NaCl and 50 mM trisodium citrate, and most preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and most preferably at least about 50% formamide. Stringent temperature conditions will ordinarily include temperatures of at least about 30° C., more preferably of at least about 37° C., and most preferably of at least about 42° C.

Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/μl denatured salmon sperm DNA (ssDNA). In a most preferred embodiment, hybridization will occur at 42° C. in 250 mM NaCl, 25 mM trisodium citrate, 1% SDS, 50% formamide, and 200 g/ml ssDNA. Useful variations on these conditions will be readily apparent to those skilled in the art.

The removal of nonhybridized probes may be accomplished, for example, by washing. The washing steps that follow hybridization can also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and most preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42° C. in 15 mM NaCl, 1.5 mM trisodium citrate, and 0.1% SDS. In a most preferred embodiment, wash steps will occur at 68° C. in 15 mM NaCl, 1.5 mM trisodium citrate, and 0.1% SDS. Additional variations on these conditions will be readily apparent to those skilled in the art.

A detection system may be used to measure the absence, presence, and amount of hybridization for all of the distinct nucleic acid sequences simultaneously (e.g., Heller et al., Proc. Natl. Acad. Sci. 94:2150-2155, 1997). Preferably, a scanner is used to determine the levels and patterns of fluorescence.

### Polymerase Chain Reaction (PCR)

Polymerase chain reaction (PCR) is widely known in the art. For example, U.S. Pat. Nos. 4,683,195, 4,683,202, and 4,800,159; K. Mullis, Cold Spring Harbor Symp. Quant. Biol., 51:263-273 (1986); and C. R. Newton & A. Graham, Introduction to Biotechniques: PCR, 2nd Ed., Springer-Verlag (New York: 1997), the disclosures of which are incorporated herein by reference, describe processes to amplify a nucleic acid sample target using PCR amplification extension primers which hybridize with the sample target. As the PCR amplification primers are extended, using a DNA polymerase (preferably thermostable), more sample target is made so that more primers can be used to repeat the process, thus amplifying the sample target sequence. Typically, the reaction conditions are cycled between those conducive to hybridization and nucleic acid polymerization, and those that result in the denaturation of duplex molecules.

In the first step of the reaction, the nucleic acid molecules of the sample are transiently heated, and then cooled, in order to denature double stranded molecules. Forward and reverse primers are present in the amplification reaction mixture at an excess concentration relative to the sample target. When the sample is incubated under conditions conducive to hybridization and polymerization, the primers hybridize to the complementary strand of the nucleic acid molecule at a position 3′ to the sequence of the region desired to be amplified that is the complement of the sequence whose amplification is desired. Upon hybridization, the 3′ ends of the primers are extended by the polymerase. The extension of the primer results in the synthesis of a DNA molecule having the exact sequence of the complement of the desired nucleic acid sample target. The PCR reaction is capable of exponentially amplifying the desired nucleic acid sequences, with a near doubling of the number of molecules having the desired sequence in each cycle. Thus, by permitting cycles of hybridization, polymerization, and denaturation, an exponential increase in the concentration of the desired nucleic acid molecule can be achieved.

The methods of the present invention involve amplifying regions of a polynucleotide with high fidelity using a thermostable DNA polymerase having 3′→5′ exonuclease activity. As defined herein, “3′→5′ exonuclease activity” refers to the activity of a template-specific nucleic acid polymerase having a 3′→5′ exonuclease activity associated with some DNA polymerases, in which one or more nucleotides are removed from the 3′ end of an oligonucleotide in a sequential manner. Polymerase enzymes having high fidelity 3′→5′ exonuclease activity are useful, for example, when primer extension must be performed with high specificity. Polymerase enzymes having 3′→5′ exonuclease proofreading activity are known to those in the art. Examples of suitable proofreading enzymes include TaKaRa LA Taq (Takara Shuzo Co., Ltd.) and Pfu (Stratagene), Vent, Deep Vent (New England Biolabs). Exemplary methods for performing long range PCR are disclosed, for example, in U.S. Pat. No. 5,436,149; Barnes, Proc. Natl. Acad. Sci. USA 91:2216-2220 (1994); Tellier et al., Methods in Molecular Biology, Vol. 226, PCR Protocols, 2nd Edition, pp. 173-177; and, Cheng et al., Proc. Natl. Acad. Sci. 91:5695-5699 (1994); the contents of which are incorporated herein by reference. In various embodiments, long range PCR involves one DNA polymerase. In some embodiments, long range PCR may involve more than one DNA polymerase. When using a combination of polymerases in long range PCR, it is preferable to include one polymerase having 3′→5′ exonuclease activity, which assures high fidelity generation of the PCR product from the DNA template. Typically, a non-proofreading polymerase, which is the main polymerase is also used in conjunction with the proofreading polymerase in long range PCR reactions. Long range PCR can also be performed using commercially available kits, such as LA PCR kit available from Takara Bio Inc.

### Hybridization

There are a variety of ways by which one can assess genetic profiles, and many of these rely on nucleic acid hybridization. Hybridization is defined as the ability of a nucleic acid to selectively form duplex molecules with complementary stretches of DNAs and/or RNAs. Depending on the application envisioned, one would employ varying conditions of hybridization to achieve varying degrees of selectivity of the probe or primers for the target sequence.

Typically, a probe or primer of between 13 and 100 nucleotides, preferably between 17 and 100 nucleotides in length up to 1-2 kilobases or more in length will allow the formation of a duplex molecule that is both stable and selective. Such fragments may be readily prepared, for example, by directly synthesizing the fragment by chemical means or by introducing selected sequences into recombinant vectors for recombinant production.

For applications requiring high selectivity, one will typically desire to employ relatively high stringency conditions to form the hybrids. For example, relatively low salt and/or high temperature conditions, such as provided by about 0.02 M to about 0.10 M NaCl at temperatures of about 50° C. to about 70° C. Such high stringency conditions tolerate little, if any, mismatch between the probe or primers and the template or target strand and would be particularly suitable for isolating specific genes or for detecting specific mRNA transcripts. It is generally appreciated that conditions can be rendered more stringent by the addition of increasing amounts of formamide.

For certain applications, for example, lower stringency conditions may be used. Under these conditions, hybridization may occur even though the sequences of the hybridizing strands are not perfectly complementary, but are mismatched at one or more positions.

Conditions may be rendered less stringent by increasing salt concentration and/or decreasing temperature. For example, a medium stringency condition could be provided by about 0.1 to 0.25 M NaCl at temperatures of about 37° C. to about 55° C., while a low stringency condition could be provided by about 0.15 M to about 0.9 M salt, at temperatures ranging from about 20° C. to about 55° C. Hybridization conditions can be readily manipulated depending on the desired results.

In other embodiments, hybridization may be achieved under conditions of, for example, 50 mM Tris-HCl (pH 8.3), 75 mM KCl, 3 mM MgCl₂, 1.0 mM dithiothreitol, at temperatures between approximately 20° C. to about 37° C. Other hybridization conditions utilized could include approximately 10 mM Tris-HCl (pH 8.3), 50 mM KCl, 1.5 mM MgCl₂, at temperatures ranging from approximately 40° C. to about 72° C.

In certain embodiments, it will be advantageous to employ nucleic acids of defined sequences of the present invention in combination with an appropriate means, such as a label, for determining hybridization. A wide variety of appropriate indicator means are known in the art, including fluorescent, radioactive, enzymatic or other ligands, such as avidin/biotin, which are capable of being detected. In preferred embodiments, one may desire to employ a fluorescent label or an enzyme tag such as urease, alkaline phosphatase or peroxidase, instead of radioactive or other environmentally undesirable reagents. In the case of enzyme tags, colorimetric indicator substrates are known that can be employed to provide a detection means that is visibly or spectrophotometrically detectable, to identify specific hybridization with complementary nucleic acid containing samples.

In general, it is envisioned that the probes or primers described herein will be useful as reagents in solution hybridization, as in PCR, for detection of expression of corresponding genes, as well as in embodiments employing a solid phase. In embodiments involving a solid phase, the test DNA (or RNA) is adsorbed or otherwise affixed to a selected matrix or surface. This fixed, single-stranded nucleic acid is then subjected to hybridization with selected probes under desired conditions. The conditions selected will depend on the particular circumstances (depending, for example, on the G+C content, type of target nucleic acid, source of nucleic acid, size of hybridization probe, etc.). Optimization of hybridization conditions for the particular application of interest is well known to those of skill in the art. After washing of the hybridized molecules to remove non-specifically bound probe molecules, hybridization is detected, and/or quantified, by determining the amount of bound label. Representative solid phase hybridization methods are disclosed in U.S. Pat. Nos. 5,843,663, 5,900,481 and 5,919,626. Other methods of hybridization that may be used in the practice of the present invention are disclosed in U.S. Pat. Nos. 5,849,481, 5,849,486 and 5,851,772. The relevant portions of these and other references identified in this section of the Specification are incorporated herein by reference.

### Kits

The present compositions may be assembled into kits or pharmaceutical systems for use in detecting or diagnosing a gastrointestinal disorder and/or austim spectrum disorder. Materials and reagents required for measuring the level of a polynucleotide or polypeptide in a sample may be assembled together in a kit. This generally will comprise a capture reagent, primer, or probe designed to hybridize specifically to target polynucleotides of interest. The primer or probe may be labeled with a radioisotope, a fluorophore, a chromophore, a dye, an enzyme, or TOF carrier. Also included may be enzymes suitable for amplifying nucleic acids, including various polymerases (reverse transcriptase, Taq, etc.), dNTPs/rNTPs and buffers (e.g., 10×buffer=100 mM Tris-HCl (pH 8.3), and 500 mM KC1) to provide the necessary reaction mixture for amplification. One or more of the deoxynucleotides may be labeled with a radioisotope, a fluorophore, a chromophore, a dye, or an enzyme. Such kits may also include enzymes and other reagents suitable for detection of specific nucleic acids or amplification products. The kits of the invention may also comprise associated instructions for using the agents of the invention. Additionally, one or more agents for treating a gastrointestinal disorder or autism spectrum disorder may be included.

Kits according to this aspect of the invention comprise a carrier means, such as a box, carton, tube or the like, having in close confinement therein one or more container means, such as vials, tubes, ampoules, bottles and the like. The container means of the kits will generally include at least one vial, test tube, flask, bottle, or other container means, into which a component may be placed, and preferably, suitably aliquoted. Where there is more than one component in the kit, the kit also will generally contain additional containers into which the additional components may be separately placed. However, various combinations of components may be comprised in a container. The kits of the present invention also will typically include a means for packaging the component containers in close confinement for commercial sale. Such packaging may include injection or blow-molded plastic containers into which the desired component containers are retained.

### In General

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### Example 1: Identification of a Blood-Based Biomarker in Patients with Autism Spectrum Disorder Associated Ileocolitis

Gastrointestinal (GI) symptoms are common in children with autism spectrum disorder (ASD) with a significant proportion of GI symptomatic ASD children having histologic ileocolitis (inflammation of the terminal ileum and/or colon). Previously, the molecular characterization of GI biopsy tissue from ASD children with ileocolitis (ASD^{IC+}) compared to anatomically similar inflamed mucosal GI tissue from typically developing (TD) children with inflammatory bowel disease (IBD; i.e. Crohn's disease or ulcerative colitis) and TD GI symptomatic children without evidence of GI mucosal inflammation (TD^{IC-}) was reported. In that study, it was found that inflamed ileocolonic tissue in ASD children possess a gene expression profile that, while overlapping with known IBD, has distinct features.

In this study, experiments performed were designed to: (1) confirm the earlier differential gene expression results from inflamed gastrointestinal biopsy tissue in autism spectrum disorder children compared to non-inflamed tissue from typically developing children and, (2) examine the overlap between gene expression in gastrointestinal biopsy tissue and whole blood samples derived from the same patients, in order to identify a putative blood-based test that is diagnostic for ileocolonic inflammation in ASD children.

The study presented herein provides confirmation of the findings of the previous study by replicating this molecular characterization in a second cohort of cases (ASD^{IC+}) and controls (TD^{IC-}). In these two separate case/control mucosal-based cohorts, an overlap of 59 differentially expressed transcripts that are unique to inflamed ileocolonic tissue from GI symptomatic ASD children is demonstrated. Additionally, the study presented herein demonstrates that nine of these 59 transcripts are also differentially expressed in the peripheral blood of ASD^{IC+} children. These nine transcripts therefore represent a blood-base biomarker for ASD-associated ileocolonic inflammation.

The materials and methods used in these experiments are now described.

### Participants

Permission to perform this study was obtained from the Wake Forest University Health Sciences Institutional Review Board (IRB approvals: #IRB00007834 (control samples) and #BG03-464 (ASD samples)). Forty-eight sample sets from this IRB-approved study tissue bank were selected based on presence/absence of inflammation in the relevant tissue sample. For each subject, either 1 or 2 biopsies, and a single sample consisting of 2.5 ml whole blood (collected into a PaxGene Blood RNA tube (PreAnalytiX)), were processed.

### Study Design

Case (ASD^{IC+}; N=21) & Control (TD^{IC-}; N=24)
1. ASDTI
   TI-inflamed (9)
   ASDTI blood (12 {same individuals})
2. ASDcolon
   colon-inflamed (9)
   ASDcolon blood (12 {same individuals})
3. ASDTI & ASDcolon
   TI & colon-inflamed (3)
   blood (3 {same individuals})
4. TDTI
   TDTI-not inflamed-‘control’ (12)
   TDTI blood (12 {same individuals})
5. TDcolon
   TDcolon-not inflamed-‘control’ (12)
   TD colon blood (12 {same individuals})

### Microarray Assay

Total RNA was isolated from each biopsy tissue sample as described earlier (Walker et al, 2013, PLOS One 8:e58058). Briefly, total RNA was isolated from mucosal biopsies that had been stored in RNAlater by sonicating the tissue in the presence of TriReagent® reagent (Molecular Research Center, Inc., Cincinnati, Ohio) according to the method of Chomcynski and Sacchi (Chomcynski et al., 1987, Anal Biochem 162(1): 156-159). Total RNA was purified using RNeasy® Minelute Plus columns (includes an on-column DNAse step) and reagents (Qiagen, Valencia, CA) and eluted in nuclease-free water. RNA concentration and quality were determined using a Nanodrop™ ND-1000 spectrophotometer (Nanodrop Technologies, Wilmington, DE) and Agilent Bioanalyzer, respectively. A single biopsy specimen was typically 3-5 mg of tissue and yielded from 3-10 μg of high quality (e.g. RIN≥7) total RNA.

For the PaxGene blood tubes, total RNA was isolated in a QIAcube robotic workstation using RNeasy® Plus kits (Qiagen, Valencia, CA) following the manufacturer's protocols. RNA quantity and relative quality was assessed using a Nanodrop™ ND-1000 spectrophotometer. RNA integrity was determined using a bioanalyzer (Agilent Technologies, Palo Alto, CA). Total RNA for each sample (0.5-2.0 μg; RIN≥7) was delivered to the Center for Genomics and Personalized Medicine Research Core Facility (Wake Forest Baptist Medical Center) for microarray assay, where labeled cDNA, generated from total RNA, was assayed on Illumina HT v4 BeadArray microarrays (Illumina Inc.). Following hybridization, washing, and scanning, data were extracted from scanned images using Genome Studio Software (Gene Expression module; Illumina Inc.) and processed for upload to gene expression analysis software.

### Statistical Analysis

Following normalization (quantile) and log 2 transformation of the raw data, unsupervised hierarchical clustering and analysis of variance (ANOVA) were performed, using Qlucore Omics Explorer software (Qlucore, Lund, Sweden), to generate principal component analysis (PCA) plots and heat maps. Individual pair-wise comparisons were performed using Student's t-test (fold change≥1.5; p≤0.05) and groups of differentially-expressed genes (DEGs) were compared between groups using GeneSifter® Analysis Edition software (Perkin Elmer). A receiver operating characteristic (ROC) curve analysis was also performed to evaluate the predictive performance of the most discriminating biomarkers in the peripheral blood.

### Pair-Wise Analysis to Determine Differentially-Expressed Transcripts

Gene expression data were uploaded into the GeneSifter® Analysis Edition software (Geospiza, Inc, Seattle, WA) software suite. For pair-wise comparisons the data were log transformed (log base 2) prior to performing the Student's t-test, to generate lists of differentially-expressed transcripts (DETs). The fold-change threshold was set at 1.5 and the data were considered significant if the comparison had an associated log ratio p-value≤0.05.

### Principal Component Analysis

In order to determine the overall similarity between samples, ratio data were subjected to Principal Components Analysis (PCA) and two-way agglomerative cluster analysis using Ward's minimum variance as heuristic criteria and Pearson correlation as the distance metric for experiments, and average linkage as heuristic and Pearson correlation distance as the distance metric for genes to determine the overall similarity between samples and within groups. No filtering was applied to the profile level data prior to PCA. These analyses were performed using Qlucore Omics Explorer software (Lund, Sweden). This study examined gene expression in histologically inflamed colonic and/or ileal intestinal mucosal tissue, as well as whole blood, from GI symptomatic children undergoing diagnostic ileocolonoscopy and biopsy for active GI symptoms (Table 1). Subjects included children with a diagnosis of ASD (ASD^{IC+}; n=21; mean age=8.11 years old ±4.86; 17 males and 4 females). The control group consisted of typically-developing children who underwent diagnostic ileocolonoscopy for chronic GI symptoms in which no histopathology was found (n=24; mean age=12.66 years old±4.08; 18 males and 6 females).

### Case Selection and Biopsy Procurement

The ASD^{IC+} group was selected based upon a history of normal development for at least 12 months followed by developmental regression and onset of GI symptoms. For all individuals in this group, this was their first diagnostic ileocolonoscopy and no patients were taking medication thought to alter the histologic appearance of the GI mucosa. All cases had histologically-confirmed ileitis, colitis, or both in at least one of seven collected and archived colonic biopsies. All patients were assigned a diagnosis of either autism (N=13), ASD (N=4) or PDD-NOS (N=4) (Table 1), given by one or more practitioners from the following specialties: pediatric neurology, developmental pediatrics, pediatric psychiatry or psychology. A detailed history of GI symptoms was documented (Table 2). Patients who met clinical criteria for diagnostic ileocolonoscopy and biopsy and whose parents agreed to participate in this IRB-approved study (Copernicus Group Independent Review Board; WFU1-11-081) were provided with a study description and provided fully informed, written consent. Informed written consent from the next of kin, care givers or guardians on the behalf of all the minors/children participants involved in all studies was obtained. Specimens were obtained using a standard disposable forceps biopsy device, in accordance with routine diagnostic biopsy protocol. Immediately upon procurement of biopsy tissue, a specimen from each of seven anatomic locations (from the terminal ileum to rectum) was processed for paraffin embedding and subsequent routine histopathology. Biopsies for microarray analysis were obtained from the divided mucosal specimen at each anatomic location. These tissues were placed directly into RNAlater® solution (Qiagen Inc; Valencia, CA) and stored at −80° C. prior to processing.

TABLE 1
Clinical and demographic characteristics of the study population

| | Cases | Controls |
|---|---|---|
| | ASD with | TD without |
| | inflammation | inflammation |
| Number | 21 | 24 |
| Age (years) | | |
| Mean (SD) | 8.11 (4.86) | 12.66 (4.08) |
| Range | 2.3-19.25 | 2.08-17.9 |
| Gender | | |
| Male (%) | 17 (81) | 18 (75) |
| Female (%) | 4 (19) | 6 (25) |
| Diagnosis | | |
| Autism | 13 | — |
| ASD | 4 | — |
| PDD-NOS | 4 | — |

TABLE 2
Gastrointestinal symptoms in the ASP study population

| | ASD^{IC+} N = 21 |
|---|---|
| | n (%) |
| Abdominal pain | 18 (86) |
| Diarrhea | 16 (76) |
| Constipation | 8 (38) |
| Food sensitivities | 8 (38) |
| Abdominal distention | 7 (33) |
| Failure to thrive | 5 (24) |

### Control Biopsy Procurement

Prospective controls (Table 1) were recruited through an IRB approved protocol (Wake Forest University Health Sciences Institutional Review Board; #IRB00007834) from the Pediatric Gastroenterology Clinic at the Wake Forest University Health Sciences. Non-IBD Control subjects were further defined as those who, following ileocolonoscopy, were without endoscopic or pathologic findings explaining their symptoms. However, the initial indication for ileocolonoscopy was presence of unexplained GI symptoms ranging from abdominal pain, diarrhea, malnutrition, blood observed in the stools, etc. Failure to diagnose the etiology of observed symptoms by endoscopy was subsequently followed by clinical reassessment or additional diagnostic testing.

No concerns regarding developmental delays for any participant in the control group were reported by parents, relatives, caretakers, or teachers and none were noted by physicians at the Wake Forest Pediatric GI Clinic.

Tissues for microarray analysis were collected, processed and stored in identical fashion to those from children with ASD. Informed written consent from the next of kin, care givers or guardians on the behalf of all the minors/children participants involved in all studies was obtained.

All specimens (cases and controls) have been collected and stored in identical fashion (e.g. pinch cold biopsy forceps, immediate placement in RNA later, and long-term storage at −20 degrees within 24-48 hours post-collection). Cases were collected at a two locations (Far Rockaway, New York and Austin, Texas) with controls collected at a third location (Winston Salem, NC), and both cases and controls were collected using identical specimen collection protocols as outlined in the SOP submissions to the respective IRB's.

The results of experiments are now described.

### Demographics of Cases and Controls

### ASD^{IC+} Samples

Samples from twenty one children with a diagnosis of ASD (Table 1), chronic unexplained GI symptoms (Table 2) and histologic inflammation of either the terminal ileum (N=9), the colon (N=9), or both locations (N=3) were selected for this study. For each individual, ileal and/or colonic biopsies and a sample of whole blood were collected and processed.

### Non-ASD (TD^{IC-}) Controls

Samples from twenty four GI symptomatic non-ASD typically developing children without identifiable histologic inflammation on any biopsies in either the ileum or colon (TD^{IC-}) were included in this study (Table 1). For each control individual, a terminal ileum or a colonic biopsy specimen, as well as a whole blood sample, was collected and processed.

### Principal Component Analysis (PCA)

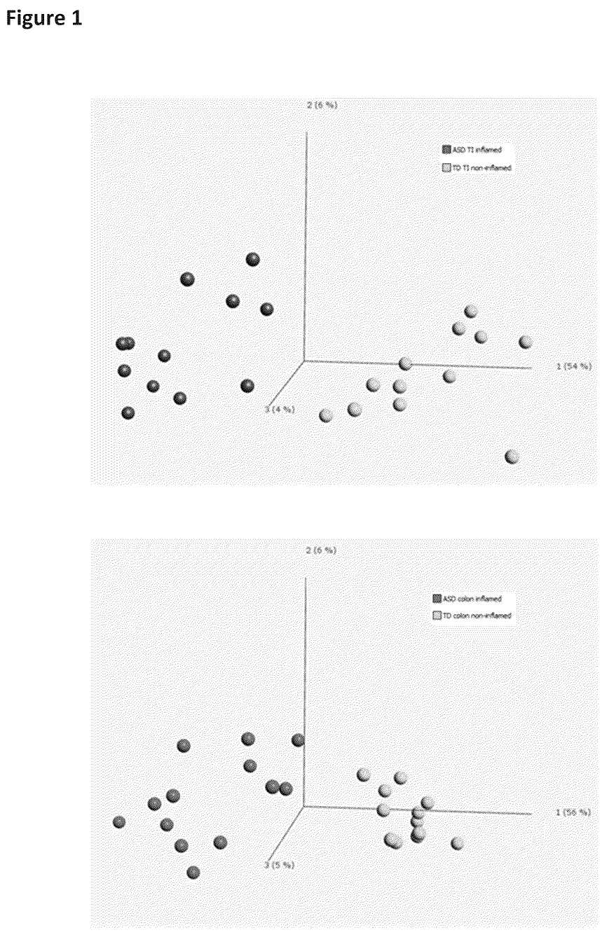

Unsupervised PCA and hierarchical clustering of the sample level data were performed to determine variability/similarity among samples. No filtering was applied to the profile level data prior to PCA. In this current GI mucosal-based replication study gene expression profiles of inflamed ASD GI mucosal tissue (ASD^{IC+}) were compared to non-inflamed TD mucosal tissue (TD^{IC-}). There were 21 samples in the ASD^{IC+} (1 sample from 9 children with histologic ileitis, 1 sample from 9 children with histologic colitis, and 2 samples (1 from the ileum, 1 from the colon) each from 3 children with ileocolitis) and 24 samples (12 from terminal ileum; 12 from colon) in the non-ASD, non-inflamed group. As in the original study (Walker et al., 2013, PLOS One 8:e58058), a clear segregation of GI mucosal derived gene expression profiles emerged between the inflamed and non-inflamed biopsy samples is apparent (FIG. 1).

### Ileal Mucosal Gene Expression Profiles

In the PCA plot illustrating the findings in ileal mucosa, profiles from the 12 TD control children without identifiable ileal (nor colonic) inflammation cluster together in one area of the plot while gene expression profiles from inflamed ileal mucosa representing the ASD^{IC+} samples also cluster together, but apart from controls (FIG. 1—top panel).

### Colonic Mucosa Gene Expression Profiles

In the PCA plot comparing gene expression profiles in colonic mucosa, samples from the 12 TD control children without identifiable colonic (nor ileal) inflammation cluster relatively tightly together, while inflamed colonic mucosa representing the ASD^{IC+} samples show a separate and much broader distribution (FIG. 1—bottom panel).

### Overlapping Gene Expression Profiles from Two Mucosal Based Studies

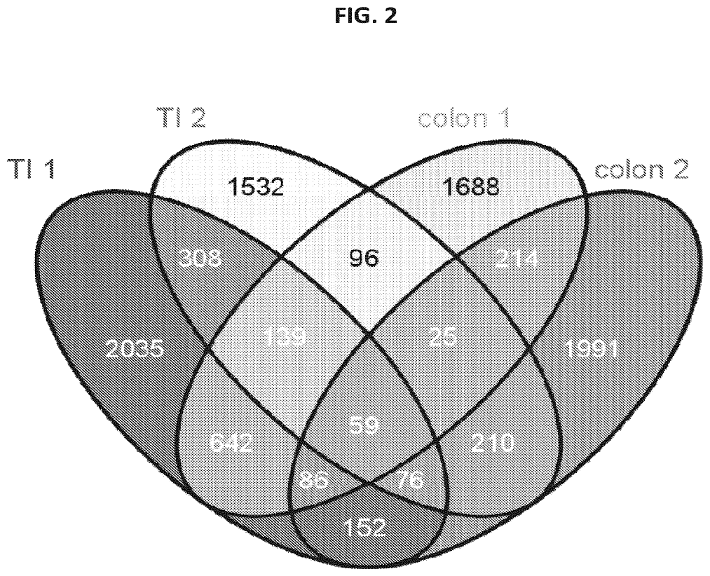

When differential gene expression in GI ileocolonic mucosa biopsy tissue between ASD^{IC+} and non-ASD^{IC-} from our earlier published study (Walker et al., 2013, PLOS One 8:e58058) were compared with the results from this confirmation study, 59 transcripts that were consistently differentially expressed only in inflamed tissue from ASD patients, but not in anatomically matched TD^{IC-} controls were found (FIG. 2; Table 4).

TABLE 4
Transcripts that are differentially expressed in inflamed
ileum and colon in ASD^{IC+} cases from two studies

| Gene Name | Gene Identifier |
|---|---|
| neurotensin | NM_006183 |
| V-set and immunoglobulin domain containing 2 | NM_014312 |
| neuropeptide Y | NM_000905 |
| gamma-aminobutyric acid type A receptor | NM_000807 |
| alpha2 subunit | |
| aquaporin 11 | NM_173039 |
| testis-specific transcript, Y-linked 15 | NR_001545 |
| (non-protein coding) | |
| WAP four-disulfide core domain 1 | NM_021197 |
| ephrin-A1 | NM_004428 |
| interleukin 2 receptor subunit alpha | NM_000417 |
| Fc fragment of IgE receptor Ia | NM_002001 |
| solute carrier family 22 member 4 | NM_003059 |
| cytochrome P450 family 2 subfamily S | NM_030622 |
| member 1 | |
| meprin A subunit alpha | NM_005588 |
| N-acetylated alpha-linked acidic dipeptidase- | NM_005468 |
| like 1 | |
| RAB17, member RAS oncogene family | NM_022449 |
| biotinidase | NM_000060 |
| solute carrier family 26 (anion exchanger), | NM_000112 |
| member 2 | |
| vasoactive intestinal peptide receptor 1 | NM_004624 |
| transmembrane channel like 4 | NM 144686 |
| meprin A subunit beta | NM_005925 |
| glucosidase, beta, acid 3 (gene/pseudogene) | NM_020973 |
| period circadian clock 3 | NM_016831 |
| troponin C2, fast skeletal type | NM_003279 |
| ornithine carbamoyltransferase | NM_000531 |
| glutathione S-transferase mu 2 (muscle) | NR_002932 |
| pseudogene 1 | |
| actin binding LIM protein family member 2 | NM_032432 |
| thyrotrophic embryonic factor | NM_003216 |
| formyl peptide receptor 1 | NM_002029 |
| epoxide hydrolase 1 | NM_000120 |
| tumor necrosis factor receptor superfamily | NM_016639 |
| member 12A | |
| interleukin 1 receptor antagonist | NM_173842 |
| ribosomal protein L39 like | NM_052969 |
| LDL receptor related protein 8 | NM_033300 |
| cyclic nucleotide gated channel alpha 1 | NM_000087 |
| Cdk5 and Abl enzyme substrate 1 | NM_138375 |
| membrane spanning 4-domains A10 | NM_206893 |
| high mobility group box 2 | NM_002129 |
| centromere protein L | NM_033319 |
| dishevelled-binding antagonist of beta-catenin 2 | NM_214462 |
| TNF alpha induced protein 3 | NM_006290 |
| zinc finger protein 358 | NM_018083 |
| polo like kinase 4 | NM_014264 |
| shugoshin-like 2 (S. pombe) | NM_152524 |
| TIMELESS interacting protein | NM_017858 |
| kinesin family member 14 | NM_014875 |
| solute carrier family 27 member 2 | NM_003645 |
| centromere protein E | NM_001813 |
| methylenetetrahydrofolate dehydrogenase 2, | NM_006636 |
| methenyltetrahydrofolate cyclohydrolase | |
| ubiquitin like with PHD and ring finger | NM_013282 |
| domains 1 | |
| SPC25, NDC80 kinetochore complex | NM_020675 |
| component | |
| sulfiredoxin 1 | NM_080725 |
| sialic acid binding Ig like lectin 17, | NR 002804 |
| pseudogene | |
| replication factor C subunit 3 | NM_002915 |
| BUB1 mitotic checkpoint serine/threonine | NM_001211 |
| kinase B | |
| helicase, lymphoid-specific | NM_018063 |
| non-SMC condensin I complex subunit G | NM_022346 |
| cytochrome P450 family 51 subfamily A | NM_000786 |
| member 1 | |
| calcium voltage-gated channel auxiliary subunit | NM_172364 |
| alpha2delta 4 | |
| paired like homeodomain 1 | NM_002653 |

### Blood Gene Expression Profiles

Peripheral blood gene expression is being used extensively to identify surrogate markers for disease and, relevant to this study, specifically for discrimination and classification of inflammatory bowel disease. This approach is based on the premise that peripheral blood, as it interacts with the tissues and cells of the body, may manifest gene expression changes in response to or because of the diseased tissue they encounter. The second part of the current study was designed to identify transcripts that are differentially expressed in peripheral blood of ASD^{IC+} patients compared to peripheral blood obtained from TD^{IC-} controls. Both ASD^{IC+} and TDIC-control blood was obtained from the same patients, and at the same time, as their respective mucosal tissue. The resulting ASD^{IC+}-specific peripheral blood gene expression profile was also compared to DETs that were uniquely expressed in the inflamed ileocolonic mucosal tissue from those same individuals as well as from the original case/control cohort (59 overlapping mucosal DETs; FIG. 2).

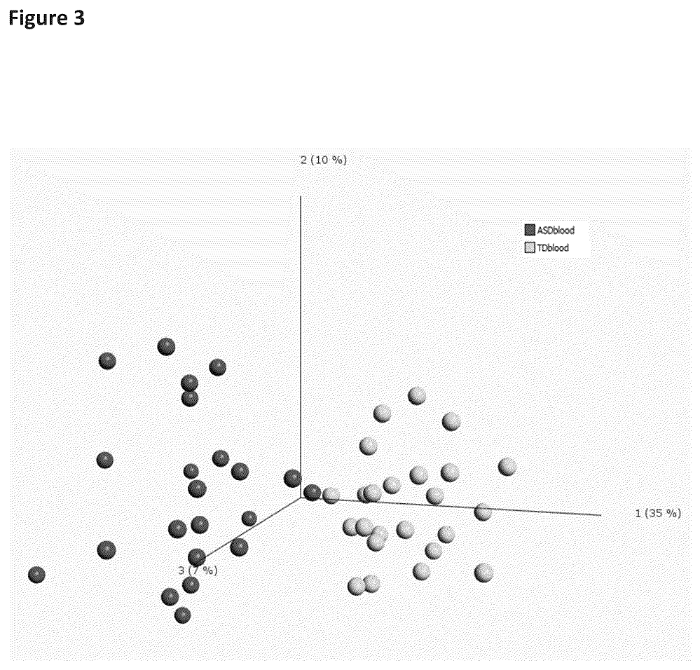

Similar to what we found with gene expression in the ilecolonic mucosal tissue sample comparisons, the PCA plots reveal a clear separation between cases and controls in the peripheral blood gene expression profiles (FIG. 3).

### Overlapping Differential Gene Expression in Blood and GI Biopsy Tissue in ASD^{IC+} Samples

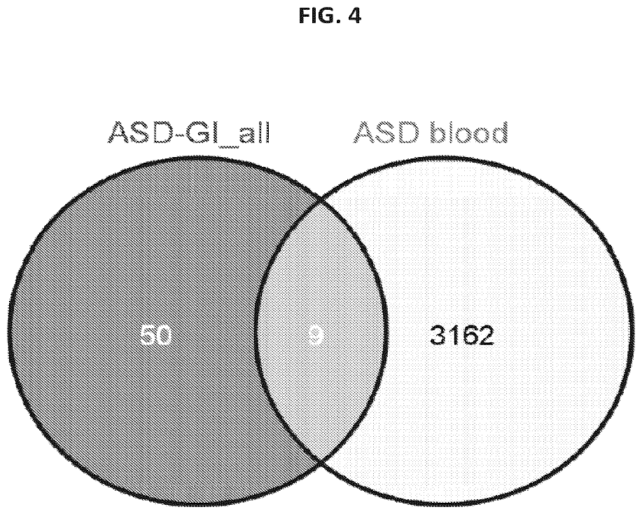

A pairwise comparison of blood gene expression in ASD^{IC+} samples compared to controls revealed 3171 transcripts that were uniquely differentially-expressed in the blood of ASD^{IC+} individuals. When the overlap of uniquely differentially expressed transcripts (DETs) in the inflamed ileocolonic mucosal tissue from both of the mucosal-based studies were examined, 59 DETs were identified (FIG. 2). Further comparison between these 59 mucosal-based DETs (both studies) with the blood-based DETs (from this study only) revealed nine transcripts that were differentially expressed in both blood and inflamed mucosa in all of the ASD^{IC+} cases (FIG. 4; Table 3). Four of these nine DETs are up-regulated in both ileum and colon in all samples from both studies, and four are down-regulated. One DET, TNFRSF12A, is up-regulated in colon, but down-regulated in terminal ileum (Table 3). In the peripheral blood, the direction of differential expression (up- or down-regulated) of these nine transcripts matches that in the mucosal tissue in five of nine instances (Table 3). PCR validation of representative DETs in these studies has shown significant agreement with microarray-based findings (Walker et al., 2013, PLOS One 8:e58058).

TABLE 3
Differential expression (fold change in cases versus controls)
of nine transcripts in tissues from two independent studies.

| | | Study #1 | | Study #2 | | |
|---|---|---|---|---|---|---|
| Accession | Gene | ASD- | ASD- | ASD- | ASD- | |
| No. | Symbol | TI | colon | TI | colon | blood |
| NM_002001 | FCER1A | 2.69↓ | 3.52↓ | 3.11↓ | 1.94↓ | 1.55↓ |
| NM_030622 | CYP2S1 | 2.56↓ | 2.76↓ | 2.31↓ | 1.63↓ | 2.47↓ |
| NM_144686 | TMC4 | 2.36↓ | 1.59↓ | 2.11↓ | 1.55↓ | 3.16↑ |
| NM_016639 | TNFRSF12A | 1.96↓ | 3.16↑ | 1.75↓ | 2.98↑ | 2.95↓ |
| NM_173842 | IL1RN | 1.94↑ | 3.03↑ | 6.91↑ | 4.22↑ | 1.41↓ |
| NM_006290 | TNFAIP3 | 1.75↑ | 1.62↑ | 1.67↑ | 1.53↑ | 1.26↑ |
| NM_001813 | CENPE | 1.66↑ | 2.71↑ | 1.77↑ | 1.51↑ | 3.46↓ |
| NM_006636 | MTHFD2 | 1.66↑ | 2.25↑ | 1.85↑ | 1.83↑ | 1.78↓ |
| NR_002804 | SIGLEC17P | 1.63↓ | 1.81↓ | 3.69↓ | 4.83↓ | 3.64↓ |

### Receiver Operating Characteristic (ROC) Curve Analysis

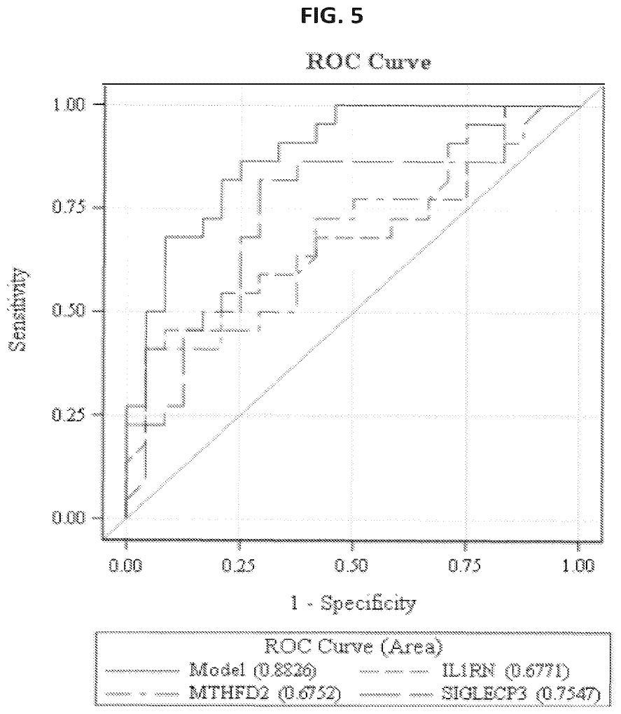

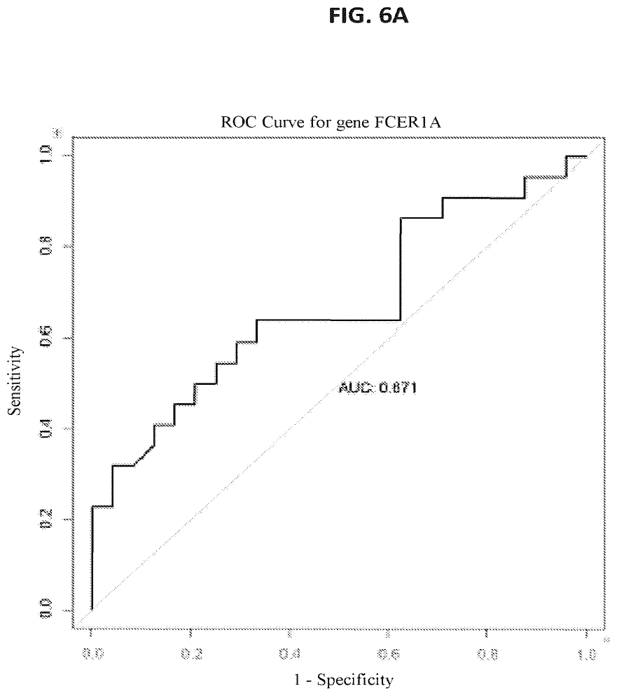

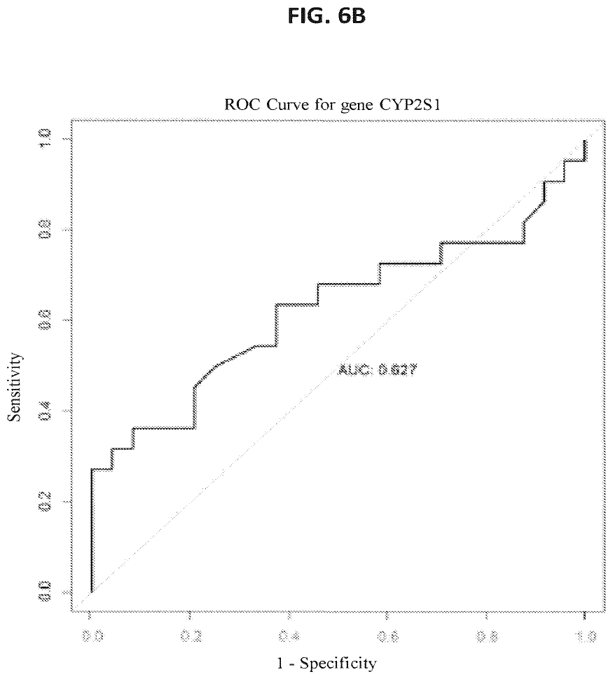

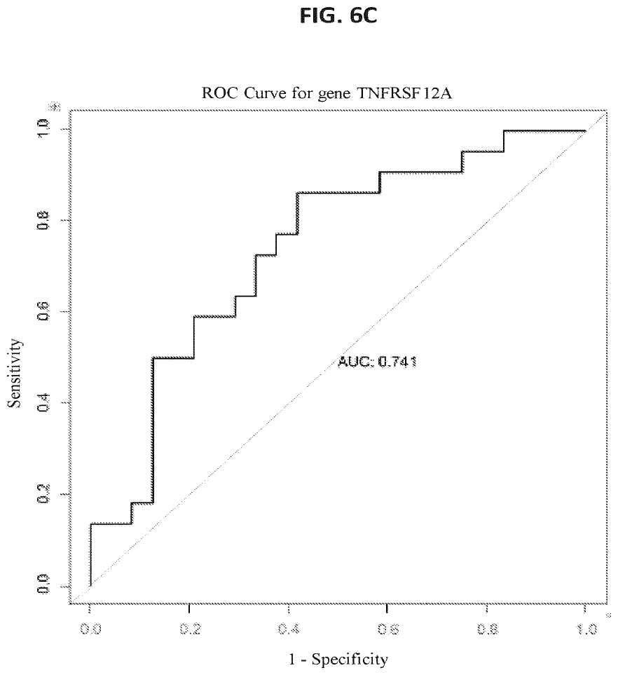

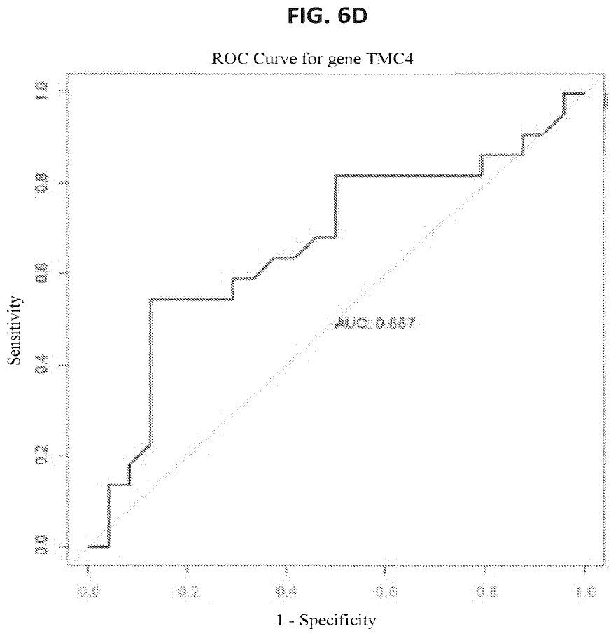

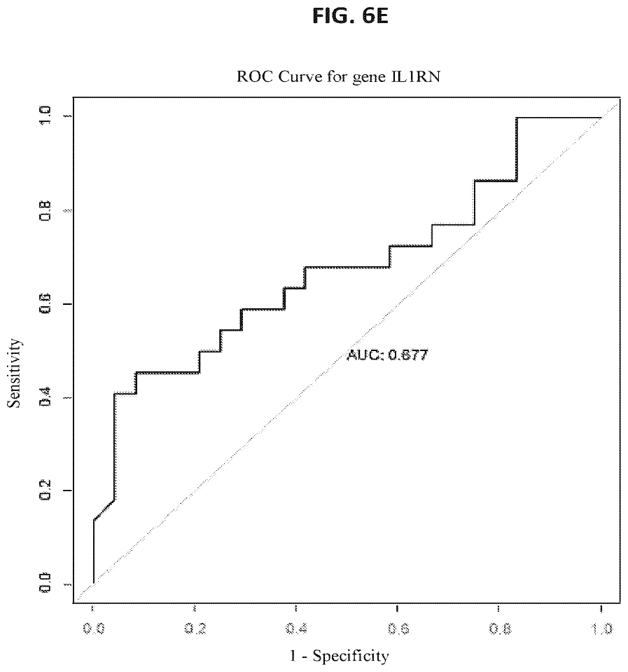

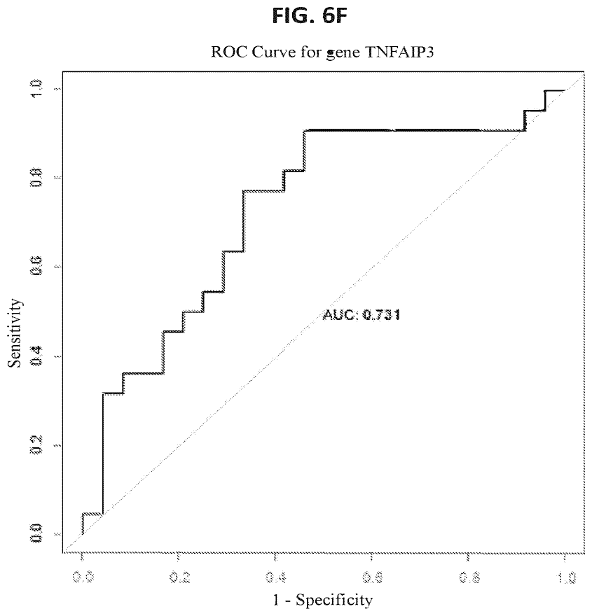

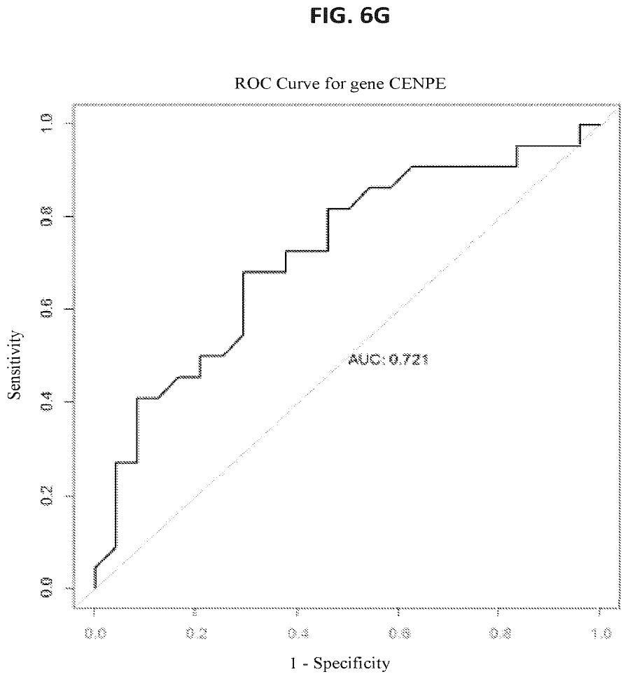

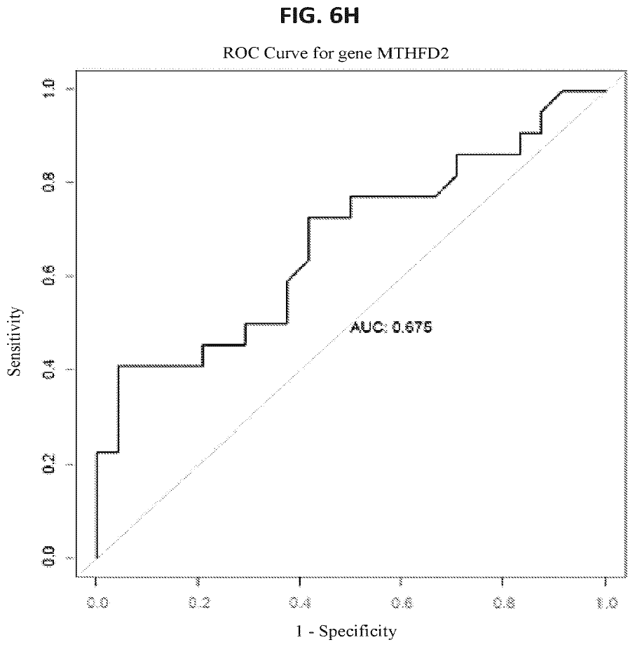

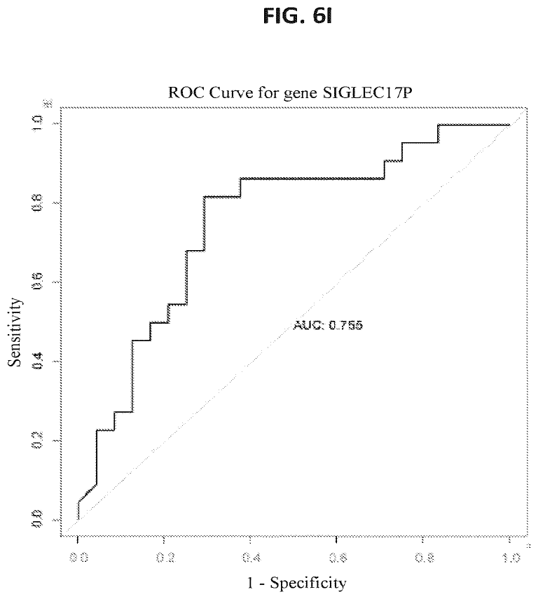

An exploratory ROC analysis of the differentially expressed blood and GI tissue transcripts both univariately and in multivariable linear combinations was performed to evaluate the predictive capability of the blood-based biomarkers. Univariate ROC curve AUC values ranged from 0.627 to 0.755 among the 9 differentially expressed blood-based transcripts (FIGS. 6A-6I). A candidate composite biomarker was created using stepwise variable selection to identify a linear combination of transcripts that maximizes the AUC of a ROC curve, including biomarkers that are significant at a 0.05 level. The variable selection procedure identified a linear combination of three transcripts that together yielded an AUC of 0.883 (FIG. 5).

### Blood-Based Biomarker for ASD-Associated Ileocolitis in GI Symptomatic ASD Children

Described in the study herein is a blood-based biomarker set (derived from 2 cohorts: cohort #1 (Walker et al., 2013, PLOS One 8:e58058) and cohort #2 (this study)) containing nine mRNA transcripts that reflects the presence of ASD-associated ileocolitis in GI symptomatic ASD children. The findings from cohort #2 demonstrate significant overlap with, and provide validation of, the results from our initial cohort in which we described bowel tissue derived differentially expressed transcripts uniquely present in inflamed ileocolonic tissue from GI symptomatic ASD children (i.e. not found in non-ASD pediatric Crohn's disease, non-ASD pediatric ulcerative colitis, nor in typically-developing children without evidence of intestinal inflammation). Differential expression of fifty nine transcripts was consistently seen in the ASD^{IC+} in comparisons between GI mucosal tissue (ileum and colon) from the two distinct ASD^{IC+} cohorts and two distinct TDIC-control groups. A subset consisting of nine DETs of these fifty nine mucosal-based transcripts was also found to be differentially expressed in blood (from the cohort #2 only—not measured in the initial study). Moreover, the majority of the nine transcripts identified in both ileocolonic biopsy tissue and blood from ASD^{IC+} children encode proteins that are associated with biologic processes known to be impacted in children with autism, suggesting the peripheral blood marker may not only provide a proxy for GI inflammation, but may provide functional insights as well.

A study was designed herein that capitalizes on the strengths of each of these approaches and have evaluated both the affected organ tissue and blood, taken at the same time and from the same (living) individuals, to identify a clinically-relevant disease biomarker, in a readily-accessible peripheral tissue, for an especially vulnerable patient population. In the studies herein, a subgroup of ASD children with histopathologically-confirmed gastrointestinal inflammation and, using gene expression profiles in tissue from the inflamed gastrointestinal mucosa compared to expression profiles in peripheral blood from the same cases and controls, identified a putative gene expression profile that can be used by a pediatric gastroenterologist to assess the probability of ASD^{IC+} and as an aid in determining which patients are in need of a more comprehensive work up.

As a follow-up to an earlier identification of unique ASD ileocolitis-associated mucosal tissue based gene expression (Walker et al., 2013, PLOS One 8:e58058), the studies described herein sought to determine which of the (178) DETs from the original cohort were also differentially-expressed in mucosal tissue and peripheral blood within a second cohort. The strategy for identifying a clinically-relevant peripheral biomarker for ASD^{IC+} was based upon the premise that, whenever possible, biomarker discovery should begin in tissue that demonstrates known (and unique) disease-associated pathology (in this case—inflamed GI mucosal tissue) to first identify a disease-specific signature (Walker et al., 2013, PLOS One 8:e58058). This is then followed by analysis of peripheral blood, a tissue which has not been shown to be a “diseased tissue” in autism or ASD^{IC+}, but rather functions as a more readily accessible proxy tissue. Overlap of differential expression of specific transcripts within the disease tissue and the proxy tissue provides additional confidence that the peripheral biomarker has validity and clinical relevance (Glatt et al., 2005, Proc Natl Acad Sci USA 102(43):15533-15538).

Although there are dozens of publications reporting the identification of blood-based biomarkers for diseases such as obstructive coronary artery disease (Voros et al., 2014, Atherosclerosis 233(1):284-290), Huntington's disease (Mastrokolias et al., 2015, Eur J Hum Genet 23(10):1349-1356; Lovrecic et al., 2009, Mov Disord 24(15):2277-2281; Borovecki et al., 2005, Proc Natl Acad Sci USA 102(31):11023-11028), multiple sclerosis (Gandhi et al., 2010, Hum Mol Genet 19(11):2134-2143), epilepsy and new-onset idiopathic pediatric epilepsy (Karsten et al., 2011, Neurosci Lett 497(3):213-217; Greiner et al., 2013, Epilepsia 54(2):272-279), and recent-onset juvenile idiopathic arthritis (Barnes et al., 2009, Arthritis Rheum 60(7):2102-2112), to name a few, there are only limited numbers of studies that have used gene expression in the target “disease” tissue, correlated with peripheral blood gene expression in the same individuals, obtained at the same time, as a strategy to identify a blood-based biomarker. It is believed this study represents the first effort to use such an approach in ASD children with gastrointestinal inflammation.

The examination of both terminal ileum and colonic specimens in obtaining the initial data set was of great importance as the ASD-associated ilcocolitis has been observed to be present in both the small bowel and colon. Evaluation of gene expression in both anatomic locations allowed identification of DETs that are common to both the terminal ileum and large intestine and helped to create an initial data set that did not include transcripts whose differential expression may not reflect disease but rather the site of origin of the tissue (i.e. seen only in TI or seen only in colon).

The majority of the transcripts that comprise the putative blood-based biomarker have functions that are known to be relevant in either ASD, inflammation, or both. For example IL1RN (interleukin 1 receptor antagonist), a potent anti-inflammatory molecule that inhibits the activities of IL1α and IL1β and modulates a variety of interleukin-1 related immune and inflammatory responses, is upregulated in inflamed GI tissues and down-regulated in the peripheral blood. Elevated IL1RN in the inflamed gastrointestinal mucosa makes biological sense in the context of the body's attempt to modulate the damaging effects of the proinflammatory interleukin-1 in the gut and the well-established role of IL-1 in the pathogenesis of GI-related inflammatory disorders (Lopetuso et al., 2013, Front Immunol 9(4):1-20). Lower circulating levels of IL1RN may constitute a peripheral signal of the active inflammatory response occurring in the GI tract.

In addition to its role in GI inflammation, IL-1 is also known to play a major role in neuroinflammation (Moynagh et al., 2005, J Anat 207(3):265-269) and has been shown to contribute to neuroinflammatory-associated breakdown of the blood brain barrier (Hofman et al., 1986, J Immunol 136(9):3239-3245). The IL-1 family of cytokines is one of many proinflammatory cytokines present in excess in children with autism (Krakowiak et al., 2015, Biol Psychiatry 2015 Aug. 14. pii: S0006-3223(15)00655-1; Heuer et al., 2008, Autism Res 1(5):275-283; Ashwood et al., 2011, Brain Behav Immun 25(1):40-45) and both neuroinflammation and deficits in blood brain barrier function have been implicated in the pathogenesis of ASD related brain dysfunction (Theoharides et al., 2011, J Neuroinflammation 6(166):1-5).

An important cytokine receptor transcript in this putative biomarker, TNFRSF12A (tumor necrosis factor receptor superfamily 12A), is over-expressed in inflamed colonic tissue and down-regulated in both the terminal ileum and peripheral blood of ASD^{IC+} cases. This receptor (aka Fn14) binds the tumor necrosis factor superfamily member TWEAK (TNF-like weak inducer of apoptosis), a pro-inflammatory cytokine implicated in tissue regeneration and wound repair (Brown et al., 2013, PLOS One 8(6):1-11). The binding of TWEAK to its receptor activates several signaling cascades including the NF-κB pathway, and sustained Fn14 signaling has been implicated in the pathogenesis of chronic IBD. Moreover, it has been postulated that TWEAK-independent Fn14 signaling may occur in instances where Fn14 levels are highly elevated (Brown et al., 2013, PLOS One 8(6):1-11). Interestingly, elevated Fn14 expression correlates highly with elevated MET (hepatocyte growth factor receptor that encodes tyrosine kinase activity) in a form of metastatic cancer, and depletion of Fn14 is sufficient to inhibit MET driven tumor cell migration and invasion in vitro (Whitsett et al., 2014, Clin Exp Metastasis 31(6):613-623). The human MET gene is a well-established risk factor for ASD that functions in both brain development and gastrointestinal repair, and has been shown to confer a distinct risk in families with co-occurring autism and gastrointestinal conditions (Peng et al., 2016, Mol Psychiatry 182:1-11; Campbell et al., 2009, Pediatrics 123(3):1018-1024).

Another key signaling molecule, TNFAIP3 (tumor necrosis factor, alpha-induced protein 3), a transcript also up-regulated both in GI tissue and blood, is rapidly induced by TNF and has been shown to inhibit NF-κB activation as well as TNF-mediated apoptosis. TNFα has been shown to be present in both peripheral lymphocytes and inflamed ASD small and large intestinal mucosal tissue in excess of that found in TD^{IC-} and TD Crohn's disease (Ashwood et al., 2004, J Clin Immunol 24:664-673; Ashwood et al., 2006, J Neuroimmunol 173:126-134; Jyonouchi et al., 2001, J Neuroimmunol 120(1-2):170-179). Moreover, variants of TNFAIP3 are also known risk factors for celiac disease and are implicated in altered NF-κB signaling (Trynka et al., 2009, Gut 58(8):1078-1083).

The high affinity IgE receptor FcεR1 (consisting of one α subunit, one β subunit, and two γ subunits) is constitutively expressed in mast cells and basophils and initiates the allergic response upon interaction with allergens (Greer et al., 2014, J Clin Invest 124(3):1187-1198). In humans, but not rodents, FcεR1 is also constitutively expressed in dendritic cells and monocytes, although this form of receptor is trimeric (lacking the β subunit) and it has been proposed that on dendritic cells, the receptor promotes immune homeostasis and regulation (Shin et al., 2015, Cell Mol Life Sci 72(12):2349-2360). The studies herein found that the gene encoding the alpha subunit for this receptor, FCER1A, was down-regulated in both the mucosal tissue and peripheral blood from ASD^{IC+} cases. Without intending to be bound by theory, it is believed this finding further supports the concept of an imbalance in immune homeostasis in ASD^{IC+}.

A key mitochondrial folate pathway gene, MTHFD2 (methylenetetrahydrofolate dehydrogenase (NADP+dependent) 2, methenyltetrahydrofolate cyclohydrolase) encodes a nuclear-encoded bifunctional mitochondrial enzyme and was found herein to be up-regulated in inflamed GI tissue and down-regulated in peripheral blood in ASD^{IC+} cases. Importantly, while this gene is expressed in 13 developing embryos, it is typically absent in most healthy adult tissues. MTHFD2 RNA and protein have been shown, however, to be markedly elevated in many cancers and negatively correlated with survival in breast cancer (Nilsson et al., 2014, Nat Commun 5(3128):1-10). Moreover, mitochondrial dysfunction has been shown to be associated with ASD, although the precise role has yet not been entirely explained (Rossignol et al., 2014, Front Physiol. 22(5):1-15; Frye et al., 2015, Microb Ecol Health Dis 26(27458):1-17).

Finally, CYP2S1 (cytochrome P450, family 2), which encodes an extra-hepatic xenobiotic metabolizing enzyme expressed in high levels in epithelial tissues such as those found in the lung, skin and colon (Deb et al., 2009, Expert Opin Drug Metab Toxicol 5(4):367-380), is down-regulated in blood and in GI tissue of ASD^{IC+} cases. CYP2S1 functions to catalyze reactions involved in drug metabolism and synthesis of cholesterol, steroids, and other lipids and some studies suggest the enzyme may play an important role in modulating inflammation. Whether it acts as in anti-inflammatory or pro-inflammatory mode depends upon the substrate it encounters (Deb et al., 2009, Expert Opin Drug Metab Toxicol 5(4):367-380). Of interest in the context of IBD, CYP2S1 has been shown to be negatively regulated by corticosteroids, specifically the synthetic glucocorticoid dexamethasone, in human cell lines (Bui et al., 2011, Drug Metab Dispos 39(2):180-190). Glucocorticoids are widely used to treat allergic, inflammatory, and autoimmune conditions so this may provide one explanation for the reduction in CYP2S1 observed in the studies herein.

The clinical utility of a blood-based biomarker that predicts, with high probability, the presence of ASD^{IC+} can hardly be overstated. With the frequency of GI symptoms in ASD acknowledged as being more common than in TD children and, in some studies as high as 70%, and with the prevalence of ASD at 1 in 68 American children, the sheer number of GI symptomatic ASD children of any age will be substantial. An as yet unknown, but potentially high, fraction of these will have the ASD^{IC+} phenotype. Because empiric therapy of IBDs will at best provide only short respite (and can even make symptoms worse), and because of the known association between severity of GI symptoms and extremes of ASD behaviors, identifying those children most likely to be ASD^{IC+} is critically important for any pediatric gastroenterologist faced with an ASD child with chronic GI symptoms in making the decision as to whether diagnostic endoscopy is indicated.

Clinical symptom presentation alone does not allow the clinician to distinguish those ASD children with a chronic inflammatory bowel disease from those who do not have a chronic inflammatory process occurring in the bowel (Krigsman et al., 2010, Autism Insights 1:1-11). The specter of subjecting all such GI symptomatic ASD children to diagnostic endoscopy presents a huge logistical problem in the setting of current clinical practice in terms of time needed to be spent with the parents describing the procedure and explaining the need for such diagnostic testing, the difficulty in performing bowel preps in these often uncooperative and poorly communicative children (and young adults), the ability to manage patients with significant behavioral, cognitive, and emotional deficits in the endoscopy suite, the ability to safely anesthetize them, and the ability to manage them in the postoperative area upon awakening from anesthesia in a manner that insures their safety and the safety of other patients. Contemplation of such large-scale diagnostic procedures can be reduced to manageable numbers by initial screening, using biomarkers, so that available resources of time, personnel, and money can be focused on the patients statistically most likely to be diagnosed (predicted by a simple blood test) with ilcocolitis upon biopsy.

Although this current study includes a more age- and gender-matched sample than the original pilot study (Walker et al., 2013, PLOS One 8:e58058), the numbers of cases and controls are still relatively modest and constitute an important study limitation. So although the finding of overlap in the differentially-expressed transcripts from the inflamed GI tissues between the two studies was both encouraging and expected, as was the reduction in the overall number of DETs unique to the ASD^{IC+} samples when combining the two data sets, these findings need to be validated in a much larger sample. The putative nine-transcript biomarker, reflecting the overlap in peripheral blood gene expression with GI mucosal based gene expression, while statistically compelling, will need to be replicated in additional blood samples as the data in this study, relating to blood gene expression, are derived from a single cohort of 21 cases and 24 controls. ROC analyses suggest that a subset of the nine transcripts comprising the putative marker, consisting of three genes (MTHFD2, IL1RN, and SIGLECP3), provides a reasonable level of sensitivity and specificity with a combined AUC of 0.88.

Differences in diet, medications, age, gender, and nutritional supplements used by many children in the study group may affect gene expression of the GI mucosa. Strict control of these factors in an attempt at uniformity is not practical in autism research, and our finding of consistent gene expression profiles across the wide variation of these variables in two distinct study cohorts effectively minimize their potential effect on the validity of the findings described herein.

There are some additional limitations regarding the controls used in these studies. Although peripheral blood from GI symptomatic ASD children can serve as a proxy tissue for ASD^{IC+} (i.e. ileocolonic inflammation in ASD children), the influence of the ASD phenotype itself on the blood gene expression profile cannot be ruled out. That is, the findings herein do not distinguish whether the nine unique blood-based DETs reflect the autism phenotype alone or the autism-plus-ileocolitis phenotype. If the former, the findings would be of even greater clinical significance. To address this, follow-on studies will include whole blood samples from two additional control groups: (1) ASD children without GI symptoms and (2) TD children without GI symptoms.

### Other Embodiments

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

The disclosures of each and every patent, patent application, and publication cited herein are hereby incorporated herein by reference in their entirety. In particular, the disclosure of United States Patent Publication No. 2015-0361499 A1 is incorporated herein by reference in its entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A method of treating a subject having a gastrointestinal disorder, the method comprising administering a treatment for gastrointestinal disorder to a pre-selected subject, wherein the subject is pre-selected by detecting an alteration in the level of a polypeptide or polynucleotide of at least one biomarker selected from the group consisting of FCER1A, CYP2S1, TMC4, IL1RN, TNFAIP3, CENPE, MTHFD2, and SIGLEC17P relative to a reference in a blood sample obtained from the subject.

2. A method of diagnosing a gastrointestinal disorder in a subject, the method comprising measuring the level of a polypeptide or polynucleotide of at least one biomarker selected from the group consisting of FCER1A, CYP2S1, TMC4, IL1RN, TNFAIP3, CENPE, MTHFD2, and SIGLEC17P in a blood sample from the subject, wherein an alteration in the level of the polypeptide or polynucleotide relative to a reference indicates that the subject has a gastrointestinal disorder.

3. The method of claim 1, wherein the subject is pre-selected by further detecting an alteration in the level of TNFRSF12A polypeptide or polynucleotide.

4. The method of claim 2, further comprising measuring the level of TNFRSF12A polypeptide or polynucleotide.

5. The method of claim 1, wherein the subject is pre-selected by detecting an alteration in the level of a IL1RN polypeptide or polynucleotide, MTHFD2 polypeptide or polynucleotide, and SIGLEC17P polypeptide or polynucleotide.

6. The method of claim 2, wherein the method comprises measuring the level of a IL1RN polypeptide or polynucleotide, MTHFD2 polypeptide or polynucleotide, and SIGLEC17P polypeptide or polynucleotide.

7. The method of claim 1, wherein the alteration comprises an increase in the level of TMC4 polypeptide or polynucleotide, an increase in the level of TNFAIP3 polypeptide or polynucleotide, a decrease in the level of FCER1A polypeptide or polynucleotide, a decrease in the level of CYP2S1 polypeptide or polynucleotide, a decrease in the level of TNFRSF12A polypeptide or polynucleotide, a decrease in the level of IL1RN polypeptide or polynucleotide, a decrease in the level of CENPE polypeptide or polynucleotide, a decrease in the level of MTHFD2 polypeptide or polynucleotide, or a decrease in the level of SIGLEC17P polypeptide or polynucleotide.

8. The method of claim 1, wherein the gastrointestinal disorder is ileocolitis, ileitis, colitis, enteritis, duodenitis, gastritis, and/or esophagitis.

9. The method of claim 1, wherein the subject is a child.

10. The method of claim 1, wherein the subject has an autism spectrum disorder.

11. The method of claim 2, wherein measuring comprises PCR assays or microarrays.

12. The method of claim 2, further comprising a step of using the results obtained from the diagnostic assay to selecting or administering a treatment.

13. The method of claim 1, wherein the treatment comprises corticosteroids, immunomodulators, 5-aminosalicylic acid preparations, cytokine specific antagonists, dietary restrictions, antimicrobials, probiotics, and/or supplemental digestive enzymes.

14. A method of diagnosing autism spectrum disorder (ASD) in a subject, the method comprising measuring the level of a polypeptide or polynucleotide of at least one biomarker selected from the group consisting of FCER1A, CYP2S1, TMC4, IL1RN, TNFAIP3, CENPE, MTHFD2, and SIGLEC17P in a blood sample from the subject, wherein an alteration in the level of the polypeptide or polynucleotide relative to a reference indicates that the subject has autism spectrum disorder.

15. The method of claim 14, further comprising measuring the level of TNFRSF12A polypeptide or polynucleotide.

16. The method of claim 14, wherein the method comprises measuring the level of a polypeptide or polynucleotide of biomarkers IL1RN, MTHFD2, and SIGLEC17P.

17. The method of claim 14, wherein the alteration comprises an increase in the level of TMC4 polypeptide or polynucleotide, an increase in the level of TNFAIP3 polypeptide or polynucleotide, a decrease in the level of FCER1A polypeptide or polynucleotide, a decrease in the level of CYP2S1 polypeptide or polynucleotide, a decrease in the level of TNFRSF12A polypeptide or polynucleotide, a decrease in the level of IL1RN polypeptide or polynucleotide, a decrease in the level of CENPE polypeptide or polynucleotide, a decrease in the level of MTHFD2 polypeptide or polynucleotide, or a decrease in the level of SIGLEC17P polypeptide or polynucleotide.

18. The method of claim 14, wherein the autism spectrum disorder is autism.

19. The method of claim 14, wherein said subject is a child.

20. The method of claim 14, wherein measuring comprises PCR assays or microarrays.

21.-25. (canceled)
